SEQUENCE LISTING (12 sequences)

SEQ ID NO: 1 - DNA, 624 bp, Pyrodictium abyssi
gtgaagtacacaaccctagctatagcgggtattattgcctcggctgccgccctcgccctcctagcaggcttcgccaccacccagagccccctcaacagcttctacgccaccggtacagcacaggcagtaagcgagccaatagacgtagaaagccacctcggcagcataacccccgcagccggcgcacagggcagtgacgacataggttacgcaatagtgtggataaaggaccaggtcaatgatgtaaagctgaaggtgaccctgcgtaacgctgagcagctaaagccctacttcaagtacctacagatacagataacaagcggctatgagacgaacagcacagctctaggcaacttcagcgagaccaaggctgtgataagcctcgacaaccccagcgccgtgatagtactagacaaggaggatatagcagtgctctatccggacaagaccggttacacaaacacttcgatatgggtacccggtgaacctgacaagataattgtctacaacgagacaaagccagtagctatactgaacttcaaggccttctacgaggctaaggagggtatgctattcgacagcctgccagtgatattcaacttccaggtgctacaagtaggctaa

SEQ ID NO: 2 - protein, 207 aa, Pyrodictium abyssi
VKYTTLAIAGIIASAAALALLAGFATTQSPLNSFYATGTAQAVSEPIDVESHLGSITPAAGAQGSDDIGYAIVWIKDQVNDVKLKVTLRNAEQLKPYFKYLQIQITSGYETNSTALGNFSETKAVISLDNPSAVIVLDKEDIAVLYPDKTGYTNTSIWVPGEPDKIIVYNETKPVAILNFKAFYEAKEGMLFDSLPVIFNFQVLQVG

SEQ ID NO: 3 - DNA, 513 bp, Pyrodictium abyssi
gtgaagcctacggctctagccctggctggtatcattgcctcggctgccgacctcgccctgctagcaggcttcgccaccacccagagcccgctcaacagcttctacgccaccggcacagcagccgcaacaagcgagccaatagacgtagagagccacctcagcagcatagcccctgctgctggcgcacagggcagccaggacataggctacttcaacgtgaccgccaaggatcaagtgaacgtgacaaagataaaggtgaccctggctaacgctgagcagctaaagccctacttcaagtacctacagatagtgctaaagagcgaggtagctgacgagatcaaggccgtaataagcatagacaagcctagcgccgtcataatactagacagccaggacttcgacagcaacaacagagcaaagataagcgccactgcctactacgaggctaaggagggcatgctattcgacagcctaccgctaatattcaacatacaggtgctaagcgtcagctaa

SEQ ID NO: 4 - protein, 170 aa, Pyrodictium abyssi
VKPTALALAGIIASAADLALLAGFATTQSPLNSFYATGTAAATSEPIDVESHLSSIAPAAGAQGSQDIGYFNVTAKDQVNVTKIKVTLANAEQLKPYFKYLQIVLKSEVADEIKAVISIDKPSAVIILDSQDFDSNNRAKISATAYYEAKEGMLFDSLPLIFNIQVLSVS

SEQ ID NO: 5 - DNA, 537 bp, Pyrodictium abyssi
atgaggtacacgaccctagctctggccggcatagtggcctcggctgccgccctcgccctgctagcaggcttcgccacgacccagagcccgctaagcagcttctacgccaccggcacagcacaagcagtaagcgagccaatagacgtagagagccacctagacaacaccatagcccctgctgccggtgcacagggctacaaggacatgggctacattaagataactaaccagtcaaaagttaatgtaataaagctgaaggtgactctcgctaacgccgagcagctaaagccctacttcgactacctacagctagtactcacaagcaacgccactggcaccgacatggttaaggctgtgctaagcctcgagaagcctagcgcagtcataatactagacaacgatgactacgatagcactaacaagatacagctaaaggtagaagcctactatgaggctaaggagggcatgctattcgacagcctaccagtaatactgaacttccaggtactgagcgccgcttgcagtcccttgtggtga

SEQ ID NO: 6 - protein, 178 aa, Pyrodictium abyssi
MRYTTLALAGIVASAAALALLAGFATTQSPLSSFYATGTAQAVSEPIDVESHLDNTIAPAAGAQGYKDMGYIKITNQSKVNVIKLKVTLANAEQLKPYFDYLQLVLTSNATGTDMVKAVLSLEKPSAVIILDNDDYDSTNKIQLKVEAYYEAKEGMLFDSLPVILNFQVLSAACSPLW

SEQ ID NO: 7 - DNA, 395 bp, Pyrodictium abyssi
agcttctacgccaccggcacagcacaggcagtaagcgagccaatagacgtggtaagcagcctcggtacgctaaatactgccgctggtgcacagggtaagcagacgctaggagacataacaatatatgcgcacaatgacgtgaacataacaaagctaaaggtcacgcttgctaacgctgcacagctaagaccatacttcaagtacctgataataaagctagtaagcctggacagcaacggcaacgagtccgaggaaaagggcatgataactctatggaagccttacgccgtgataatactagaccatgaagatttcaacaacgacatcgacaatgacggcaacaatgacgccaagataagggttgtagcctactatgaggctaaggagggtatgct

SEQ ID NO: 8 - protein, 131 aa, Pyrodictium abyssi
SFYATGTAQAVSEPIDVVSSLGTLNTAAGAQGKQTLGDITIYAHNDVNITKLKVTLANAAQLRPYFKYLIIKLVSLDSNGNESEEKGMITLWKPYAVIILDHEDFNNDIDNDGNNDAKIRVVAYYEAKEGM

SEQ ID NO: 9 - DNA, 372 bp, Pyrodictium abyssi
agcttctacgccaccggcacagcagaggcaacaagcgagccaatagacgttgtaagcaaccttaacacggccatagcccctgctgccggcgcccagggcagcgtgggcataggcagcataacaatagagaacaagactgacgtgaacgttgtgaagctgaagataaccctcgccaacgctgagcagctaaagccctacttcgactacctacagatagtgctaaagagcgttgacagcaacgagatcaaggctgtgctaagcctcgagaagcccagcgcagtcataatactggacaacgaggacttccagggcggcgacaaccagtgccagatagacgccaccgcctactacgaggctaaggagggtatgcta

SEQ ID NO: 10 - protein, 124 aa, Pyrodictium abyssi
SFYATGTAEATSEPIDVVSNLNTAIAPAAGAQGSVGIGSITIENKTDVNVVKLKITLANAEQLKPYFDYLQIVLKSVDSNEIKAVLSLEKPSAVIILDNEDFQGGDNQCQIDATAYYEAKEGML

SEQ ID NO: 11 - DNA, 448 bp, Artificial Sequence
  consensus sequence
tgagaccctagctgcggattgcctcggctgccgcctcgccctctagcaggcttcgccacacccagagcccctacagcttctacgccaccggcacagcacaggcagtaagcgagccaatagacgtagaaagccacctcacacatagcccctgctgccggcgcacagggcagcaggacataggctacataaaataacaagatagtgaacgtataaagctgaaggtgaccctgctaacgctgagcagctaaagccctacttcaagtacctacagatagtgctaaaagcgacagcaggcacacgagaaggcgtgataagcctcgagaagcctagcgccgtcataatactagacaacgaggacttcgaagcacaacagaaagagaagcaatagcctactacgaggctaaggagggtatgctattcgacagcctcctatataactcaggtctgt

SEQ ID NO: 12 - protein, 140 aa, Artificial Sequence
  consensus sequence
VKTLALAGIIASAALALLAGFATTQSPLSFYATGTAQAVSEPIDVESHLSIAPAAGAQGSDIGYIIKVNVVKLKVTLANAEQLKPYFKYLQIVLSSEIKAVISLDKPSAVIILDEDFAIAYYEAKEGMLFDSLPVINQVL

(19) United States
(12) Patent Application Publication
(10) Pub. No.: US 2008/0274155 A1
(43) Pub. Date: Nov. 6, 2008

(54) Chimeric Cannulae Proteins, Nucleic Acids Encoding Them And Methods For Making And Using Them

(75) Inventors: Nelson R. Barton, San Diego, CA (US); Eileen O'Donoghue, San Diego, CA (US); Ryan Short, Rancho Santa Fe, CA (US); Gerhard Frey, San Diego, CA (US); David Weiner, Del Mar, CA (US); Dan E. Robertson, Belmont, MA (US); Steven Briggs, Del Mar, CA (US); Paul Zorner, Encinitas, CA (US)

Correspondence Address: VERENIUM C/O MOFO S.D., 12531 HIGH BLUFF DRIVE, SUITE 100, SAN DIEGO, CA, 92130-2040, US

(73) Assignee: Verenium Corporation, San Diego, CA (US)

(21) Appl. No.: 10/593,846

(22) PCT Filed: Mar. 24, 2005
(86) PCT No.: PCT/US05/09927

§ 371 Date: Jun. 30, 2008

Related U.S. Application Data
(60) Provisional application No. 60/556,393, filed on Mar. 24, 2004.
(60) Provisional application No. 60/605,192, filed on Aug. 27, 2004.

(51) Int. Cl. A61F 2/00; C07K 14/195; C12N 9/00; C12N 9/40; C12N 9/10; C07H 21/04; C12N 15/63; C12N 1/21; C12N 5/10; C12N 1/19; C12N 1/15; A01K 67/027; A01H 5/00; C12P 1/00; A61K 38/16; A01K 1/015; B32B 9/04; A61K 39/00; A61K 39/395; A61K 31/70; C12N 5/06; A61K 35/12

(52) U.S. Cl. 424/422; 530/350; 435/183; 435/208; 435/193; 536/23.7; 435/320.1; 435/252.3; 435/419; 435/254.2; 435/254.11; 435/348; 800/13; 800/298; 435/41; 514/12; 428/221; 428/704; 424/190.1; 424/139.1; 514/23; 435/395; 424/93.7

(57) ABSTRACT

The invention provides chimeric cannulae polypeptides and nanotubules and methods for making and using them. In one aspect, the invention provides compositions and methods for the identification, separation or synthesis of proteins or ligands. In one aspect, the invention provides compositions and methods for making and using nanotubules. In one aspect, the invention provides compositions and methods for the selection and purification of chiral compositions from racemic mixtures. In one aspect, the chimeric proteins and polymers (e.g., nanotubules, tubules, bundles, balls, fibers, filaments, sheets, threads, textiles) of the invention comprise a detectable moiety, e.g., a fluorescent protein. In one aspect, the invention provides a flame retardant or heat resistant device comprising a sheeting, a covering, a coating or an adhesive comprising a chimeric protein of the invention.

### TECHNICAL FIELD

This invention relates to nanotechnology, pharmacology and drug synthesis. In one aspect, the invention provides compositions and methods for the identification, separation or synthesis of proteins or ligands. In one aspect, the invention provides compositions comprising polypeptides of the invention assembled into bundles, filaments, threads, sheets or nanotubules, and methods for making and using these nanotubules. In one aspect, the chimeric proteins and nanotubules of the invention comprise a detectable moiety, e.g., a fluorescent protein. In one aspect, the invention provides compositions and methods for the selection and purification of chiral compositions from racemic mixtures. In one aspect, the invention provides chimeric cannulae polypeptides and methods for making and using them.

### BACKGROUND

Enantiomers frequently display dramatically different pharmacological properties. As a result, use of single-enantiomer drugs may improve efficacy and reduce side effects. The United States Food and Drug Administration also recognizes the importance of understanding the pharmacological properties of each enantiomer. In order for a racemic drug to be registered, the biological activity of each purified enantiomer must be characterized.

Cannulae A, or CanA, is a heat-resistant protein capable of forming nanotubules. CanA nanotubules are assembled from 21 kDa monomeric subunits that self-assemble in the presence of divalent cation into hollow rods with an outer diameter of approximately 25 nm and an inner diameter of approximately 20 nm, thus exhibiting molecular dimensions and an overall morphology not dissimilar to eukaryotic microtubules. CanA monomer expressed in E. coli is heat-stable. It can be rapidly purified from bacterial extracts following heat treatment to remove the majority of the heat-labile host proteins. Following purification, the CanA monomer readily self-assembles into nanotubules in the presence of calcium and magnesium at elevated temperature. The assembled nanotubule structure contains 28 CanA monomers per turn arranged with a helical pitch. The CanA nanotubules are heat stable (up to 128° C.) and remain assembled in the presence of SDS or high concentrations of urea. See, e.g., Short, et al., WO 02/44336.

Cannulae nanotubules are characteristically formed by Pyrodictium abyssi, a hyperthermophilic microorganism discovered in a high temperature environment (>100° C.). In its natural environment and in cell culture, Pyrodictium abyssi are linked together by a meshwork of these nanotubular fibers that both connect and entrap the cells. These fiber networks are a unique feature of the genus Pyrodictium and they appear to be required for growth above 100° C. In addition, there appears to be a direct association between the maintenance of these nanotubular connections and cellular growth as demonstrated by the observation that, at the onset of cellular fission, these nanotubules appear to form loops attached at both ends to the growing cell. Following cellular fission the nanotubular loops become links connecting daughter cells. While it remains speculative as to what the true role of the nanotubules is in nature, it has been suggested that the linkage of cells by these tubules could enable cells to exchange metabolites, genetic information, or signal compounds.

Current flame retardant technology relies upon the application of chemical retardants to refined cotton. Though flame retardancy is a key issue in clothing, auto and home upholstery, carpeting and many other applications, the current and historical means for providing the characteristic to cotton have resulted in environmental persistence of the applied chemicals. These chemicals, polybrominated diphenyl ethers (PBDE), have been shown to appear in breast milk and in soils.

Fiber ignition, flame flowing, and persistence of glow in cotton is a complex phenomenon which has been explained by coating, gas, thermal and chemical theories. Fireproofing and flame resistance in cotton has been approached in a number of ways. The most common current solution is post-production coating of materials with halogenated compounds. These compounds, which bind by either esterification or etherification to cellulose, produce a surface foam upon ignition and prevent spread of flame or glow in the fabric.

### SUMMARY

The invention provides chimeric polypeptides comprising at least a first domain comprising a cannulae polypeptide and at least a second domain comprising a heterologous polypeptide or peptide, carbohydrate, small molecule, nucleic acid or lipid. The heterologous polypeptide or peptide can be inserted at the amino terminal end, the carboxy terminal end or internal to the cannulae polypeptide, or, if the cannulae polypeptide comprises more than one heterologous polypeptide or peptide, a mixture thereof. The cannulae polypeptide can comprise a protein having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more, or complete (100%) sequence identity to SEQ ID NO:2, SEQ ID NO:4, SEQ ID NO:6, SEQ ID NO:8, SEQ ID NO:10 or SEQ ID NO:12. In alternative aspects, the polypeptide monomers of the invention are capable of assembling into a polymer, e.g., a nanotubule, bundle, ball, filament, thread, or sheet, or, are capable of acting as chiral selectors. In one aspect, the polypeptide polymers having a sequence as set forth in SEQ ID NO:2, SEQ ID NO:4, SEQ ID NO:6, SEQ ID NO:8, SEQ ID NO:10 or SEQ ID NO:12 are designated NANODEX™ polymers. The chimeric cannulae proteins can assemble into nanotubular polymers to act as chiral selectors, biosynthetic pathways, selection scaffoldings, scaffolds for the construction of multi-enzyme heteropolymers (e.g., biosynthetic pathways), and the like.

The second domain of the heterologous polypeptide or peptide of the chimeric polypeptides of the invention can comprise any moiety, e.g., an enzyme, a binding protein (e.g., an antigen binding site, such as an antibody, a binding protein, such as biotin, an enzyme, a cytokine, and the like). In one aspect, a monomer, and thus a polymer (e.g., a nanotube, bundle, filament, thread or sheet) can also comprise any functionalized group, which can be directly bound to a monomer or polymer of the invention, or indirectly bound, e.g., a ligand bound to an antibody or an avidin to a biotin, where the antibody or biotin are the heterologous polypeptide or peptide of the polypeptide of the invention. The avidin can be further conjugated to any moiety, the resulting polymer can be fulinctionalized with any composition, including small molecules, metal ions, mono- or polysaccharides, lipids, nucleic acids, and the like. Thus, the invention provides a “biofunctional” polymer, which, in one aspect, can be spun into a fiber, which in turn can be used to make a “biofunctional” textile, fabric, sheeting, covering, coatings, adhesive, and the like.

For example, in one aspect, polypeptide polymers of the invention (e.g., a nanotube, bundle, filament, thread or sheet of the invention) are used as flame (fire) or heat retardants and can be incorporated into any material, e.g., fabrics (which can be designated NANOAVID™ textiles), fibers, adhesives and the like. In one aspect, polypeptide polymers of the invention are used to imbue a heat resistance characteristic on a material, e.g., to make a material more heat resistant.

In one aspect, the cannulae polypeptide is capable of assembling into a polymer, such as a nanotubule, bundle, ball, filament, thread or sheet. In one aspect, the cannulae polypeptide is capable of self-assembling into a polymer. In some aspects, the monomers require a co-factor for polymer assembly, e.g., a divalent cation, or, a “nucleation factor,” which can be another cannulae monomer. The divalent cation can be Ca²⁺, Mg²⁺, Cu²⁺, Zn²⁺, Sr²⁺, Ni²⁺, Mn²⁺ and/or Fe²⁺. In another aspect, both Ca²⁺ and Mg²⁺ are needed for polymer assembly, e.g., into nanotubules, bundles, filaments, threads or sheets. In one aspect, divalent cation(s) are in millimolar concentrations during polymer assembly.

In one aspect, the heterologous polypeptide or peptide is expressed in the inner lumen of a nanotubule or on the exterior of the nanotubule. These hybrid nanotubules can array the heterologous polypeptides or peptides on the outer surface or the inner luminal surface of a tubular polymer, or, when a monomer comprises more than one heterologous peptide or protein, they can be “displayed” on both the outer and inner surfaces of the tubules. If all the monomers of a nanotubule comprise a heterologous polypeptide or peptide in a similar manner, then that heterologous polypeptide or peptide can be displayed in a regular helical pattern on the nanotubule.

In one aspect, the heterologous polypeptide or peptide comprises a chiral selection motif, a receptor or a ligand, an enzyme, an enzyme active site, a cofactor, a substrate, an antigen or an antigen binding site, a detectable moiety, e.g., a green fluorescent protein, an alpha-galactosidase or a selection factor, e.g., a chloramphenicol acetyltransferase.

In one aspect, the chimeric polypeptide is a recombinant protein, which can be expressed in vitro or in vivo, a synthetic protein, or a mixture thereof.

In one aspect, at least one subsequence of the cannulae polypeptide domain of a chimeric protein of the invention has been removed. A heterologous polypeptide or peptide can be inserted into the cannulae polypeptide at the site (or one of the sites) subsequence(s) were removed. In one aspect, the cannulae polypeptide is a CanA polypeptide and the removed subsequence is a 14 residue motif (peptide) consisting of residue (position) 123 to residue 136 of SEQ ID NO:2 (i.e., “PDIKTGYTNTSIVP”), or, a 17 residue motif (peptide) located at amino acid residue (position) 123 to residue 139 of SEQ ID NO:2, (i.e., “PDKTGYTNTSIWVPGEP”). The heterologous polypeptide or peptide can be inserted into the CanA polypeptide at one or both of the sites of the 14 or 17 residue motif subsequences that were removed. The heterologous peptide can be a 14 residue or a 17 residue peptide inserted into the CanA polypeptide to replace the removed 14 residue or 17 residue motif. Alternatively, the heterologous peptide can be shorter, or, longer. Heterologous peptides can also be attached (e.g., recombinantly, or, by linker) to either end of a CanA polypeptide.

The invention provides immobilized chimeric polypeptides comprising a chimeric monomeric or polymeric polypeptide of the invention. The invention provides polymers, e.g., nanotubules, comprising a plurality of chimeric polypeptides of the invention. In one aspect, the polymer is a heteropolymer, e.g., a nanotubule assembled from more than one cannulae polypeptide, including monomers other than the chimeric proteins of the invention, or other polypeptides or compositions. The heterologous polypeptide or peptide comprises an enzyme, e.g., an active site, or a plurality of different enzymes. The plurality of enzymes can comprise a biosynthetic pathway. The plurality of enzymes can be arranged along the length of the nanotubule in the same order as they act in the biosynthetic pathway. In one aspect, the scaffolding of the plurality of enzymes acts as an “array-type” solid support.

The different enzymes comprising the biosynthetic pathway can be separated from each other along the length of the tubule by cannulae monomers lacking a heterologous protein or peptide (e.g., a “wild type” cannulae monomer, such as CanA, CanB, CanC, CanD, CanE and the like). The polymers comprising a biosynthetic pathway can also comprise substrate(s), co-factor(s), regulatory agents and the like.

The invention provides polymers, e.g., nanotubules, wherein the heterologous polypeptide or peptide comprises at least one chiral selection motif, such as an enzyme or an enzyme active site.

The invention provides nucleic acids comprising a sequence encoding a chimeric polypeptide of the invention. The invention provides expression cassettes (e.g., vectors, recombinant viruses, phages, etc.) comprising a sequence encoding a chimeric polypeptide of the invention. The invention provides cells comprising a sequence encoding a chimeric polypeptide of the invention, or, an expression cassette of the invention. The cell can be any cell, e.g., a bacterial cell, a plant cell, a yeast cell, a fungal cell, an insect cell or a mammalian cell. The invention provides transgenic non-human animals comprising a sequence encoding a chimeric polypeptide of the invention, or, an expression cassette of the invention. The invention provides plants comprising a sequence encoding a chimeric polypeptide of the invention, or, an expression cassette of the invention.

The invention provides methods for the chiral selection of a composition, comprising the following steps: providing a chimeric polypeptide of the invention; providing a racemic mixture of the composition; and, contacting the racemic mixture with the chimeric polypeptide under conditions wherein only one enantiomer of the composition binds to the chimeric polypeptide; thereby selecting a single chiral specie of the racemic mixture. The invention provides methods for the chiral selection of a composition, comprising the following steps: providing a nanotubule of the invention; providing a racemic mixture of the composition; and, contacting the racemic mixture with the nanotubule under conditions wherein only one enantiomer of the composition binds to the nanotubule; thereby selecting a single chiral specie of the racemic mixture. The methods further comprise separation of the different chiral species.

The invention provides methods for enzymatic biosynthesis of a composition, comprising the following steps: providing a nanotubule, bundle, ball, filament, thread or sheet of the invention comprising a plurality of enzymes comprising a biosynthetic pathway; providing a substrate for, at least one enzyme; and, contacting the nanotubule, bundle, filament, thread or sheet with the substrate under conditions wherein the enzymes of the biosynthetic pathway catalyze the synthesis of the composition. In one aspect, the enzymes are expressed in the inner lumen of the nanotubule, or, they are expressed on the exterior of the nanotubule. The nanotubules can also comprise substrates(s), co-factor(s), regulatory factors, cytokines, carbohydrate, cell or cell matrix binding domain (e.g., stem cell binding domain), carbohydrates, small molecules, lipids, nucleic acids, metals, metal chelating agents, and the like.

The invention provides pharmaceutical compositions comprising a chimeric polypeptide of the invention or a tubule or nanotubule, bundle, ball, fiber, filament or sheet of the invention. The invention provides pharmaceutical compositions comprising a chimeric polypeptide comprising at least a first domain comprising a cannulae polypeptide and at least a second domain comprising a heterologous domain. In one aspect, the heterologous domain is attached at the amino terminal end, the carboxy terminal end or internal to the cannulae polypeptide. In one aspect, the cannulae polypeptide comprises a protein having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity to SEQ ID NO:2, SEQ ID NO:4, SEQ ID NO:6, SEQ ID NO:8, SEQ ID NO:10 or SEQ ID NO:12. In one aspect, the chimeric polypeptide comprises a recombinant fusion protein and the heterologous domain comprises polypeptide or a peptide.

In one aspect, the heterologous domain of the chimeric polypeptide comprises an epitope, an immunogen, a toleragen, a carbohydrate binding domain, a cell or cell matrix binding domain (e.g., stem cell binding domain), a small molecule, a small molecule binding domain, a lipid, a carbohydrate, a cytokine, an enzyme or an antibody.

The invention provides vaccines comprising a chimeric polypeptide of the invention or a tubule or nanotubule, bundle, ball, fiber, filament or sheet of the invention, and a pharmaceutically acceptable excipient. The heterologous polypeptide or peptide of the chimeric polypeptide can comprise an epitope, an immunogen, a toleragen, an immunomodulatory agent, an immune suppression agent, an adjuvant, an antibody, a cell binding agent, a lipid, a carbohydrate or a combination thereof. In one aspect, the chimeric polypeptide is assembled or self-assembles into a tubule or nanotubule, bundle, ball, fiber, filament or sheet. The vaccine can be formulated as a liquid, a powder, a spray, an implant, a tablet, a pill or a capsule.

The invention provides methods for modulating the immune system of an individual comprising administering a pharmaceutically effective amount of a composition of the invention, a pharmaceutical composition of the invention. In one aspect, a humoral (antibody) or a cell-based (e.g., helper T cell or cytotoxic T cell) immune response is elicited in the individual. In one aspect, modulating the immune system comprises suppressing a new immune response, abrogating or diminishing a new or recurrent immune response (e.g., an allergic or autoimmune response), or a tolerizing response. In one aspect, the individual is a human.

The invention provides carbohydrate-based therapeutic pharmaceutical comprising a composition of the invention, or a tubule or nanotubule, bundle, ball, fiber, filament, thread, or sheet of the invention, wherein the composition, tubule or nanotubule, bundle, ball, fiber, filament, thread, or sheet comprises at least one carbohydrate. In the carbohydrate-based therapeutic pharmaceutical the composition, tubule or nanotubule, bundle, ball, fiber, filament, thread, or sheet can comprise a polypeptide or peptide having a carbohydrate-binding motif. In one aspect, the carbohydrate-binding motif is an N-linked carbohydrate-binding motif or an O-linked carbohydrate-binding motif. In one aspect, the carbohydrate is added chemically, by cellular biosynthetic mechanisms, by in vitro enzymatic reactions, or a combination thereof.

The invention provides methods for ameliorating a disease or condition comprising administering a pharmaceutically effective amount of a carbohydrate-based therapeutic pharmaceutical composition of the invention to an individual. In one aspect, ameliorating the disease or condition comprises inhibition of carbohydrate-lectin interactions; immunization with carbohydrate antigens; inhibition of enzymes that synthesize disease-associated carbohydrates; inhibition of carbohydrate-processing enzymes; targeting of drugs to specific disease cells via carbohydrate-lectin interactions; administering carbohydrate based anti-thrombotic agents.

The invention provides cell matrix binding compositions comprising a composition of the invention, or a tubule or nanotubule, bundle, ball, fiber, filament, thread, or sheet of the invention, wherein the composition, tubule or nanotubule, bundle, ball, fiber, filament, thread, or sheet comprises at least one a cell matrix binding motif. The cell matrix binding motif can comprise an RGD-binding motif or an RGD motif. The cell matrix binding composition can comprise a medical device. The cell matrix binding composition of claim 88, wherein the medical device can comprise a dental or orthopedic prostheses, a dental device or implant, an orthopedic device, a pin, a screw, a fixture, a plate, a stent, a stent sheath, a shunt, a catheter, a valve, a cannulae, a tissue scaffold, a wound care device, a dressing or a lens.

The invention provides tissue scaffolds or implant materials comprising a composition of the invention, or a tubule or nanotubule, bundle, ball, fiber, filament, thread, or sheet of the invention. The tissue scaffold can comprise a polymer scaffold and neural stem cells for repairing a spinal cord injury. The tissue scaffold can comprise can comprise a vascular graft comprising graft material from smooth muscle, endothelial muscle and/or stem cells.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is an illustration of a transmission electron micrograph of nanotubules assembled from recombinant CanA expressed in E. coli.

FIG. 2 is a schematic representation of the open reading frames of the CanA and CanB sequences, showing the CanA sequence containing a 14 amino acid domain not found in CanB.

FIG. 3 is an illustration of an immunofluorescent light microscope image of nanotubules assembled from a fusion protein generated by fusing the CanA open reading frame (SEQ ID NO:1) to the open reading frame of the green fluorescent protein ZSGREEN™.

FIG. 4 is an illustration an exemplary process for constructing a heteropolymer of the invention generated by self-assembly of different chimeric monomers, as described below.

Like reference symbols in the various drawings indicate like elements.

### DETAILED DESCRIPTION

The invention provides compositions (including chimeric proteins and nanotubules) and methods for use in all aspects of nanotechnology. The compositions of the invention, e.g., chimeric proteins and nanotubules, bundles, filaments, threads or sheets comprising a cannulae protein (e.g., a protein of the invention), can be used in any biological or synthetic system. For example, the chimeric proteins and polymers (e.g., nanotubules, bundles, filaments, threads or sheets) of the invention can be used in electronic devices, such as circuits, transistors, memory storage devices, devices for current conduction, or any aspect of a computer, transmitter, detector and the like. The chimeric proteins and polymers (e.g., nanotubules, bundles, filaments, thread or sheets) of the invention can be used in any pharmaceutical, medical device, artificial organ, prosthesis, implant, and the like, for example, as a structural element or a coating. The chimeric proteins and polymers (e.g., nanotubules, bundles, filaments, thread or sheets) of the invention can be used in any article of manufacture, e.g., for biosynthetic scaffolding, camouflage or as heat resistance structural elements, e.g., in fabrics, fibers or any material. In one aspect, polypeptide polymers of the invention are used as flame retardants or preventatives and can be incorporated into any material, e.g., fabrics (which can be designated NANOAVID™ textiles), fibers, adhesives and the like. In one aspect, polypeptide polymers of the invention are used to imbue a heat resistance characteristic to a material, e.g., to make a material more heat resistant.

In one aspect, the chimeric proteins or polymers (e.g., nanotubules, bundles, filaments, thread or sheets) of the invention comprise a detectable moiety, e.g., a fluorescent or luminescent protein or other moieties, a radioactive moiety, an epitope and the like; or, an enzyme, which can by its catalytic activity generate a detectable moiety, for example, beta galactosidase. In one aspect, inclusion of a fluorescent or luminescent protein or other moiety in a product of manufacture of the invention allows detection of wavelengths. Thus, the invention provides products of manufacture comprising chimeric proteins or polymers (e.g., nanotubules, bundles, filaments, thread or sheets) of the invention and moiety capable of detecting fluorescence or luminescence, and, in one aspect, the products of manufacture act as a wavelength-specific reconnaissance device.

The invention provides compositions and methods for the identification, separation or synthesis of proteins or ligands using chimeric cannulae polypeptides (i.e., fusion, or hybrid, proteins). Chimeric cannulae polypeptides of the invention include CanA fusion proteins comprising SEQ ID NO:2 (encoded, e.g., by SEQ ID NO:1), CanB fusion proteins comprising SEQ ID NO:4 (encoded, e.g., by SEQ ID NO:3), CanC fusion proteins comprising SEQ ID NO:6 (encoded, e.g., by SEQ ID NO:5), CanD fusion proteins comprising SEQ ID NO:8 (encoded, e.g., by SEQ ID NO:7), CanE fusion proteins comprising SEQ ID NO:10 (encoded, e.g., by SEQ ID NO:9), or the cannulae polypeptide comprising the consensus sequence SEQ ID NO:12 (encoded, e.g., by SEQ ID NO:11), or subsequences thereof.

In one aspect, the compositions and methods are used for the chiral separation of proteins and other compositions. For example, cannulae (e.g., CanA, CanB, CanC, CanD, CanE) fusion proteins can be used as chiral separations material. The chimeric cannulae polypeptides of the invention can be used as chiral separation materials in monomer or polymer (e.g., nanotubule) forms. When used in nanotubule forms, the motif of the cannulae polypeptide responsible for chiral selectivity can be exposed to the inner lumen of the tubule or on the outer surface of the tubule, or both.

The invention provides cannulae chimeric proteins, e.g., recombinant fusion proteins (chimeric monomers) comprising a cannulae polypeptide (e.g., CanA, CanB, CanC, CanD, CanE) further comprising a heterologous polypeptide or peptide. The heterologous polypeptide or peptide can be an enzyme, an enzyme active site, a ligand, a receptor, an antigen, an epitope (e.g., a T cell epitope or a B cell epitope), an antibody, a heat shock protein domain, an N- or O-linked glycosylation site, a nucleic acid binding protein, a cell matrix binding motif (e.g., an RGD motif or other integrin binding motif) and the like. The heterologous polypeptide or peptide can be any sequence for the chiral selection of a protein or other composition. For example, a chiral selection heterologous polypeptide or peptide can be an enzyme or an enzyme active site motif.

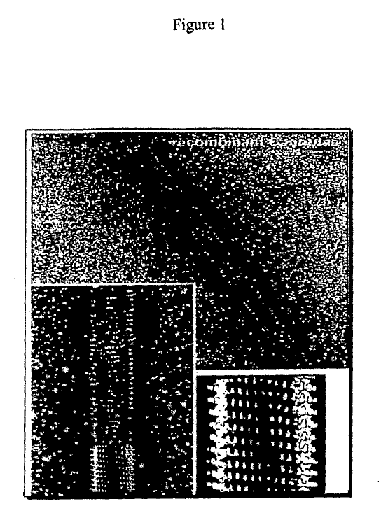

In one aspect, the cannulae fusion proteins are monomeric or polymeric, e.g., dimers, trimers, etc., or nanotubules, bundles, filaments, threads or sheets, as illustrated in FIG. 1. Cannulae chimeric polymers, e.g., nanotubules, can act as high density preparation materials, e.g., where the heterologous polypeptide or peptide comprises a chiral selection motif.

Cannulae chimeric polymers, e.g., nanotubules, bundles, filaments, thread or sheets, also can act as a high density selection materials, e.g., where the heterologous polypeptide or peptide comprises a receptor, an enzyme, a ligand, an epitope (e.g., a T cell epitope or a B cell epitope), an antibody, a heat shock protein domain, an N- or O-linked glycosylation site, a nucleic acid binding protein, a cell matrix binding motif and the like. In aspects where the cannulae chimeric polymers form as nanotubules, the heterologous polypeptide or peptide can be expressed on the outer surface of the nanotubule, on the inner surface of the tubule's lumen, or both. Positioning of the fusion partner on the exterior or interior of the assembled protein nanotubule can lead to changes in the half-life of the fusion domain such that surface-displayed fusion domains may be digested more rapidly by host proteases than interior-facing (less accessible, more shielded) fusion domains.

In alternative aspects, the heterologous polypeptide or peptide is fused to the N-terminus of the cannulae protein (e.g., CanA), fused into a loop domain (e.g., of CanA), or fused to the C-terminus of the cannulae protein (e.g., CanA).

Cannulae chimeric polymers, e.g., nanotubules, bundles, filaments, threads or sheets, also can act as a biosynthetic scaffolding, e.g., where nanotubules of the invention comprise a plurality of heterologous polypeptide or peptides in the form of enzymes, catalytic antibodies or enzyme active sites comprising a biosynthetic pathway. In one aspect, the enzymes, catalytic antibodies or enzyme active sites are all expressed on one surface of a polymer, e.g., a nanotubule, e.g., on the outer surface or on the inner lumen of the tubule. In one aspect, the enzymes, catalytic antibodies or enzyme active sites are arranged along the length of the tubule (in the interior or exterior of the tubule) in the same order of their action in the biosynthetic pathway. Any number of enzymes, catalytic antibodies or enzyme active sites can be immobilized onto or into a tubule. Any biosynthetic pathway can be reconstructed along a nanotubule of the invention.

In one aspect, the cannulae chimeric proteins, e.g., recombinant fusion proteins (chimeric monomers) comprising a cannulae polypeptide (e.g., CanA, CanB, CanC, CanD, CanE) are used in vaccines, e.g., to elicit an immune response, to initiate an immune response, to modulate an immune response, to suppress immune response, to monitor an immune response. The cannulae chimeric proteins of the invention can be used as the immunizing reagent alone or with other compositions, e.g., an adjuvant. In one aspect, the invention provides methods to build fusion monomers from cannulae polypeptides (e.g., CanA, CanB, CanC, CanD, CanE) that incorporate a one or a combination of antigenic epitopes and immunomodulatory domains (T-cell epitopes, B-cell epitopes, heat shock protein domains, enzymes, cytokines, carbohydrates, etc.). Fusion monomers containing one or more of these fusion-partner protein domains can be pooled in varying ratios and then incubated under conditions that drive self-assembly of the monomers into polymer. In one aspect, an assembled polymer of the invention is a heteropolymer comprising one or more antigenic determinants and one or more immunomodulatory domains. In alternative aspects, the heterologous protein or peptide is fused to the N-terminus of CanA, fused into a loop domain of CanA, or fused to the C-terminus of CanA. In one aspect, the fusion partners are displayed on an interior surface, an external surface, or both, of an assembled tubular polymer. In one aspect, the fusion partner is positioned on the exterior or interior of a nanotubule to manipulate the half-life of the fusion domain. Surface-displayed fusion domains are digested more rapidly by host proteases than interior-facing (less accessible, more shielded) fusion domains.

In one aspect, nanotubules, bundles, filaments, threads or sheets comprising a plurality of enzymes, catalytic antibodies and/or enzyme active sites are generated by constructing a cannulae polypeptide-enzyme fusion protein by fusing the open reading frame of a cannulae polypeptide (e.g., CanA, CanB, CanC, CanD, CanE) to the open reading frame of a desired enzyme sequence using standard molecular cloning techniques. The fusion sequence is then cloned into an appropriate expression cassette, e.g., an over-expression vector, prokaryotic or eukaryotic, and expressed as recombinant proteins. Expressed fusion protein can be purified from host proteins before polymer assembly. For example, chimeric proteins (e.g., monomers) can be purified by heat treatment to denature heat-labile host proteins (e.g., at about 80 to 100° C., for about 2 to 20 minutes). The soluble heat-stable fusion protein can be further purified from contaminating proteins by other conventional means, e.g., chromatography techniques, e.g., ion exchange chromatography, HPLC and the like.

Purified, partially purified or unpurified chimeric (fusion) proteins can be induced to assemble into nanotubules by heating the fusion monomer solution (e.g., to about 80° C.) in the presence of millimolar concentrations of a bivalent cation, e.g., calcium and/or magnesium. The polymer can be collected, e.g., by centrifugation (e.g., at 30,000×g for 30 minutes), chromatography and the like.

The invention provides heteropolymers, e.g., nanotubules, bundles, filaments, threads or sheets, comprising any variety of compositions, such as enzymes, catalytic antibodies and/or enzyme active sites, co-factors, substrates and the like. In one aspect, heteropolymers of the invention are constructed to act as a biosynthetic pathway(s) along the length of the polymer (e.g., nanotubule). In one aspect, heteropolymers of the invention are constructed to act as a biosynthetic pathway and a chiral selection mechanism. Heteropolymers (e.g., nanotubules, bundles, filaments, threads or sheets) of the invention can also comprise any variety of antibodies, antigens, receptors, ligands, binding sites and the like, spatially arranged in any desired manner along the length of the polymer.

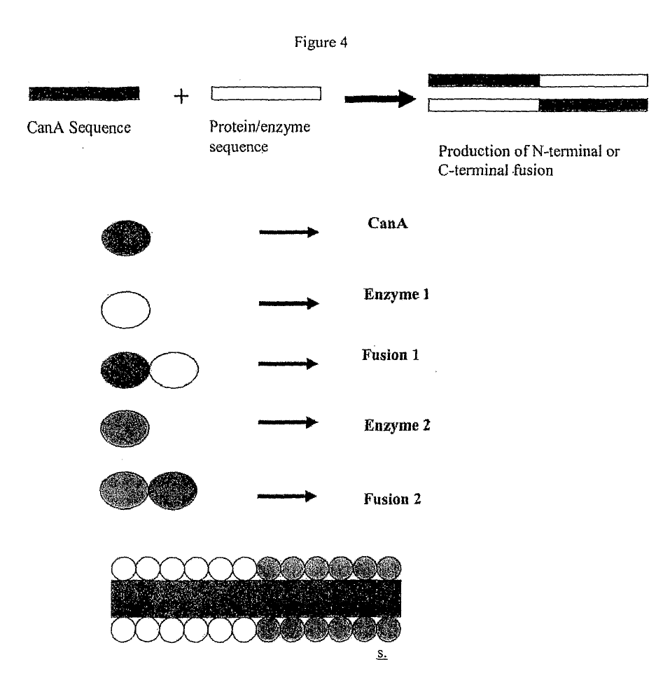

Heteropolymers (e.g., nanotubules, bundles, filaments, threads or sheets comprising a plurality of different enzymes, catalytic antibodies and/or enzyme active sites comprising a biosynthetic pathway) can be constructed by an exemplary protocol as illustrated in FIG. 4. Nucleic acids encoding chimeric monomers are constructed and expressed. The heterologous protein or peptide can be inserted at the amino terminal, carboxy terminal (as shown in FIG. 4) or internal to the cannulae polypeptide (e.g., CanA, CanB, CanC, CanD, CanE). One or more, or all, or the expressed chimeric monomers can be purified. Self-assembly of the heteropolymer can be initiated with one of the chimeric polypeptides, e.g., fusion 1 monomer pool as shown in FIG. 4. Next, in this exemplary protocol, fusion 1 polymer is rapidly diluted with fusion 2-monomer pool such that the majority of the subunits added to the growing polymer are fusion 2 monomers. Alternatively, unassembled fusion 1 monomers are removed and fusion 2 monomers added. The resulting polymer is composed of a length of fusion 1 and a length of fusion 2 monomer. This process can be iteratively repeated until a nanotubule of a desired length comprising a desired number of different enzymes, catalytic antibodies and/or enzyme active sites comprising a biosynthetic pathway is generated. The resulting nanotubule can serve as a scaffold for the assembly of an oriented, multi-enzyme complex.

In alternative aspects, the invention provides heteropolymers comprising different ratios of fusions and wild-type, non-fusion monomers to assemble nanotubular polymers that display one or more enzyme (or other, e.g., binding or co-factor) activities, at controlled loading, e.g., on the exterior or interior surface, or both, of a nanotubule.

In one aspect, any number of compositions desired to be immobilized along the length of a polymer of the invention (e.g., nanotubules, bundles, filaments, threads or sheets), whether a protein or a non-protein composition, e.g., enzymes, catalytic antibodies, enzyme active sites, co-factors (e.g., NADH, FADH, ATP and the like), substrates, antibodies, antigens, receptors, ligands, binding sites and the like, can be spatially arranged in any desired manner along the length of the polymer by indirect immobilization to the polymer. In this aspect, immobilization agents (e.g., a receptor, an enzyme, a ligand, an epitope (e.g., a T cell epitope or a B cell epitope), an antibody, a heat shock protein domain, an N- or O-linked glycosylation site, a carbohydrate, a nucleic acid binding protein, and the like), a cell matrix binding motif, are arranged as desired along the interior or exterior, or both, length of the polymer. The composition to be immobilized can be constructed to include (e.g., a chimeric recombinant protein) or be complexed with a moiety that will bind to an indirect immobilization agent. The indirect immobilization agent can be a binding agent for the composition to be immobilized. For example, a nanotubule is constructed having ten different antibodies spatially arranged along the length of the tubule. This nanotubule can be constructed by a method analogous to that illustrated in FIG. 4, e.g., instead of chimeric monomers comprising enzymes, the chimeric monomers would comprise antibodies (including, e.g., antigen binding sites) that specifically bind to different, desired enzymes, substrates, co-factors and the like.

In one aspect, the chimeric cannulae proteins of the invention self-assemble into helical nanotubular protein polymers. These helical nanotubular protein polymers can act as a chiral selectors, biosynthetic pathways, selection scaffoldings and the like. These hybrid protein nanotubules can array the heterologous polypeptide or peptide (fusion partner) on the outer surface or the inner luminal surface of a tubular polymer. If all the monomers of a nanotubule comprise a heterologous polypeptide or peptide in a similar manner, then that heterologous polypeptide or peptide can be displayed in a regular helical pattern on the nanotubule.

In addition to serving as chiral selectors, biosynthetic pathways, selection scaffoldings, etc. comprising chimeric monomers of the invention, polymers of the invention (e.g., nanotubular protein polymers, bundles, filaments, threads or sheets) can also comprise unmodified cannulae monomers, modified non-chimeric cannulae monomers or other polypeptides. For example, in one aspect, a nanotubule of the invention comprises a chimeric monomer A, an unmodified cannulae monomer, a chimeric monomer B, etc. Inclusion of unmodified cannulae monomers can provide “spacing” between the “clusters” of heterologous peptides or polypeptides expressed on the inner or outer surface of a nanotubules (spatially arranged, e.g., as illustrated in FIG. 4). In one aspect, a polymer of the invention is designed to comprise a mix of proteins having different stabilities under different conditions, e.g., a nanotubule comprising temperature stable and temperature labile monomers (chimeric or wild type, e.g., SEQ ID NO:2, SEQ ID NO:4, SEQ ID NO:6, SEQ ID NO:8, SEQ ID NO:10, or, the consensus cannulae sequence, SEQ ID NO:12). In one aspect, a polymer of the invention is designed to comprise a mix of different proteins, e.g., cannulae polypeptides, including chimeric, wild type or otherwise modified, e.g., non-thermostable.

In one aspect, a subsequence of a cannulae protein is removed and replaced by the heterologous polypeptide or peptide, or, the heterologous polypeptide or peptide can be added to a cannulae monomer. The removed subsequence can be amino- or carboxy-terminal, or, it can be internal to the cannulae protein. In one aspect, the removed subsequence is a motif that is expressed on the inner surface and/or the exterior surface of a cannulae nanotubule. Thus, when the removed sequence is expressed by a heterologous sequence, the heterologous sequence is also expressed on the inner or the outer surface (or both) of the tubule.

In one aspect, for the fusion (hybrid) CanA protein, the removed subsequence consists of a 14 residue motif consisting of residue 123 to residue 136 of SEQ ID NO:2 (i.e., “PDKTGYTNTSIWVP”), or, a 17 residue motif located at amino acid residue 123 to residue 139 of SEQ ID NO:2, (i.e., “PDKTGYTNTSIWVPGEP”). In one aspect, the removed sequence is replaced by a heterologous polypeptide or peptide. When the CanA monomer is in polymeric nanotubular form, a 14 residue motif consisting of residue 123 to residue 136 of SEQ ID NO:2 (i.e., “PDKTGYTNTSIWVP”), or, a 17 residue motif located at amino acid residue 123 to residue 139 of SEQ ID NO:2, (i.e., “PDKTGYTNTSIWVPGEP”) is expressed on the outer surface of the nanotubule. In this aspect, the CanA monomer protein can act as a chiral selector on the outer surface. If all the monomers of a nanotubule comprise a heterologous polypeptide or peptide inserted in or near this motif position (as an addition or a full or partial replacement for the CanA motif), then that heterologous polypeptide or peptide can be displayed in a regular helical pattern on the outer surface of a CanA nanotubule. In one aspect, a 14 residue or a 17 residue heterologous peptide replaces the removed 14 residue motif consisting of residue 123 to residue 136 of SEQ ID NO:2 or the 17 residue motif located at amino acid residue 123 to residue 139 of SEQ ID NO:2.

In one aspect, the chimeric cannulae protein of the invention, either in monomeric or polymer (e.g., nanotubules, bundles, filaments, threads or sheets) form, are stable to a variety of conditions, e.g., temperature, pHs, chaotropic agents, detergents and the like. In one aspect, a polymer of the invention comprises is a heteropolymer comprising monomers of different stabilities under different conditions.

In one aspect, the monomers and polymers of the invention are used as chiral selectors, and methods for using these compositions for the chiral selection of compositions from racemic mixtures. The net charge and electrophoretic mobility of a protein chiral selector can be directly affected by the pH of the buffer solution (e.g., aqueous buffers) used during the separation. In one aspect, the separation methods of the invention (e.g., the chiral separation methods using cannulae fusion (hybrid) proteins as a chiral selectors) are practiced over a range of pH values. The pH of the buffer solution for use in the separations methods can be varied and optimal pH can be determined by routine screening. In one aspect, the methods are practiced over an operating range from about pH 5.5 to 8.5, or, pH 3 to pH 10, or, pH 2.5 to pH 11.

In one aspect, the separations methods of the invention (e.g., chiral selections) are practiced over a range of pH values and in the presence of SDS and/or urea. The presence of SDS and/or urea can improve aqueous chiral separations; see, e.g., Bojarski (1997) Electrophoresis 18:965-969. The stability screenings can be conducted as follows: purified recombinant cannulae monomer protein is assembled into polymer using an in vitro assembly protocol at neutral pH. Following completion of the assembly reaction, the sample is centrifuged and pelleted cannulae polymer collected.

The stability of nanotubules comprising cannulae fusion (hybrid) proteins can be affected by the buffer environment used in practicing the methods of the invention. The separation methods of the invention (e.g., the chiral separation methods) can be practiced in a variety of commonly used organic modifiers. In one aspect, organic modifiers are added to buffers used in practicing the methods of the invention to improve the resolution of enantiomers. The concentration of modifiers for use in the separations methods can be varied and optimal concentrations can be determined by routine screening.

In one aspect the invention provides methods to evaluate the stability of the polymers of the invention in the presence of commonly used organic modifiers, e.g., as listed in the following table:

| Organic Modifier | Concentration Range |
|---|---|
| Methanol | 0-15% |
| Ethanol | 0-15% |
| 1-propanol | 0-15% |
| 2-propanol | 0-15% |
| acetonitrile | 0-15% |

These modifiers are organic modifiers commonly used in protein-based chiral selection methods development. The methods of the invention incorporate these and other organic modifiers and protocols as discussed by, e.g., Busch (1993) J. of Chromatography A. 635:119-126; De Lorenzi (1997) J. of Chromatography A. 790:47-64; Ahmed (1997) J. of Chromatography A. 766:237-244. All of the analytical methods used for the evaluation of polymer stability in aqueous buffers also may be compatible with buffers containing up to 15% (v/v) of these organic modifiers. The choice of buffer and buffer pH used for organic modifier screenings can incorporate the results of aqueous buffer stability studies. In one aspect, these modifiers are analyzed in buffers between pH 6.5 and pH 8.0, or, between pH 5.5 and pH 9.0, or between pH 4.5 and pH 10.0.

In one aspect, the chiral selectivity of chimeric cannulae monomer and/or polymers of the invention and the yield of the chiral selection methods of the invention are determined using capillary electrophoretic methods. In one aspect, the chiral selectivity method is evaluated using capillary electrophoretic methods and racemic mixtures of commercially available compositions, e.g., beta-blockers or equivalents. These methods also can be used to evaluate the efficiency (e.g., the chiral selectivity) of various embodiments of the invention, e.g., regular, helical nanotubules comprising chimeric and/or wild type CanA, CanB, CanC, CanD, CanE, etc. or mixed species polymers. Data obtained from stability studies also can be used to determine by routine screening optimal buffer pH, acceptable additives, and organic modifier concentrations, depending on the desired outcome of a particular chiral separation protocol.

In one aspect, the resolution obtained with polymers (e.g. nanotubular chimeric cannulae) and monomers of the invention is determined using commercially available chiral selectors. There are numerous published methods for separating racemic mixtures of racemic compositions, e.g., beta-blockers, using commercial chiral selectors with, e.g., capillary electrophoresis. These methods can utilize both protein and non-protein chiral selectors. In one aspect, tests incorporating commercially available enantio-separations media provide data about the comparative efficiency of nanotubular chimeric cannulae polymers and monomers of the invention as chiral selectors.

In one aspect, a chiral selectivity method of the invention or the resolution obtained with a polymer (e.g., nanotubular chimeric cannulae) and/or monomer of the invention is evaluated using capillary electrophoretic methods and racemic mixtures of commercially available beta-blockers, such as, e.g., those listed below:

| Compound | Structure |
|---|---|
| Sotalol | 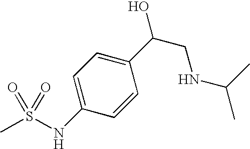 |
| Atenolol | 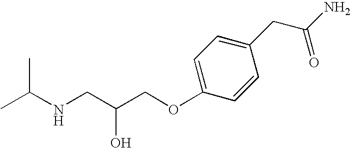 |
| Acebutolol | 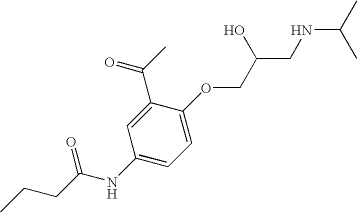 |
| Pindolol | 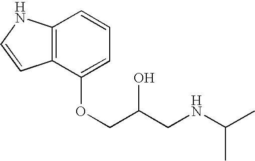 |
| Metoprolol | 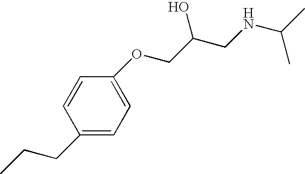 |
| Propranolol | 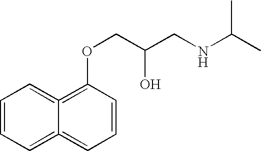 |
| Alprenolol | 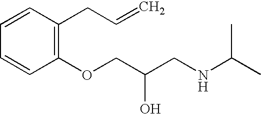 |
| Labetalol | 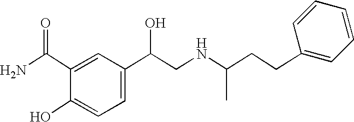 |

In one aspect, a chiral selectivity method of the invention or the resolution obtained with polymers and monomers of the invention is evaluated using capillary electrophoretic methods and racemic mixtures of propanolol. There are numerous reports in the literature that describe the resolution of enantiomers of propanolol, making it a good benchmark for the routine screening for optimizing chiral separations methods conditions employing the compositions of the invention, e.g., chimeric cannulae monomers and polymers (including nanotubules). Enantioseparation of propanolol has been accomplished using quail egg white riboflavin binding protein (see, e.g., De Lorenzi (1997) supra), pepsin, cellobiohydrolase, and bovine serum albumin (see, e.g., Tanaka (2001) J. of Biochem. Biophysical Methods 48:103-116; Henriksson (1996) FEBS Letters 390:339-344).

In one aspect, monomers of polymers of the invention are immobilized on a surface, e.g., a capillary. In one aspect, the methods of the invention are practice in a capillary tube, e.g., a GIGAMATRIX™ (Diversa Corporation, San Diego, Calif.). Both untreated and polyacrylamide-coated capillaries can be used to practice the methods of the invention. Untreated capillaries may be unsuitable for chiral selection due to adsorption of a chiral selector or an analyte on the walls of the capillary, see, e.g., Tanaka (2001) supra.

As discussed above, any separation fluid or organic modifier can be used to practice the methods of the invention. Determining optimal conditions by routine screening can be based on an optimization procedure described by Allenmark, S. G. Chromatographic Enantioseparation. Methods and applications. pg 90-141. 1998. West Sussex, England, Ellis Horwood Limited. This exemplary protocol uses a neutral buffer without additives or modifiers as the starting condition for separation. If the enantiomers are not resolved, the pH can be adjusted to pH 5.5 or 8.5. If one of these pH conditions results in loss of sample due to excessive complexation with a chimeric cannulae monomer or polymer of the invention, a low percentage of an organic modifier can be introduced. Changes also can be made to the buffer pH, choice of organic modifier, and concentration of organic modifier to improve resolution.

In one aspect, routine screening methods are carried out using a partial filling technique, as described, e.g., by Tanaka (2001) supra; Chankvetadze (2001) J. of Chromatography A. 906:309-363. In this exemplary technique the capillary (e.g., GIGAMATRIX™, Diversa Corporation, San Diego, Calif.) is only partially filled with the protein chiral selector (a chimeric cannulae monomer or polymer of the invention). This can minimize the sensitivity issues associated with the high UV backgrounds produced by protein at the detector. Using this method, it is possible to use up to 500 uM protein during the enantioseparation. A countercurrent technique can also be used. In countercurrent separations, conditions are used such that there is electrophoretic migration of the protein chiral selector (a chimeric cannulae monomer or polymer of the invention) away from the detector while the analyte migrates past the detector, see, e.g., Chankvetadze (2001) supra.

In alternative aspects, monomer or polymers or mixtures thereof are used to practice the methods of the invention. Chimeric cannulae monomers can have the ability to self-assemble into nanotubules. In one aspect, the chiral resolving power of different polymers (e.g., heteropolymers comprising chimeric and wild type cannulae proteins) and monomers relative to the resolving power of other polymers and monomers can be determined by routine screening, e.g., as described herein. By assembling into a nanotubule, a chimeric cannulae protein becomes a macromolecular structure that possesses distinct microenvironments, including an interior surface, cavity and an exterior surface. In addition, the regular assembly of the subunits into a helical structure introduces additional chirality into the polymer. The polymers of the invention include varying amounts of chirality, as varying amounts of chirality can enhance the enantioselectivity of the composition. The monomers and polymers of the invention can be designed to have varying constrained quaternary (4°) structures. In one aspect, varying constrained quaternary (4°) structures results in varying amounts of chiral selectivity.

In one aspect, the chiral selection is performed under cooling conditions and in the absence of sufficient divalent cation (less than 1 mM) so a cannulae monomer (e.g., a CanA monomer) will not self-assemble during chromatography.

In one aspect, the performance of the chiral selective compositions of the invention are compared to the performance of commercially available chiral selectors. In one aspect, beta-blocker resolutions are performed with capillaries packed with cellobiohydrolase or α₁-acid glycoprotein (ChromTech AB Cheshire, UK) using, e.g., the separation conditions provided by the supplier. Comparisons also can be made to separations obtained using highly sulfated cyclodextrans (Beckman Coulter, Fullerton, Calif.) according to, e.g., methods available from their applications guide. Other characteristics, such as good stability or minimal interference with analyte detection, can also be evaluated. Chimeric cannulae proteins of the invention, including the chimeric CanA polypeptide made by inserting peptide domains into a nonessential surface-exposed domain of CanA (see FIG. 1), can be evaluated using these routine screening methods. FIG. 1 is an illustration of a transmission electron micrograph of nanotubules assembled from recombinant CanA expressed in E. coli.

Because of the macromolecular similarity of nanotubular cannulae polymers to eukaryotic microtubules, any of the analytical methods that have been established in the microtubule field can be used to analyze chimeric cannulae polymers of the invention; see, e.g., Frederiksen, D. W. and L. W. Cunningham. Structural and Contractile Proteins, Part B: The Contractile Apparatus and the Cytoskeleton. 1982. Methods in Enzymology 85[Part B]. In evaluating the chiral selectivity of a chimeric cannulae monomer and/or polymer of the invention and the yield of the chiral selection methods of the invention, light and electron microscopy (e.g., transmission electron microscopes), differential centrifugation, size exclusion chromatography and/or turbidity measurement methods can be used. Each of these methods provides slightly different information about the stability and integrity of the assembled chimeric cannulae polymer.

The assembly and disassembly of polymers of the invention can be followed by measuring changes in solution turbidity, e.g., as described in Purich, D. L., et al. (1982) Microtubule disassembly: a quantitative kinetic approach for defining endwise linear depolymerization. Methods in Enzymology 85[Part B], 439-450. In one aspect, kinetic turbidity measurements are used. Kinetic turbidity measurements can be used to reflect changes in polymer weight concentration. These measurements can be used to determine rates of depolymerization. In one aspect, solution turbidity is monitored spectro-photometrically at 350 nm in a long path length cuvette. The long path length can provide an enhancement of the absorbance change improving sensitivity of the assay. In one aspect, the method comprises a long path length and a temperature-controlled cuvette containing buffers that can range in pH from 3 to 10. Stability of polymer can be measured by diluting concentrated solutions of polymer into the cuvette containing temperature-equilibrated buffer.

In one aspect, the chiral selectivity of chimeric cannulae monomer and/or polymers of the invention and the yield of the chiral selection methods of the invention are determined using a chiller-cooled system. The stability of polymer can be evaluated over a range of temperatures, e.g., from about 4° C. to 80° C. for each buffer pH. In one aspect, if it is not possible to measure accurate depolymerization rates using the rapid dilution method (due to over-dilution of the polymer into the test buffer), a resuspension method can be utilized. In the resuspension method, a wide-bore pipette can be used to resuspend polymer pellets in temperature-equilibrated buffer. The resuspended pellet then can be transferred to a cuvette for analysis. The advantage of this method is the ability to use more concentrated polymer solutions. The drawback, however, is variability introduced by potential shearing of the polymer during resuspension.

In one aspect, the chiral selectivity of chimeric cannulae monomer and/or polymers of the invention and the yield of the chiral selection methods of the invention are determined using differential centrifugation. Differential centrifugation can be used to assess the distribution of monomer protein incorporated into polymer vs. monomer free in solution. The differential centrifugation assay is useful for longer time course stability evaluations. In these assays, polymer that has been assembled under standard conditions at neutral pH can be pelleted by centrifugation and then resuspended in a buffer (e.g., at varied pH, such as from pH 3 to pH 10) and pre-equilibrated at a specified temperature (e.g., at varied temperature, such as a range from about 4° C. to 80° C.). The samples can be incubated at temperature for 2 to 24 hrs and then re-centrifuged to pellet the intact polymer. The supernatant and pellet fractions can be analyzed by SDS-PAGE. The supernatant will contain any soluble monomer (released by polymer depolymerization) and the pellet will contain intact polymer.

In one aspect, the chiral selectivity of chimeric cannulae monomer and/or polymers of the invention and the yield of the chiral selection methods of the invention are determined using size exclusion chromatography. Size exclusion chromatography can be used to analyze the overall size distribution of polymers. Polymer samples can be resuspended in buffer (e.g., at varied pH, such as from pH 3 to pH 10) and incubated for 24 hours at 4° C. Following incubation, the samples can be fractionated, e.g., on a Sephacryl S-1000 column (Amersham Pharmacia, Piscataway, N.J.). This size exclusion column will separate the micron length polymer from shorter polymers and oligomers. Because polymers can be extremely stable at 4° C. and neutral pH, and this buffer treatment can be used as the control.

In one aspect, the chiral selectivity of chimeric cannulae monomer and/or polymers of the invention and the yield of the chiral selection methods of the invention are determined using light microscopy, e.g., video-enhanced light microscopy, including both phase and differential interference contract (DIC) optics. Light microscopy can be used to evaluate the gross morphology of polymers following extended incubations (e.g., between about 24 to 48 hours) at varied pH, such as from pH 3 to pH 10. Light microscopy can provide useful information about the extent of nanotubule polymer bundling. It also can be used to detect the presence of larger protein aggregates.

In one aspect, the chiral selectivity of chimeric cannulae monomer and/or polymers of the invention and the yield of the chiral selection methods of the invention are determined using electron microscopy (EM), e.g., standard negative stain transmission electron microscopy. Electron microscopy can be used to look at the fine structure of nanotubules. EM can be useful for the analysis of periodicity and helicity of the intact polymers. In addition, EM can detect other protein assemblies that may form during incubation at various pH values or in the presence of organic modifiers. Depending on the incubation conditions, eukaryotic microtubules have been shown to assemble into a number of macromolecular structures including ring, sheets, and ribbons, as described, e.g., in Hyams, J. S, and C. W. Lloyd. Microtubules. 1993. New York, Wiley-Liss. The polymers of the invention can be modified to assemble or reassemble into such alternate structures.

Chimeric cannulae protein of the invention can be abundantly and economically expressed as a recombinant protein in vitro or in vivo using any expression system, including bacteria, yeast, mammalian or plant expression systems (e.g., as host cells), as discussed below.

In some aspects, the protein nanotubes of the invention have advantages over traditional carbon nanotubes (which were discovered by the Japanese microscopist Sumio lijima in 1991). These advantages can stem from the powerful capabilities afforded by a biological system over the carbon based system. In particular, the protein polymer of the invention is evolvable, allowing amino acid changes to be incorporated into the protein while preserving the polymer structure. In one aspect, the invention provides methods for modifying polypeptides of the invention, as described herein. This allows the protein to be modified in a way that changes its chemical characteristics, such as charge and hydrophobicity, thereby greatly expanding the number of applications for the polymer. For example, compositions of the invention can comprise circuits and transistors.

With the ability of the biological nanotube proteins to bind to GFP proteins, nanotubes of the invention can be used as fluorescent tubes. In one aspect, this can greatly increase the accuracy of data received through microscopic viewing. Due to the ability of the biological nanotubes to modify their chemical makeup, there are endless medical applications for use of nanotubes of the invention. Presently, patients are frequently rejecting transplants, and transfusions. However, in some aspects, with the use of biological nanotubes of the invention, the make-up of the proteins might be altered, and even be made into working organs and eliminating some problems with organ and blood rejection. On an electrical scale, due to the biological nanotubes' ability to change chemical make-up, the charge of nanotubes of the invention can also be manipulated to become an excellent composition for devices for current conduction, e.g., conducting wires, plates, transistors, and the like. Thus, nanotubes of the invention can be made into computer transistors, e.g., supercomputers, but at a miniscule size, e.g., in some aspects, only a few nanometers in length. In one aspect, the nanotubes of the invention comprises copper-comprising proteins, iron-comprising proteins, or proteins comprising other metal-conductive ions. Alternatively, a conductive substance, e.g., a metal ion, can be fused or attached to a monomer or nanotubules of the invention in any manner, e.g., by charge to the heterologous polypeptide or peptide of a chimeric protein of the invention.

Unlike carbon nanotubes, biological nanotubes of the invention can be manufactured in microbes or in plants; in some aspects, at an exceptionally low cost. While carbon nanotubes are manufactured by use of natural gases such as methane, the only necessary energy needed in manufacturing carbon nanotubes would be the light of the sun and water, used in plants. With this free source of energy, and no harmful byproducts in its production of these natural polymers, all that remains are the benefits of these biological nanotubes. Presently, carbon nanotubes are produced, with yield as low as one to two pounds a day, costing up to nine hundred dollars a gram. With this incredibly high cost of manufacturing, it is currently unrealistic to expect research in the carbon nanotubes to be useful for all applications. In contrast, in some aspects, proteins of the invention can be generated at an exceedingly lower price, and with many applications.

Another important aspect of biological nanotubes of the invention is that unlike carbon nanotubes, the proteins of the invention can be evolved into continuously improved types of protein nanotubes for almost any need. Through the use of biological mutations, the protein can be manipulated to become a universal enzyme, capable of manipulating itself into any substrate. In one aspect, it can be used for advanced camouflage in the military, e.g., in one aspect, it is able to change its color in any surrounding to match identically to the environment. Carbon nanotubes, though very strong, are rigid and do not form the same flexible shapes that proteins can. In some aspects, protein nanotubes of the invention can be formed into any shape, can withstand temperatures up to 150° C., can change its chemistry to be a universal donor, and can be evolved into new proteins.

In some aspects, there are a differences between carbon nanotubes and biological nanotubes of the invention. In one aspect, the first difference is that a nanotube of the invention can be generated from a living organism (note: in an alternative aspect, a process of the invention can generate a nanotubules in a cell-free or synthetic system, e.g., in vitro). Secondly, the bonds are different in each case. In the carbon nanotubes, the carbon atoms are held tightly together by strong covalent bonds. Carbon nanotubes, also known as “bucky-balls” are composed of C60, sixty covalently bonded carbon atoms. Covalent bonds occur when atoms share electrons, either in “free-loader” bonds, single bonds, double bonds, or triple bonds. Covalent bonds are the strongest type of molecular bonds, having high boiling points, but are also very rigid. Since carbon nanotubes contain rigid covalent bonds, they do serve as excellent materials for buildings, however, for more practical uses, their rigidness inhibits them from being used easily in everyday products, such as clothing and fibers, and other textiles.

However, in the biological protein nanotubes of the invention the proteins are held together by peptide bonds. These peptide covalent bonds are not as strong as the covalent bonds holding together the carbon in the carbon nanotubes. This may cause a lower heat resistance. However, with a heat resistance of up to approximately 150° C., protein nanotubes of the invention can still be used for any heat-resistant application, e.g., in earthquake resistant building materials and other such applications. For example, polypeptide polymers of the invention can be used as flame retardants and can be incorporated into any material, e.g., fabrics (which can be designated NANOAVID™ textiles), fibers, adhesives and the like. In one aspect, polypeptide polymers of the invention are used to make a material more heat resistant. Also, with the peptide bonds, the tubules of the invention are more flexible, allowing them to be used in textiles and fibers, e.g., clothing or other such products, without the restrictions of the carbon nanotubes. Thus, the nanotubules and processes of the invention can be used in any aspect of nanotechnology.

### Cannulae Polypeptides and Peptides

The invention provides chimeric polypeptides that can be used as vaccines, immunomodulatory compositions, biosynthetic pathways, as chiral selectors and enantiomeric selection devices, in medical devices, prosthetics, nanostructures or nanomachines and the like. In one aspect, the invention provides polypeptides comprising at least a first domain comprising a cannulae polypeptide and at least a second domain comprising a heterologous polypeptide or peptide, carbohydrate, small molecule, nucleic acid or lipid. The chimeric (fusion) cannulae polypeptides of the invention can be recombinant proteins encoded by nucleic acids comprising fusion of the sequence of a cannulae monomer to other protein or peptide coding sequences (heterologous sequences) to produce cannulae fusion (chimeric) proteins. However, the chimeric (fusion) cannulae polypeptides of the invention can be joined to the heterologous polypeptide or peptide, carbohydrate, small molecule, nucleic acid or lipid by any means, including linkers. The chimeric (fusion) cannulae polypeptides of the invention can be partly or entirely synthetic. In one aspect, the chimeric monomers of the invention can form dimers, trimers (polymers of any length) and/or they can assemble, e.g., self-assemble, into a higher order structure, e.g., a quaternary structure, such as a nanotubule. The heterologous sequences can be added to the cannulae protein's amino- or carboxy-terminal end, or, they can be added internal to the cannulae protein.

In one aspect, a subsequence of a chimeric (fusion) cannulae polypeptide of the invention is removed. In one aspect, a subsequence of a chimeric (fusion) cannulae polypeptide of the invention is removed and replaced by a heterologous polypeptide or peptide. Alternatively, the heterologous polypeptide or peptide can be added to another section of the monomer (i.e., distal to the removed subsequence). The removed subsequence can be amino- or carboxy-terminal, or, it can be internal to the cannulae protein. In one aspect, the subsequence of fusion (hybrid) CanA protein that is removed and replaced by a heterologous polypeptide or peptide is a 14 residue motif consisting of residue 123 to residue 136 of SEQ ID NO:2 (i.e., “PDKTGYTNTSIWVP”), or, a 17 residue motif located at amino acid residue 123 to residue 139 of SEQ ID NO:2, (i.e., “PDKTGYTNTSIWVPGEP”). When the CanA monomer is in polymeric nanotubular form, a 14 residue motif consisting of residue 123 to residue 136 of SEQ ID NO:2 or a 17 residue motif located at amino acid residue 123 to residue 139 of SEQ ID NO:2 is expressed on the outer surface of the nanotubule. In one aspect, the tubule can then act as a high-density chiral selector. The surface-exposed 14 or 17 amino acid domain in CanA is not essential for self-assembly of nanotubules. Thus, these domains can serve as a site for the insertion of peptides, e.g., with chiral selector properties, ligand binding properties, and the like.

In one aspect, the polypeptides of the invention comprise truncated versions of cannulae proteins. For example, in alternative aspects, a cannulae protein in a monomer or polymer of the invention comprises a truncation of sequences equivalent to a complete or partial removal of signal sequences, e.g., the first 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30, or more, amino terminal amino acid residues. For example, in CanA, CanB or CanC-comprising compositions of the invention, in one aspect the signal sequence is removed (e.g., the first 25 amino acids is removed); and, in one aspect, a “start” Met (methionine) is subsequently added. Carboxy terminal, or internal, residues can also be removed, and, in some aspects, replaced by heterologous residues.

The invention provides polymers comprising a chimeric protein of the invention, a cannulae protein, or a mixture thereof. Once assembled, e.g., as a nanotubule, bundle, ball, sheet, fiber, filament, thread and the like, chimeric cannulae proteins of the invention can serve as a molecular scaffold that displays its heterologous sequence (its chimeric/fusion protein partner) in a defined orientation, e.g., in a regular, helical array on a tubule, nanotube, bundle, ball, filament or thread. This functional flexibility offers the opportunity to display a large variety of recombinant proteins on the surface of a nanotubule to create chiral selectors with a wide range of applications. The heterologous sequences can be chiral selection motifs, enzymes, active sites, epitopes, ligands, receptors, antigens, antibodies or antigen binding sites, nucleic acid binding proteins, a cell matrix binding motif, carbohydrate binding motifs, and the like.

In one aspect, the chimeric cannulae monomers are overexpressed in a host cell, e.g., a bacteria such as an E. coli. In one aspect, the overexpressed polypeptide is modified by nucleic acid mutagenesis and/or directed protein evolution, as described herein.

The cannulae domain of the chimeric polypeptides of the invention can comprise a CanA polypeptide as set forth in SEQ ID NO:2 (encoded, e.g., by SEQ ID NO:1); a CanB polypeptide as set forth in SEQ ID NO:4 (encoded, e.g., by SEQ ID NO:3); a CanC polypeptide as set forth in SEQ ID NO:6 (encoded, e.g., by SEQ ID NO:5); a CanD polypeptide as set forth in SEQ ID NO:8 (encoded, e.g., by SEQ ID NO:7); a CanE polypeptide as set forth in SEQ ID NO:10 (encoded, e.g., by SEQ ID NO:9), or the consensus cannulae protein SEQ ID NO:12 (encoded, e.g., by SEQ ID NO:11). The cannulae domain of the chimeric polypeptides of the invention also can comprise a polypeptide having a 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to polypeptide as set forth in SEQ ID NO:2, SEQ ID NO:4, SEQ ID NO:6, SEQ ID NO:8, SEQ ID NO:10 or SEQ ID NO:12, wherein the cannulae domain polypeptide can form a nanotubule and/or can act as a chiral selector (in monomeric or polymeric form). The cannulae domain of the chimeric polypeptides of the invention also can comprise a polypeptide encoded by a nucleic acid having a 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a nucleic acid as set forth in SEQ ID NO:1, SEQ ID NO:3, SEQ ID NO:5, SEQ ID NO:7, SEQ ID NO:9 or SEQ ID NO:11, wherein the cannulae domain polypeptide can form a nanotubule and/or can act as a chiral selector (in monomeric or polymeric form). The cannulae domains of the chimeric polypeptides of the invention can comprise two or more of these proteins, including mixtures of CanA, CanB, CanC, CanD and/or CanE and/or the cannulae protein representing the consensus sequence (SEQ ID NO:12).

In another aspect, a cannulae domain of a chimeric polypeptide of the invention, or a polymer of the invention, in addition to one or more of CanA, CanB, CanC, CanD and/or CanE and/or the cannulae protein representing the consensus sequence (SEQ ID NO:12), including mixtures thereof, and related proteins (e.g., having at least 50% to 100% sequence identity to an exemplary cannulae protein, e.g., as described herein) can also comprise a FtsZ protein, or a protein or peptide comprising a self-assembling fragment thereof (a FtsZ domain). FtsZ is another self-assembling polymer protein that can be used in the compositions (e.g., the chimeric protein monomers, or polymers) or methods of the invention. FtsZ is well-described in the art, see, e.g., Yan (2001) “Regions of FtsZ important for self-interaction in Staphylococcus aureus,” Biochem Biophys Res Commun. 284(2):515-518; Rivas (2001) “Direct observation of the enhancement of noncooperative protein self-assembly by macromolecular crowding: indefinite linear self-association of bacterial cell division protein FtsZ,” Proc. Natl. Acad. Sci. USA 98(6):3150-3155; Rivas (2000) “Magnesium-induced linear self-association of the FtsZ bacterial cell division protein monomer. The primary steps for FtsZ assembly,” J Biol Chem. 275(16):11740-9; Sossong (1999) “Self-activation of guanosine triphosphatase activity by oligomerization of the bacterial cell division protein FtsZ,” Biochemistry 38(45):14843-148450.

In one aspect, at least one cannulae-encoding sequence (a nucleic acid encoding CanA, CanB, CanC, CanD and/or CanE and/or the cannulae protein representing the consensus sequence (SEQ ID NO:12), including mixtures thereof, and related proteins, e.g., having at least 50% to 100% sequence identity to an exemplary cannulae protein SEQ ID NO:2, SEQ ID NO:4, SEQ ID NO:6, SEQ ID NO:8, SEQ ID NO:10, SEQ ID NO:12) is fused with at least one FtsZ coding sequence (e.g., gene). In one aspect, incorporation of one or more FtsZ protein domains results in a bottle-brush like structure or a scaffold of some sort. The FtsZ-comprising polymers of the invention can be used for biocatalysis, separations, chromatography resin, antigen presentation, etc., including any use for a cannulae polymer of the invention, as described herein. In one aspect, the bottle-brush shaped polymer of the invention is “gel-like”; thus, the size of the “holes” between the “bristles” can be engineered and the polymer used, e.g., for sieving, biocatalysis, separations, chromatography and the like.

The term protein or polypeptide sequence or amino acid sequence includes an oligopeptide, peptide, polypeptide, or protein sequence, or to a fragment, portion, or subunit of any of these, and to naturally occurring or synthetic molecules. The terms “polypeptide” and “protein” include amino acids joined to each other by peptide bonds or modified peptide bonds, i.e., peptide isosteres, and may contain modified amino acids other than the 20 gene-encoded amino acids. The term “polypeptide” also includes peptides and polypeptide fragments, motifs and the like. The term also includes glycosylated polypeptides. The peptides and polypeptides of the invention also include all “mimetic” and “peptidoinimetic” forms.

The invention also comprises “variants” of the chimeric polynucleotides or polypeptides of the invention, and methods of making them, wherein the variants are modified at one or more base pairs, codons, introns, exons, or amino acid residues (respectively) yet retain the activity or have a modified activity of a chimeric polypeptide of the invention. Variants can be produced by any number of means included methods such as, for example, error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, GSSM™ and any combination thereof. Techniques for producing variant chimeric polypeptides having activity at a pH or temperature, for example, that is different from a template chimeric polypeptide, are included herein. The term “saturation mutagenesis” or “GSSM™” includes a method that uses degenerate oligonucleotide primers to introduce point mutations into a polynucleotide, as described in detail, below. The term “optimized directed evolution system” or “optimized directed evolution” includes a method for reassembling fragments of related nucleic acid sequences, e.g., related genes, and explained in detail, below. The term “synthetic ligation reassembly” or “SLR” includes a method of ligating oligonucleotide fragments in a non-stochastic fashion, and explained in detail, below.

In one aspect, nucleic acids encoding the chimeric polypeptides of the invention are cloned and over-expressed in a host cell, e.g., bacterial (e.g., E. coli, Bacillus, Streptomyces), yeast, plant or mammalian host cells.

Purified recombinant chimeric cannulae protein of the invention can self-assemble into nanotubules. In some aspects, presence of a divalent cation may be needed, depending on the conditions and mixture of polypeptides comprising the nanotubular assembly or the presence of proteins that catalyze or facilitate tubule assembly. Thus, in one aspect the chimeric cannulae proteins of the invention or the nanotubules of the invention are assembled in the presence of a divalent cation. The divalent cation may be Ca²⁺, Mg²⁺, Cu²⁺, Zn²⁺, Sr²⁺, Ni²⁺, Mn²⁺ and/or Fe²⁺. In one aspect, a single divalent cation is needed, e.g., Ca²⁺ or Mg⁺. In another aspect, both Ca²⁺ and Mg²⁺ are needed for chimeric cannulae protein can self-assemble into nanotubules. In one aspect, the divalent cation(s) are present in millimolar concentrations.

In alternative aspects, chimeric proteins of the invention or nanotubules of the invention are assembled in the presence of one or more initiators, which can be one or more environmental conditions, e.g., increased temperature, pH or salinity, and/or one or more compositions as an initiator, e.g., a partially polymerized monomer as a primer or any element found in the original environment of the Pyrodictium abyssi organism, from which the canA, canB, canC, canD and canE genes were initially derived. For example, in one aspect, the chimeric proteins of the invention or nanotubules of the invention are assembled in the presence of seawater, or equivalent, from the growth microenvironment of the Pyrodictium abyssi organism, or equivalent organisms which form CanA-like nanotubules. For example, in one aspect, the chimeric proteins of the invention or nanotubules of the invention are assembled in the presence of black-smoker fluid, or equivalent. Equivalent environments that can be used in the methods of the invention for the assembly of chimeric proteins of the invention or nanotubules of the invention include fluids comprising the same, substantially the same, or a selected subset of elements found in the growth microenvironment, e.g., black-smoker fluid, or equivalent. For example, the methods of the invention (e.g., for polymerizing chimeric polypeptides of the invention, or to assemble nanotubules of the invention) can comprise use of any mixture of salts, e.g., iron sulfate, manganese sulfate, lead sulfate, lithium sulfate, manganese chloride and/or calcium chloride or equivalent salts. The invention provides methods for the controlled polymerization of proteins of the invention in the presence of different catalyst salts, such as iron sulfate, manganese sulfate, lead sulfate, lithium sulfate, manganese chloride and/or calcium chloride or equivalent salts. In one aspect, the polymerization takes place in a solution. In one aspect, the controlled polymerization conditions can further comprise modification of temperature, salinity, pH and the like. The methods of the invention also can comprise use of some, or all elements described in Table I (e.g., H₂S, H₂, CH₄, Mn, Fe, Be, Zn, Cu, Ag, Pb, Co, Si, Al, Ba, Cs, Li, Rb, CO₂, Ca, Sr, B, As Se, P, Mg, SO₄, and/or Alk), wherein the concentrations of elements set forth in Table 1 are only alternative embodiments to practice the assembly processes of the invention. In one aspect, copper sulfate salt is used as an initiation inhibitor or depolymerization element, particularly when used as an isolated element, versus one of many elements in a complex growth environment solution comprising many salts and elements.

The chimeric polypeptide of the invention can comprise the cannulae polypeptides CanA, CanB, CanC, CanD and/or CanE, and subsequences and mixtures thereof. In the following alignment, CanA and CanA_pep stand for nucleic acid SEQ ID NO:1 and its corresponding amino acid SEQ ID NO:2, respectively; CanB and CanB_pep stand for nucleic acid SEQ ID NO:3 and its corresponding amino acid SEQ ID NO:4, respectively; CanC and CanC_pep stand for nucleic acid SEQ ID NO:5 and its corresponding amino acid SEQ ID NO:6, respectively; CanD_partial stands for nucleic acid SEQ ID NO:7 or its corresponding amino acid SEQ ID NO:8; and CanE_partial stands for nucleic acid SEQ ID NO:9 or its corresponding amino acid SEQ ID NO:10.

Nucleic acid alignment for SEQ ID NOS: 1, 3, 5, 7, and 9:

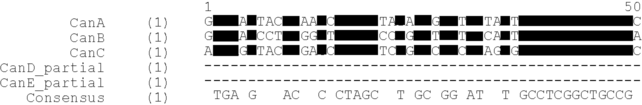

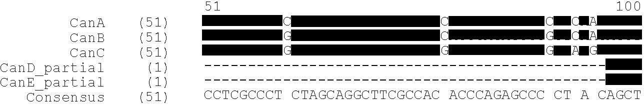

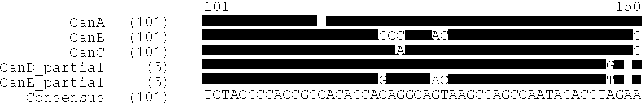

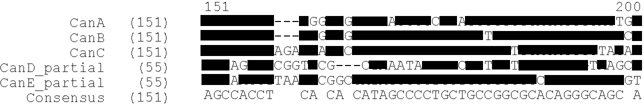

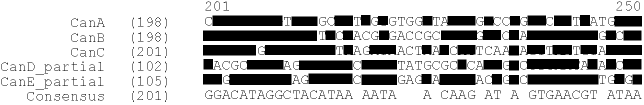

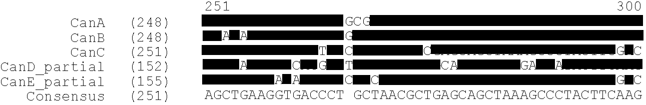

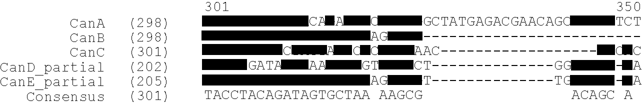

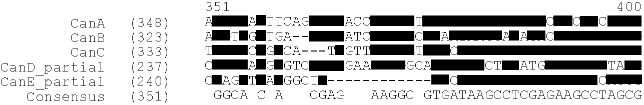

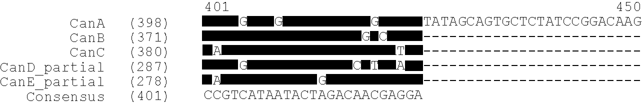

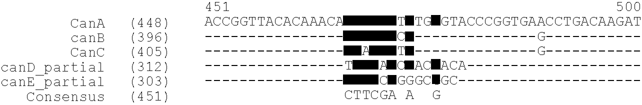

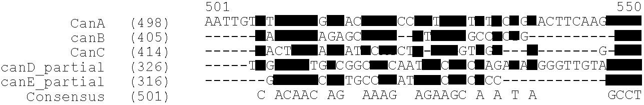

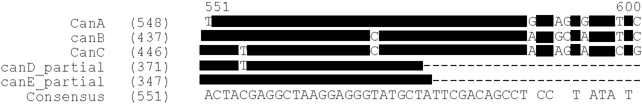

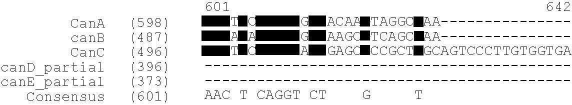

Amino Acid Alignment for SEQ ID NOS: 2, 4, 6, 8, and 10:

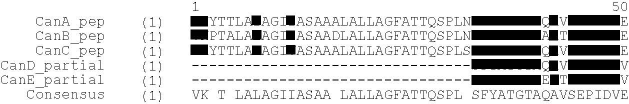

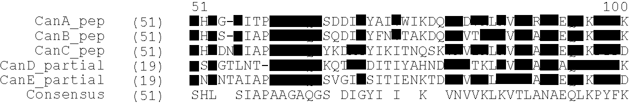

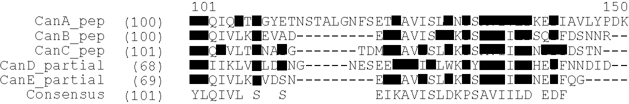

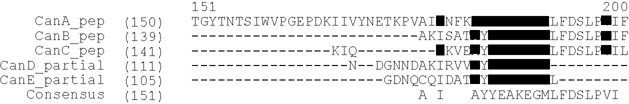

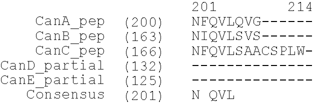

A polymer of the invention may have a shape of a short fiber, and therefore is also called “polymer fiber.” A polymer fiber of the invention comprise monomeric protein units, e.g. CanA: 182 amino acids: MW=19,830 Daltons, having a sequence of SEQ ID NO:2. The secondary structure of a protein of the invention can be mainly β-sheets.

The protein subunits in a polymer of the invention can be arranged in a right-handed or left-handed, two-stranded helix. In alternative aspects, polymer fibers of the invention are made up of a three-handed helix. In one aspect, the periodicity (the distance of one helix turn to the next) of the polymer is 4.4 nm. In one aspect, a polymer of the invention has a unique quaternary structure. In one aspect, a polymer fiber of the invention has an outer diameter of 25 nm and inner diameter, 21 nm (in suspension). Under an electronic microscope, the dry negatively stained polymer fibers exhibit an outer diameter of 32 nm due to collapsing. Length of the polymer fiber of the invention can be between 3 and 5 micrometers. Some of the polymer fibers of the invention may reach a length from 10 to 25 micrometers. The polymer fibers of the invention may form bundles of tens and hundreds of fibers with an overall diameter of 100 to 500 nm. In one aspect, the bundle may reach an overall diameter of 4,000 nm. In one aspect, the polymer fiber is at least stable up to 128° C., or more.

Cannulae polypeptides of the invention, e.g., CanA-, CanB-, CanC-, CanD-, and/or CanE-comprising polypeptides of the invention, including the consensus sequence, SEQ ID NO:12, including fibers comprising polypeptides of the invention, or textiles comprising polypeptides of the invention, are heat-resistant protein that can form nanotubules. Cannulae polypeptides of the invention, e.g., CanA-, CanB-, CanC-, CanD-, and/or CanE-comprising polypeptides of the invention, including the consensus sequence, SEQ ID NO:12, can assemble from monomeric subunits that self-assemble in the presence of divalent cation, e.g., in one aspect, into hollow rods, e.g., with an outer diameter of approximately 25 nm and an inner diameter of approximately 20 nm. Cannulae polypeptide-comprising (CanA-, CanB-, CanC-, CanD-, and/or CanE-comprising-) monomers of the invention can be heat-stable. CanA-comprising monomers of the invention can be rapidly purified from bacterial extracts following heat treatment to remove the majority of the heat-labile host proteins. Following purification, the CanA-, CanB-, CanC-, CanD-, and/or CanE-comprising monomers of the invention can self-assemble into nanotubules in the presence of the appropriate cation, e.g. calcium and magnesium, at elevated temperature. The assembled nanotubule structures contain CanA-, CanB-, CanC-, CanD-, and/or CanE-comprising monomers of the invention arranged with a helical pitch. Cannulae polypeptides of the invention, e.g., CanA-, CanB-, CanC-, CanD-, and/or CanE-comprising polypeptides of the invention, including the consensus sequence, SEQ ID NO:12, can be heat stable, e.g., up to 128° C., or more, and, in one aspect, can remain assembled in the presence of SDS or high concentrations of urea.

Cannulae polypeptide-comprising (CanA-, CanB-, CanC-, CanD-, and/or CanE-comprising-) nanotubules of the invention can exhibit remarkable heat stability, e.g. temperatures up to about 150° C. or 140° C. In one aspect, the nanotubules of the invention have heat stability in temperatures up to 128° C. and stability in 2% SDS at 100° C. for at least 60 minutes. Purified recombinant CanB protein will also form nanotubular structures but they are less regular and not as heat stable as the nanotubules assembled from CanA. Together, CanA (SEQ ID NO:2), CanB (SEQ ID NO:4), and CanC (SEQ ID NO:6) represent three very similar proteins that exhibit significantly different polymerization potentials in vitro, as summarized in Table 2:

TABLE 2
Comparison of amino acid sequences of CanA, CanB, CanC.

| Protein | CanA | CanB | CanC |
|---|---|---|---|
| CanA | 100% | | |
| CanB | 60% Identical | 100% | |
| | 64% Similar | | |
| CanC | 55% Identical | 68% Identical | 100% |
| | 62% Similar | 77% Similar | |

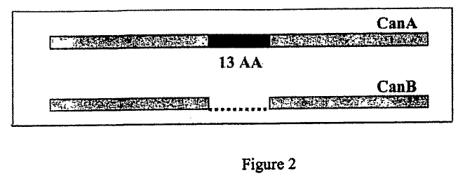

One difference between CanA and CanB is the 14 amino acid insertion near the middle of the CanA sequence (see FIG. 2). Immunoelectron microscopy and an antibody specific for this 14 amino acid sequence have been used to determine that this sequence is displayed on the surface of the assembled nanotubule. The absence of this corresponding sequence in CanB demonstrates that this peptide domain is nonessential for nanotubule assembly. Therefore, it is possible to remove this sequence and replace it with a peptide domain that alters the structure of CanA. In one aspect, replacing the endogenous 14 residue motif with a heterologous peptide changes the enantioselectivity of CanA.

Recombinant chimeric proteins of the invention can be expressed in a cell, e.g., a bacteria, such as E. coli, and purified away from host proteins by using heat treatment to denature and precipitate (e.g., E. coli) protein. The soluble heat stable protein (e.g., CanA) can be recovered from the supernatant following centrifugation. The chimeric protein can be assembly-competent at this stage. In one aspect, the self-assembly reaction is initiated by addition of millimolar concentrations of Ca⁺⁺ and Mg⁺⁺. In one aspect, following assembly of the nanotubules, they are stable in cation-free buffer and buffers containing up to 20 mM chelator, e.g., EDTA, EGTA.

Colloidal Stability. Nanotubules of the invention can interact at different levels by pairing, bundling, entangling (excluded volume interaction) and electrostatic cross-linking (bridging by divalent cations). The different types of aggregates have an increasing dimensionality from a pair of rods to an interconnected network. The bundling of CanA nanotubules appears to be a magnesium-dependent process. In the absence of magnesium, CanA displays minimal bundling. However, upon the addition of millimolar concentrations of magnesium, CanA nanotubules will form bundles visible by standard phase contrast light microscopy.

Nanotubule Stiffness. CanA nanotubules have been imaged under the transmission electron (TEM) and atomic force microscopes (AFM). From analyses of thermal vibrations of a single fiber in vacuum under the TEM, it was found that the CanA bending modulus is about 5±2 GPa. This result is somewhat greater than other rigid biopolymers of the same dimensions, such as microtubules which have a bending modulus of nearly 2 GPa, and comparable to the bending moduli of the strongest synthetic polymer fibers like Poly(6-amide) or Poly(methylmethacrylate), or, PLEXIGLAS™).

In one aspect, the chimeric CanA, CanB, CanC, CanD, and/or CanE proteins of the invention are used as chiral selectors, e.g., in capillary electrophoresis. Serum albumin was one of the first proteins used as a chiral stationary phase for the successful separation of enantiomers, see, e.g., (Allenmark, 1998). Numerous proteins have been used to accomplish many enantioseparations using capillary electrophoresis methods. These proteins include α₁-acid glycoprotein, avidin, ovomucoid, transferrin, cytochrome c, lysozyme, pepsin, cellulase, and cellobiohydrolase see, e.g., Tanada (2001) supra. Proteins are favorable for use as chiral selectors because they frequently can be used for a wide variety of enantioseparations, see, e.g., Lloyd (1995) J. of Chromatography A. 694:285-296. In addition, because proteins can be used for chiral separations in aqueous buffers, they are a good choice for the analysis of samples derived from biological material, see, e.g., Busch (1993) supra. Accordingly, in alternative aspects, the chimeric CanA, CanB, CanC, CanD, and/or CanE polypeptides of the invention comprise chiral selection motifs from serum albumin, α₁-acid glycoprotein, avidin, ovomucoid, transferrin, cytochrome c, lysozyme, pepsin, cellulase and cellobiohydrolase. The chimeric CanA, CanB, CanC, CanD, and/or CanE of the invention can comprise any peptide motif having a chiral selection capability. These motifs can be inserted into a CanA, CanB, CanC, CanD, and/or CanE or added to a CanA, CanB, CanC, CanD, and/or CanE. In one aspect, they are used to replace a subsequence of CanA that has been removed, e.g., a 14 residue motif consisting of residue 123 to residue 136 of SEQ ID NO:2 or a 17 residue motif located at amino acid residue 123 to residue 139 of SEQ ID NO:2.

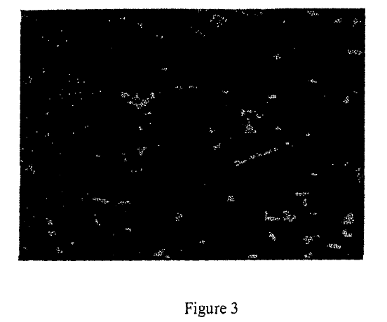

A chimeric monomer or polymer of the invention can comprise a detectable moiety. In one aspect, the heterologous motif is a detectable moiety, e.g., a green fluorescent protein. In one aspect, the invention provides a nanotubule comprising chimeric monomers comprising green fluorescent protein motifs. These monomers and nanotubules can be used to study nanotubule formation, dissolution and function. For example, FIG. 3 is an illustration of an immunofluorescent light microscope image of nanotubules assembled from a fusion protein generated by fusing the CanA open reading frame (SEQ ID NO:1) to the open reading frame of the green fluorescent protein ZSGREEN™ (BD Biosciences Clontech, Palo Alto, Calif.).

The invention provides enantioseparation methods using proteins free in solution as buffer additives, as described, e.g., in Busch (1993) supra, and using proteins immobilized by a variety of methods, as described, e.g., in Tanaka (2001) supra; Ito (2001) J. of Chromatography A 925:41-47. There are advantages and disadvantages to both approaches. By using proteins in solution, the native conformation of the protein is maintained resulting in a more uniform presentation of the sites involved in generating chiral resolution. However, in capillary electrophoresis-based methods, the presence of protein in the buffer solutions can produce extremely high background UV absorption. This limitation has been addressed by using partial filling and countercurrent techniques that allow relatively high concentration protein solutions to be used without causing background problems at the detector. Partial filling and countercurrent techniques are well known in the art, as, e.g., described in Tanaka (2001) supra; Chankvetadze (2001) supra.

In contrast, the use of immobilization techniques allows for the production of capillaries with high concentrations of the protein chiral selector. The potential drawback to these approaches is the heterogeneity introduced by the method of protein immobilization. This heterogeneity is particularly important when analyzing protein-ligand interactions (see, e.g., Lloyd (1995) supra). Changes in protein conformation introduced as a result of the immobilization method can significantly alter protein-ligand interactions and these types of analyses are therefore more often performed using protein chiral selectors in free solution.

Given their capacity for stereospecific molecular recognition (see, e.g., Lakshmi (1997) Nature 388:758-760; Henriksson (1996) supra), enzymes and apoenzymes are a source of chiral selectors used in the compositions and methods of the invention. Thus, the invention provides chimeric monomers and polymers, including nanotubules, comprising chiral selector enzymes and apoenzymes and chiral selector peptide motifs of enzymes and apoenzymes, such as enzyme active site motifs. The chimeric monomers and polymers, including nanotubules, of the invention can comprise any enzymes or apoenzymes, or any enzyme active site motif. For example, the chimeric monomers and polymers, including nanotubules, and active site motifs of the invention can be derived from glycosyltransferases, glycosylhydrolases, nitrilases, esterases, amidases, lipases, polymerases, cellulases, hydrolases, deaminases, nitroreductases and the like.

Polypeptides and peptide for making and/or using the chimeric monomers and polymers of the invention can be isolated from natural sources, be synthetic, or be recombinantly generated polypeptides. Peptides and proteins can be recombinantly expressed in vitro or in vivo. The peptides and polypeptides of the invention can be made and isolated using any method known in the art. Polypeptide and peptides for making and/or using the chimeric monomers and polymers of the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269:202; Merrifield (1997) Methods Enzymol. 289:3-13) and automated synthesis may be achieved, e.g., using the ABI 431A Peptide Synthesizer (Perkin Elmer) in accordance with the instructions provided by the manufacturer.

The peptides and polypeptides for making and/or using the chimeric monomers and polymers of the invention can also be glycosylated. The glycosylation can be added post-translationally either chemically or by cellular biosynthetic mechanisms, wherein the later incorporates the use of known glycosylation motifs, which can be native to the sequence or can be added as a peptide (e.g., a glycosylation motif) or added in the nucleic acid coding sequence (e.g., added as a glycosylation motif). The glycosylation can be O-linked or N-linked.

In one aspect, a glycosylated monomer and/or polymer of the invention is used in carbohydrate-based therapeutics, including inhibition of carbohydrate-lectin interactions; immunization, using monoclonal antibodies for carbohydrate antigens; inhibition of enzymes that synthesize disease-associated carbohydrates; replacement of carbohydrate-processing enzymes; targeting of drugs to specific disease cells via carbohydrate-lectin interactions; carbohydrate based anti-thrombotic agents. In one aspect, monomers and/or polymers of the invention present carbohydrates in a multivalent manner; carbohydrate-based therapeutics can be more effective when carbohydrates are presented in a multivalent manner. In one aspect, the assembled monomers of the invention (e.g., the polymers of the invention, or “pyrotex/nanodex nanotubles”) comprise repeating monomeric subunits each displaying a therapeutic carbohydrate. In one aspect, the carbohydrates are expressed on the inner or outer (or both) surface of a nanotubule of the invention. In this aspect, the assembled polymer serves as an ideal vehicle for the presentation of a multivalent carbohydrate therapeutic (in alternative aspects of the invention other biological agents, therapeutics or drugs, e.g., small molecules, proteins, peptides, nucleic acids, lipids, etc. can also be displayed in a like manner). The addition of the carbohydrate of interest could be accomplished by expressing a cannulae protein (e.g., CanA, B, C, D, E) monomer in a glycosylating host (e.g., a bacterial, fungal, or mammalian host) such that the host's glycosylation system adds a desired carbohydrate to the monomeric protein during heterologous expression. In one aspect, one or several (additional) glycosylation sites (e.g., N-linked sites or O-linked sites) are engineered into monomeric cannulae protein (e.g., CanA, B, C, D, E) coding sequence in a targeted position to create, or increase, the number of glycosylation sites present in a monomeric protein amino acid sequence. If a host cell does not express the monomer protein with the desired carbohydrate chain or if the glycosylation is non-uniform, in one aspect, the expressed monomers are processed in vitro with glycosidases and glycosyltransferases to first cut back the glycosylation added by the host expression system (glycosidases) and then re-build the carbohydrate chain using a series of glycosyltransferases to produce a monomer or assembled nanotubule polymer that uniformly displays the carbohydrate of interest. In one aspect, monomers displaying different carbohydrates of interest are produced and then assembled into a heteropolymer that displays multiple carbohydrate chains.

The peptides and polypeptides for making and/or using the chimeric monomers and polymers of the invention, as defined above, include all “mimetic” and “peptidomimetic” forms. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic is within the scope of the invention, i.e., that its structure and/or function is not substantially altered. Thus, in one aspect, a mimetic composition is within the scope of the invention if it has an amylase activity.

Polypeptide mimetic compositions can contain any combination of non-natural structural components. In alternative aspect, mimetic compositions of the invention include one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide of the invention can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

A polypeptide can also be characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1,-2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; D- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholinyl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions. Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues. Arginine residue mimetics can be generated by reacting arginyl with, e.g., one or more conventional reagents, including, e.g., phenylglyoxal, 2,3-butanedione, 1,2-cyclo-hexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitro-benzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4-hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A residue, e.g., an amino acid, of a polypeptide for making and/or using the chimeric monomers and polymers of the invention can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but also can be referred to as the R- or S-form.

The invention also provides methods for modifying the chimeric polypeptides of the invention by either natural processes, such as post-translational processing (e.g., phosphorylation, acylation, etc), or by chemical modification techniques, and the resulting modified polypeptides. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also a given polypeptide may have many types of modifications. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation. See, e.g., Creighton, T. E., Proteins—Structure and Molecular Properties 2nd Ed., W.H. Freeman and Company, New York (1993); Posttranslational Covalent Modification of Proteins, B. C. Johnson, Ed., Academic Press, New York, pp. 1-12 (1983).

Solid-phase chemical peptide synthesis methods can also be used to synthesize the polypeptide or fragments for making and/or using the chimeric monomers and polymers of the invention. Such method are known in the art, see, e.g., Merrifield (1963) J. Am. Chem. Soc. 85:2149-2154; Stewart, J. M. and Young, J. D., Solid Phase Peptide Synthesis, 2nd Ed., Pierce Chemical Co., Rockford, Ill., pp. 11-12; and have been employed in commercially available laboratory peptide design and synthesis kits (Cambridge Research Biochemicals). Such commercially available laboratory kits have generally utilized the teachings of H. M. Geysen et al, Proc. Natl. Acad. Sci., USA, 81:3998 (1984) and provide for synthesizing peptides upon the tips of a multitude of “rods” or “pins” all of which are connected to a single plate. When such a system is utilized, a plate of rods or pins is inverted and inserted into a second plate of corresponding wells or reservoirs, which contain solutions for attaching or anchoring an appropriate amino acid to the pin's or rod's tips. By repeating such a process step, i.e., inverting and inserting the rod's and pin's tips into appropriate solutions, amino acids are built into desired peptides. In addition, a number of available FMOC peptide synthesis systems are available.

Cell Matrix Binding Material

In one aspect, the chimeric polypeptides of the invention comprise a cannulae protein and a cell matrix binding motif, e.g., as a peptide or protein, e.g., an RGD (Arginine-Glycine-Aspartate) motif. The chimeric polypeptides of the invention can be designed as a cell attachment matrix material. In one aspect, monomers are constructed with two or more functionalities. In one aspect, one functionality promotes cell adhesion, for example, the inclusion of the RGD motif as a surface-exposed domain on the monomer or polymer of the invention, e.g., a nanotubule. In one aspect, other attachment domains can comprise other receptor ligands or other extracellular matrix component protein domains. In one aspect, other functionalities promote adherence of the polymer to the substrate surface, for example, promoting adherence to tissue culture dish, or cells or tissue in vitro, ex vivo or in vivo, including adhering to prostheses, bone, cartilage, teeth, metals, plastics, ceramic, etc. These functionalities can be substrate specific or non-specific; for example, poly-L-lysine domains displayed on the monomer/polymer surface. The fusion monomers possessing these individual functionalities can be blended in different ratios prior to the assembly of heteropolymeric nanotubules with the desired binding characteristics.

Chimeric polypeptides of the invention comprising a cannulae protein and a cell matrix binding motif are used in any pharmaceutical, medical device, surgical device, dental device, artificial organ, prosthesis, implant, and the like, for example, as structural elements, coating, delivery vehicle (e.g., for drugs, small molecules, antibiotics, toleragens, immunogens, antigens). Medical devices comprising a cell matrix binding motif-comprising polypeptide of the invention include dental and orthopedic pins, screws, fixtures and the like, plates, stents, stent sheaths, catheters, cannulae, tissue scaffolds, wound care devices, dressings or implants, dental devices or implants, orthopedic or dental prostheses, etc.

### Generating and Manipulating Nucleic Acids

The invention provides nucleic acids, including expression cassettes such as expression vectors, encoding the chimeric polypeptides of the invention. The invention also includes methods for modifying nucleic acids encoding the chimeric polypeptides of the invention by, e.g., synthetic ligation reassembly, optimized directed evolution system and/or saturation mutagenesis.

The nucleic acids of the invention can be made, isolated and/or manipulated by, e.g., cloning and expression of cDNA libraries, amplification of message or genomic DNA by PCR, and the like. The invention can be practiced in conjunction with any method or protocol or device known in the art, which are well described in the scientific and patent literature.

General Techniques

The nucleic acids used to practice this invention, whether RNA, iRNA, antisense nucleic acid, cDNA, genomic DNA, vectors, viruses or hybrids thereof, may be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N. Y. (1993).

Another useful means of obtaining and manipulating nucleic acids used to practice the invention is to clone from genomic samples, and, if desired, screen and re-clone inserts isolated or amplified from, e.g., genomic clones or cDNA clones. Sources of nucleic acid used in practicing the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

In one aspect, a nucleic acid encoding a polypeptide of the invention is assembled in appropriate phase with a leader sequence capable of directing secretion of the translated polypeptide or fragment thereof.

The invention provides fusion proteins and nucleic acids encoding them. In addition to chiral selection motifs, enzymes, receptors, ligands, antibodies, antigens, epitopes, cell matrix binding sites, carbohydrate binding domains, and the like, polypeptide of the invention can be fused to a heterologous peptide or polypeptide such as N-terminal identification peptide, which imparts desired characteristics such as increased stability or simplified purification. Peptides and polypeptides of the invention also can be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams (1995) Biochemistry 34:1787-1797; Dobeli (1998) Protein Expr. Purif. 12:404-414). The histidine residues facilitate detection and purification while the enterokinase cleavage site provides a means for purifying the epitope from the remainder of the fusion protein.

Transcriptional and Translational Control Sequences

The invention provides nucleic acid (e.g., DNA) sequences of the invention operatively linked to expression (e.g., transcriptional or translational) control sequence(s), e.g., promoters or enhancers, to direct or modulate RNA synthesis/expression. The expression control sequence can be in an expression vector. Exemplary bacterial promoters include lacI, lacZ, T3, T7, gpt, lambda PR, PL and trp. Exemplary eukaryotic promoters include CMV immediate early, HSV thymidine kinase, early and late SV40, LTRs from retrovirus, and mouse metallothionein I.

Promoters suitable for expressing a polypeptide in bacteria include the E. coli lac or trp promoters, the lacI promoter, the lacZ promoter, the T3 promoter, the T7 promoter, the gpt promoter, the lambda PR promoter, the lambda PL promoter, promoters from operons encoding glycolytic enzymes such as 3-phosphoglycerate kinase (PGK), and the acid phosphatase promoter. Eukaryotic promoters include the CMV immediate early promoter, the HSV thymidine kinase promoter, heat shock promoters, the early and late SV40 promoter, LTRs from retroviruses, and the mouse metallothionein-I promoter. Other promoters known to control expression of genes in prokaryotic or eukaryotic cells or their viruses may also be used.

The invention provides expression cassettes that can be expressed in a tissue-specific manner, e.g., that can express a chimeric polypeptide of the invention in a tissue-specific manner. The invention provides plants or seeds that express a chimeric polypeptide of the invention in a tissue-specific manner. The tissue-specificity can be seed specific, stem specific, leaf specific, root specific, fruit specific and the like. The nucleic acids of the invention can also be operably linked to plant promoters which are inducible upon exposure to chemicals reagents.

Expression Vectors and Cloning Vehicles

The invention provides expression vectors and cloning vehicles comprising nucleic acids of the invention, e.g., sequences encoding the chimeric polypeptides of the invention. Expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as Bacillus, Aspergillus and yeast). Vectors of the invention can include chromosomal, non-chromosomal and synthetic DNA sequences. Large numbers of suitable vectors are known to those of skill in the art, and are commercially available. Exemplary vectors are include: bacterial: pQE vectors (Qiagen), pBluescript plasmids, pNH vectors, (lambda-ZAP vectors (Stratagene); ptrc99a, pKK223-3, pDR540, pRIT2T (Pharmacia); Eukaryotic: pXT1, pSG5 (Stratagene), pSVK3, pBPV, pMSG, pSVLSV40 (Pharmacia). However, any other plasmid or other vector may be used so long as they are replicable and viable in the host. Low copy number or high copy number vectors may be employed with the present invention.

The expression vector can comprise a promoter, a ribosome binding site for translation initiation and a transcription terminator. The vector may also include appropriate sequences for amplifying expression. Mammalian expression vectors can comprise an origin of replication, any necessary ribosome binding sites, a polyadenylation site, splice donor and acceptor sites, transcriptional termination sequences, and 5′ flanking non-transcribed sequences. In some aspects, DNA sequences derived from the SV40 splice and polyadenylation sites may be used to provide the required non-transcribed genetic elements.

In one aspect, the expression vectors contain one or more selectable marker genes to permit selection of host cells containing the vector. Such selectable markers include genes encoding dihydrofolate reductase or genes conferring neomycin resistance for eukaryotic cell culture, genes conferring tetracycline or ampicillin resistance in E. coli, and the S. cerevisiae TRP1 gene. Promoter regions can be selected from any desired gene using chloramphenicol transferase (CAT) vectors or other vectors with selectable markers.

Vectors for expressing the polypeptide or fragment thereof in eukaryotic cells can also contain enhancers to increase expression levels. Examples include the SV40 enhancer on the late side of the replication origin bp 100 to 270, the cytomegalovirus early promoter enhancer, the polyoma enhancer on the late side of the replication origin, and the adenovirus enhancers.

A nucleic acid sequence can be inserted into a vector by a variety of procedures. In general, the sequence is ligated to the desired position in the vector following digestion of the insert and the vector with appropriate restriction endonucleases. Alternatively, blunt ends in both the insert and the vector may be ligated. A variety of cloning techniques are known in the art, e.g., as described in Ausubel and Sambrook. Such procedures and others are deemed to be within the scope of those skilled in the art.

The vector can be in the form of a plasmid, a viral particle, or a phage. Other vectors include chromosomal, non-chromosomal and synthetic DNA sequences, derivatives of SV40; bacterial plasmids, phage DNA, baculovirus, yeast plasmids, vectors derived from combinations of plasmids and phage DNA, viral DNA such as vaccinia, adenovirus, fowl pox virus, and pseudorabies. A variety of cloning and expression vectors for use with prokaryotic and eukaryotic hosts are described by, e.g., Sambrook. Any vector may be used as long as it is replicable and viable in the host cell.

Particular bacterial vectors which can be used include the commercially available plasmids comprising genetic elements of the well known cloning vector pBR322 (ATCC 37017), pKK223-3 (Pharmacia Fine Chemicals, Uppsala, Sweden), GEM1 (Promega Biotec, Madison, Wis., USA) pQE70, pQE60, pQE-9 (Qiagen), pD10, psiX174 pBluescript II KS, pNH8A, pNH16a, pNH18A, pNH46A (Stratagene), ptrc99a, pKK223-3, pKK233-3, DR540, pRIT5 (Pharmacia), pKK232-8 and pCM7. Particular eukaryotic vectors include pSV2CAT, pOG44, pXT1, pSG (Stratagene) pSVK3, pBPV, pMSG, and pSVL (Pharmacia). However, any other vector may be used as long as it is replicable and viable in the host cell.

The nucleic acids of the invention can be expressed in expression cassettes, vectors or viruses and transiently or stably expressed in plant cells and seeds. One exemplary transient expression system uses episomal expression systems, e.g., cauliflower mosaic virus (CaMV) viral RNA generated in the nucleus by transcription of an episomal mini-chromosome containing supercoiled DNA, see, e.g., Covey (1990) Proc. Natl. Acad. Sci. USA 87:1633-1637. Alternatively, coding sequences, i.e., all or sub-fragments of sequences of the invention can be inserted into a plant host cell genome becoming an integral part of the host chromosomal DNA. Sense or antisense transcripts can be expressed in this manner. A vector comprising the sequences (e.g., promoters or coding regions) from nucleic acids of the invention can comprise a marker gene that confers a selectable phenotype on a plant cell or a seed. For example, the marker may encode biocide resistance, particularly antibiotic resistance, such as resistance to kanamycin, G418, bleomycin, hygromycin, or herbicide resistance, such as resistance to chlorosulfuron or Basta.

Expression vectors capable of expressing nucleic acids and proteins in plants are well known in the art, and can include, e.g., vectors from Agrobacterium spp., potato virus X (see, e.g., Angell (1997) EMBO J. 16:3675-3684), tobacco mosaic virus (see, e.g., Casper (1996) Gene 173:69-73), tomato bushy stunt virus (see, e.g., Hillman (1989) Virology 169:42-50), tobacco etch virus (see, e.g., Dolja (1997) Virology 234:243-252), bean golden mosaic virus (see, e.g., Morinaga (1993) Microbiol Immunol. 37:471-476), cauliflower mosaic virus (see, e.g., Cecchini (1997) Mol. Plant. Microbe Interact. 10:1094-1101), maize Ac/Ds transposable element (see, e.g., Rubin (1997) Mol. Cell. Biol. 17:6294-6302; Kunze (1996) Curr. Top. Microbiol. Immunol. 204:161-194), and the maize suppressor-mutator (Spm) transposable element (see, e.g., Schlappi (1996) Plant Mol. Biol. 32:717-725); and derivatives thereof.

In one aspect, the expression vector can have two replication systems to allow it to be maintained in two organisms, for example in mammalian or insect cells for expression and in a prokaryotic host for cloning and amplification. Furthermore, for integrating expression vectors, the expression vector can contain at least one sequence homologous to the host cell genome. It can contain two homologous sequences which flank the expression construct. The integrating vector can be directed to a specific locus in the host cell by selecting the appropriate homologous sequence for inclusion in the vector. Constructs for integrating vectors are well known in the art.

Expression vectors of the invention may also include a selectable marker gene to allow for the selection of bacterial strains that have been transformed, e.g., genes which render the bacteria resistant to drugs such as ampicillin, chloramphenicol, erythromycin, kanamycin, neomycin and tetracycline. Selectable markers can also include biosynthetic genes, such as those in the histidine, tryptophan and leucine biosynthetic pathways.

The terms “vector” and “expression cassette” as used herein can be used interchangeably and refer to a nucleotide sequence which is capable of affecting expression of a nucleic acid, e.g., a mutated nucleic acid of the invention. Expression cassettes can include at least a promoter operably linked with the polypeptide coding sequence; and, optionally, with other sequences, e.g., transcription termination signals. Additional factors necessary or helpful in effecting expression may also be used, e.g., enhancers. “Operably linked” as used herein refers to linkage of a promoter upstream from a DNA sequence such that the promoter mediates transcription of the DNA sequence. Thus, expression cassettes also include plasmids, expression vectors, recombinant viruses, any form of recombinant “naked DNA” vector, and the like. A “vector” comprises a nucleic acid which can infect, transfect, transiently or permanently transduce a cell. It will be recognized that a vector call be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and includes both the expression and non-expression plasmids.

The appropriate DNA sequence may be inserted into the vector by a variety of procedures. In general, the DNA sequence is ligated to the desired position in the vector following digestion of the insert and the vector with appropriate restriction endonucleases. Alternatively, blunt ends in both the insert and the vector may be ligated. A variety of cloning techniques are disclosed in Ausubel et al. Current Protocols in Molecular Biology, John Wiley 503 Sons, Inc. 1997 and Sambrook et al., Molecular Cloning: A Laboratory Manual 2nd Ed., Cold Spring Harbor Laboratory Press (1989. Such procedures and others are deemed to be within the scope of those skilled in the art.

The vector may be, for example, in the form of a plasmid, a viral particle, or a phage. Other vectors include chromosomal, nonchromosomal and synthetic DNA sequences, derivatives of SV40; bacterial plasmids, phage DNA, baculovirus, yeast plasmids, vectors derived from combinations of plasmids and phage DNA, viral DNA such as vaccinia, adenovirus, fowl pox virus and pseudorabies. A variety of cloning and expression vectors for use with prokaryotic and eukaryotic hosts are described by Sambrook, et al., Molecular Cloning: A Laboratory Manual, 2nd Ed., Cold Spring Harbor, N.Y., (1989).

Host Cells and Transformed Cells

The invention also provides a transformed cell comprising a nucleic acid sequence of the invention, e.g., a sequence encoding chimeric polypeptides of the invention, or an expression cassette, e.g., a vector, of the invention. The host cell may be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Lactococcus lactis, Streptomyces, Bacillus subtilis, Bacillus cereus, Salmonella typhimurium or any species within the genera Bacillus, Streptomyces and Staphylococcus. Exemplary insect cells include Drosophila S2 and Spodoptera Sf9. Exemplary yeast cells include Pichia pastoris, Saccharomyces cerevisiae or Schizosaccharomyces pombe. Exemplary animal cells include CHO, COS or Bowes melanoma or any mouse or human cell line. The selection of an appropriate host is within the abilities of those skilled in the art. Techniques for transforming a wide variety of higher plant species are well known and described in the technical and scientific literature. See, e.g., Weising (1988) Ann. Rev. Genet. 22:421-477, U.S. Pat. No. 5,750,870.

The vector can be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation (Davis, L., Dibner, M., Battey, I., Basic Methods in Molecular Biology, (1986)).

In one aspect, the nucleic acids or vectors of the invention are introduced into the cells for screening, thus, the nucleic acids enter the cells in a manner suitable for subsequent expression of the nucleic acid. The method of introduction is largely dictated by the targeted cell type. Exemplary methods include CaPO₄ precipitation, liposome fusion, lipofection (e.g., LIPOFECTIN™), electroporation, viral infection, etc. The candidate nucleic acids may stably integrate into the genome of the host cell (for example, with retroviral introduction) or may exist either transiently or stably in the cytoplasm (i.e. through the use of traditional plasmids, utilizing standard regulatory sequences, selection markers, etc.). As many pharmaceutically important screens require human or model mammalian cell targets, retroviral vectors capable of transfecting such targets are preferred.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment thereof can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

Host cells containing the polynucleotides of interest, e.g., nucleic acids of the invention, can be cultured in conventional nutrient media modified as appropriate for activating promoters, selecting transformants or amplifying genes. The culture conditions, such as temperature, pH and the like, are those previously used with the host cell selected for expression and will be apparent to the ordinarily skilled artisan. The clones which are identified as having the specified enzyme activity may then be sequenced to identify the polynucleotide sequence encoding an enzyme having the enhanced activity.

The invention provides a method for overexpressing a recombinant glucanase in a cell comprising expressing a vector comprising a nucleic acid of the invention, e.g., a nucleic acid comprising a nucleic acid sequence with at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to an exemplary sequence of the invention over a region of at least about 100 residues, wherein the sequence identities are determined by analysis with a sequence comparison algorithm or by visual inspection, or, a nucleic acid that hybridizes under stringent conditions to a nucleic acid sequence of the invention. The overexpression can be effected by any means, e.g., use of a high activity promoter, a dicistronic vector or by gene amplification of the vector.

The nucleic acids of the invention can be expressed, or overexpressed, in any in vitro or in vivo expression system. Any cell culture systems can be employed to express, or over-express, recombinant protein, including bacterial, insect, yeast, fungal or mammalian cultures. Over-expression can be effected by appropriate choice of promoters, enhancers, vectors (e.g., use of replicon vectors, dicistronic vectors (see, e.g., Gurtu (1996) Biochem. Biophys. Res. Commun. 229:295-8), media, culture systems and the like. In one aspect, gene amplification using selection markers, e.g., glutamine synthetase (see, e.g., Sanders (1987) Dev. Biol. Stand. 66:55-63), in cell systems are used to overexpress the polypeptides of the invention.

Additional details regarding this approach are in the public literature and/or are known to the skilled artisan. In a particular non-limiting exemplification, such publicly available literature includes EP 0659215 (WO 9403612 A1) (Nevalainen et al.); Lapidot, A., Mechaly, A., Shoham, Y., “Overexpression and single-step purification of a thermostable glucanase from Bacillus stearothermophilus T-6,” J. Biotechnol. November 51:259-64 (1996); Lüthi, E., Jasmat, N. B., Bergquist, P. L., “Endoglucanase from the extremely thermophilic bacterium Caldocellum saccharolyticum: overexpression of the gene in Escherichia coli and characterization of the gene product,” Appl. Environ. Microbiol. Sep 56:2677-83 (1990); and Sung, W. L., Luk, C. K., Zahab, D. M., Wakarchuk, W., “Overexpression of the Bacillus subtilis and circulans endoglucanases in Escherichia coli,” Protein Expr. Purif. June 4:200-6 (1993), although these references do not teach the inventive enzymes of the instant application.

The host cell may be any of the host cells familiar to those skilled in the art, including prokaryotic cells, eukaryotic cells, mammalian cells, insect cells, or plant cells. As representative examples of appropriate hosts, there may be mentioned: bacterial cells, such as E. coli, Streptomyces, Bacillus (e.g., Bacillus subtilis, Bacillus ceres), Salmonella typhimurium and various species within the genera Streptomyces and Staphylococcus, fungal cells, such as yeast, insect cells such as Drosophila S2 and Spodoptera Sf9, animal cells such as CHO, COS or Bowes melanoma and adenoviruses. The selection of an appropriate host is within the abilities of those skilled in the art.

Amplification of Nucleic Acids

In practicing the invention, nucleic acids of the invention and nucleic acids encoding the chimeric polypeptides of the invention, or modified nucleic acids of the invention, can be reproduced by amplification. Amplification can also be used to clone or modify the nucleic acids of the invention. Thus, the invention provides amplification primer sequence pairs for amplifying nucleic acids of the invention.

Amplification reactions can also be used to quantify the amount of nucleic acid in a sample (such as the amount of message in a cell sample), label the nucleic acid (e.g., to apply it to an array or a blot), detect the nucleic acid, or quantify the amount of a specific nucleic acid in a sample. In one aspect of the invention, message isolated from a cell or a cDNA library are amplified.

The skilled artisan can select and design suitable oligonucleotide amplification primers. Amplification methods are also well known in the art, and include, e.g., polymerase chain reaction, PCR (see, e.g., PCR PROTOCOLS, A GUIDE TO METHODS AND APPLICATIONS, ed. Innis, Academic Press, N.Y. (1990) and PCR STRATEGIES (1995), ed. Innis, Academic Press, Inc., N.Y., ligase chain reaction (LCR) (see, e.g., Wu (1989) Genomics 4:560; Landegren (1988) Science 241:1077; Barringer (1990) Gene 89:117); transcription amplification (see, e.g., Kwoh (1989) Proc. Natl. Acad. Sci. USA 86:1173); and, self-sustained sequence replication (see, e.g., Guatelli (1990) Proc. Natl. Acad. Sci. USA 87:1874); Q Beta replicase amplification (see, e.g., Smith (1997) J. Clin. Microbiol. 35:1477-1491), automated Q-beta replicase amplification assay (see, e.g., Burg (1996) Mol. Cell. Probes 10:257-271) and other RNA polymerase mediated techniques (e.g., NASBA, Cangene, Mississauga, Ontario); see also Berger (1987) Methods Enzymol. 152:307-316; Sambrook; Ausubel; U.S. Pat. Nos. 4,683,195 and 4,683,202; Sooknanan (1995) Biotechnology 13:563-564.

### Determining the Degree of Sequence Identity

The cannulae polypeptide can comprise a protein having at least about 55%, 60%, 65%, 70%, 75%, 80%, 85%, 90%, 95%, 98%, 99%, or more, sequence identity to SEQ ID NO:2, SEQ ID NO:4, SEQ ID NO:6, SEQ ID NO:8, SEQ ID NO:10 or SEQ ID NO:12 and is capable of assembling into a polymer, e.g., a nanotubule, or, is capable of acting as a chiral selector. The chimeric cannulae proteins can assemble into nanotubular polymers to act as a chiral selectors, biosynthetic pathways, selection scaffoldings and the like. The extent of sequence identity (homology) may be determined using any computer program and associated parameters, including those described herein, such as BLAST 2.2.2. or FASTA version 3.0t78, with the default parameters.

Various sequence comparison programs identified herein are used in this aspect of the invention. Protein and/or nucleic acid sequence identities (homologies) may be evaluated using any of the variety of sequence comparison algorithms and programs known in the art. Such algorithms and programs include, but are not limited to, TBLASTN, BLASTP, FASTA, TFASTA, and CLUSTALW (Pearson and Lipman, Proc. Natl. Acad. Sci. USA 85(8):2444-2448,1988; Altschul et al., J. Mol. Biol. 215(3):403-410, 1990; Thompson et al., Nucleic Acids Res. 22(2):4673-4680, 1994; Higgins et al., Methods Enzymol. 266:383-402, 1996; Altschul et al., J. Mol. Biol. 215(3):403-410, 1990; Altschul et al., Nature Genetics 3:266-272, 1993).

Homology or identity can be measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of homology to various deletions, substitutions and other modifications. The terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. For sequence comparison, one sequence can act as a reference sequence (a sequence of the invention to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the numbers of contiguous residues. For example, in alternative aspects of the invention, contiguous residues ranging anywhere from 20 to the full length of an exemplary polypeptide or nucleic acid sequence of the invention are compared to a reference sequence of the same number of contiguous positions after the two sequences are optimally aligned. If the reference sequence has the requisite sequence identity to an exemplary polypeptide or nucleic acid sequence of the invention, e.g., 50%, 55%, 60%, 65%, 70%, 75%, 80%, 90% or 95%, 98%, 99% or more sequence identity to a cannulae polypeptide, that sequence may be within the scope of the invention. In alternative embodiments, subsequences ranging from about 20 to 600, about 50 to 200, and about 100 to 150 are compared to a reference sequence of the same number of contiguous positions after the two sequences are optimally aligned. Methods of alignment of sequence for comparison are well known in the art. Optimal alignment of sequences for comparison can be conducted, e.g., by the local homology algorithm of Smith & Waterman, Adv. Appl. Math. 2:482, 1981, by the homology alignment algorithm of Needleman & Wunsch, J. Mol. Biol. 48:443, 1970, by the search for similarity method of person & Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection. Other algorithms for determining homology or identity include, for example, in addition to a BLAST program (Basic Local Alignment Search Tool at the National Center for Biological Information), ALIGN, AMAS (Analysis of Multiply Aligned Sequences), AMPS (Protein Multiple Sequence Alignment), ASSET (Aligned Segment Statistical Evaluation Tool), BANDS, BESTSCOR, BIOSCAN (Biological Sequence Comparative Analysis Node), BLIMPS (BLocks IMProved Searcher), FASTA, Intervals & Points, BMB, CLUSTAL V, CLUSTAL W, CONSENSUS, LCONSENSUS, WCONSENSUS, Smith-Waterman algorithm, DARWIN, Las Vegas algorithm, FNAT (Forced Nucleotide Alignment Tool), Framealign, Framesearch, DYNAMIC, FILTER, FSAP (Fristensky Sequence Analysis Package), GAP (Global Alignment Program), GENAL, GIBBS, GenQuest, ISSC (Sensitive Sequence Comparison), LALIGN (Local Sequence Alignment), LCP (Local Content Program), MACAW (Multiple Alignment Construction & Analysis Workbench), MAP (Multiple Alignment Program), MBLKP, MBLKN, PIMA (Pattern-Induced Multi-sequence Alignment), SAGA (Sequence Alignment by Genetic Algorithm) and WHAT-IF. Such alignment programs can also be used to screen genome databases to identify polynucleotide sequences having substantially identical sequences. A number of genome databases are available, for example, a substantial portion of the human genome is available as part of the Human Genome Sequencing Project (Gibbs, 1995). Databases containing genomic information annotated with some functional information are maintained by different organization, and are accessible via the internet.

BLAST, BLAST 2.0 and BLAST 2.2.2 algorithms are also used to practice the invention. They are described, e.g., in Altschul (1977) Nuc. Acids Res. 25:3389-3402; Altschul (1990) J. Mol. Biol. 215:403-410. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul (1990) supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectations (E) of 10, and the BLOSUM62 scoring matrix (see Henikoff & Henikoff (1989) Proc. Natl. Acad. Sci. USA 89:10915) alignments (B) of 50, expectation (E) of 10, M=5, N=−4, and a comparison of both strands. The BLAST algorithm also performs a statistical analysis of the similarity between two sequences (see, e.g., Karlin & Altschul (1993) Proc. Natl. Acad. Sci. USA 90:5873). One measure of similarity provided by BLAST algorithm is the smallest sum probability (P(N)), which provides an indication of the probability by which a match between two nucleotide or amino acid sequences would occur by chance. For example, a nucleic acid is considered similar to a references sequence if the smallest sum probability in a comparison of the test nucleic acid to the reference nucleic acid is less than about 0.2, more preferably less than about 0.01, and most preferably less than about 0.001. In one aspect, protein and nucleic acid sequence homologies are evaluated using the Basic Local Alignment Search Tool (“BLAST”). For example, five specific BLAST programs can be used to perform the following task: (1) BLASTP and BLAST3 compare an amino acid query sequence against a protein sequence database; (2) BLASTN compares a nucleotide query sequence against a nucleotide sequence database; (3) BLASTX compares the six-frame conceptual translation products of a query nucleotide sequence (both strands) against a protein sequence database; (4) TBLASTN compares a query protein sequence against a nucleotide sequence database translated in all six reading frames (both strands); and, (5) TBLASTX compares the six-frame translations of a nucleotide query sequence against the six-frame translations of a nucleotide sequence database. The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., Science 256:1443-1445, 1992; Henikoff and Henikoff, Proteins 17:49-61, 1993). Less preferably, the PAM or PAM250 matrices may also be used (see, e.g., Schwartz and Dayhoff, eds., 1978, Matrices for Detecting Distance Relationships: Atlas of Protein Sequence and Structure, Washington: National Biomedical Research Foundation).

In one aspect of the invention, to determine if a nucleic acid has the requisite sequence identity to be within the scope of the invention, the NCBI BLAST 2.2.2 programs is used, default options to blastp. There are about 38 setting options in the BLAST 2.2.2 program. In this exemplary aspect of the invention, all default values are used except for the default filtering setting (i.e., all parameters set to default except filtering which is set to OFF); in its place a “—F F” setting is used, which disables filtering. Use of default filtering often results in Karlin-Altschul violations due to short length of sequence.

The default values used in this exemplary aspect of the invention include:

“Filter for low complexity: ON

Word Size: 3

Matrix: Blosum62

Gap Costs Existence: 11

Extension: 1”

Other default settings can be: filter for low complexity OFF, word size of 3 for protein, BLOSUM62 matrix, gap existence penalty of −11 and a gap extension penalty of −1. An exemplary NCBI BLAST 2.2.2 program setting has the “—W” option default to 0. This means that, if not set, the word size defaults to 3 for proteins and 11 for nucleotides.

### Modification of Nucleic Acids

The invention provides methods of generating variants of the nucleic acids encoding the chimeric polypeptides of the invention. These methods can be repeated or used in various combinations to generate chimeric polypeptides having an altered or different activity or an altered or different stability from that of a chimeric polypeptide encoded by the template nucleic acid. These methods also can be repeated or used in various combinations, e.g., to generate variations in gene/message expression, message translation or message stability. In another aspect, the genetic composition of a cell is altered by, e.g., modification of a homologous gene ex vivo, followed by its reinsertion into the cell.

The invention provides methods for evolving enzymes in vitro or in vivo to produce variants with characteristics tailored for specific applications. For example, using the evolution strategies of the invention, enzyme active sites can be modified to produce proteins that retain stereospecific substrate recognition but lack catalytic activity. In one aspect, the chimeric monomers and polymers of the invention are evolved for applications in chiral selection using targeted mutagenesis and in vitro evolution strategies, e.g., as described herein, such as Gene Site Saturation Mutagenesis (GSSM™) and GeneReassembly™ (see, e.g., U.S. Pat. Nos. 6,171,820, and 5,965,408 respectively). These technologies are used to create large libraries of mutagenized sequence variants that are screened in a high throughput (HT) assay that selects mutants with a desired phenotype.

With GSSM™, the effects of all 64 codons (even nonsense codons) can be tested at each triplet position along the entire length of the open reading frame of the gene being analyzed. For example, in the case of a 200 amino acid protein, the gene can be simultaneously assembled in 200 different reaction tubes where all 64 codons are present during the synthesis of each amino acid. The result is a library of single point mutants with all possible codons represented at each position of the open reading frame. The library of GSSM™ variants then can be screened using a HT assay to identify variants that have evolved the target phenotype. Individual GSSM™ variants that exhibit the desired property then can be further evolved using GeneReassembly™.

In GeneReassembly™, a new library of mutants can be constructed by recombining DNA fragments taken from the single point mutant sequences identified in the GSSM™ screen. Therefore, the reassembly library can contain open reading frames that contain multiple point mutations that have accumulated as a result of the recombination process. The reassembled variants can be screened to identify mutant combinations with further improvements in the target activity. If necessary, GeneReassembly™ can be repeated until an evolved protein with the desired target properties is identified. These protein evolution strategies do not require prior knowledge of protein structure and therefore produce unbiased pools of protein variants for screening.

In one aspect, the invention provides combinatorial approaches to chiral selector methods. For example, high throughput screening methods of the invention can be used to screen libraries of peptides to identify those sequences with unique enantio-recognition properties; see, e.g., Chankvetadze (2001) supra. Thus, the invention provides chimeric monomers and polymers, including nanotubules, comprising libraries of peptides. In one aspect, these peptide sequences are inserted into the sequence of a chimeric monomer and uniformly displayed on the nanotubule surface.

In one aspect, to apply evolution technologies to the development of chiral selectors, the invention provides a high throughput screen suitable for the identification of protein variants that possess increased enantioselectivity. For example, Henriksson (1996) supra, have reported that the activity of cellobiohydrolase (CBH) from Trichoderma reesei is differentially inhibited by the (R)- and (S)-enantiomers of the beta-blockers propanolol and alprenolol. The T. reesei CBH has been demonstrated to be an effective chiral selector for beta-blockers and the chiral selectivity is consistent with the inhibition data. Based on these results, the methods of the invention evolve the enantioselectivity of CBH using evolution strategies. In one aspect, a high throughput screen is used that measures enantiospecific inhibition of CBH activity.

In practicing the invention, a nucleic acid (e.g., a nucleic acid encoding a chimeric polypeptide of the invention) can be altered by any means. For example, random or stochastic methods, or, non-stochastic, or “directed evolution,” methods, see, e.g., U.S. Pat. No. 6,361,974. Methods for random mutation of genes are well known in the art, see, e.g., U.S. Pat. No. 5,830,696. For example, mutagens can be used to randomly mutate a gene. Mutagens include, e.g., ultraviolet light or gamma irradiation, or a chemical mutagen, e.g., mitomycin, nitrous acid, photoactivated psoralens, alone or in combination, to induce DNA breaks amenable to repair by recombination. Other chemical mutagens include, for example, sodium bisulfite, nitrous acid, hydroxylamine, hydrazine or formic acid. Other mutagens are analogues of nucleotide precursors, e.g., nitrosoguanidine, 5-bromouracil, 2-aminopurine, or acridine. These agents can be added to a PCR reaction in place of the nucleotide precursor thereby mutating the sequence. Intercalating agents such as proflavine, acriflavine, quinacrine and the like can also be used.

Any technique in molecular biology can be used, e.g., random PCR mutagenesis, see, e.g., Rice (1992) Proc. Natl. Acad. Sci. USA 89:5467-5471; or, combinatorial multiple cassette mutagenesis, see, e.g., Crameri (1995) Biotechniques 18:194-196. Alternatively, nucleic acids, e.g., genes, can be reassembled after random, or “stochastic,” fragmentation, see, e.g., U.S. Pat. Nos. 6,291,242; 6,287,862; 6,287,861; 5,955,358; 5,830,721; 5,824,514; 5,811,238; 5,605,793. In alternative aspects, modifications, additions or deletions are introduced by error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, gene site saturated mutagenesis (GSSM™, synthetic ligation reassembly (SLR), recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, and/or a combination of these and other methods.

The following publications describe a variety of recursive recombination procedures and/or methods which can be incorporated into the methods of the invention: Stemmer (1999) “Molecular breeding of viruses for targeting and other clinical properties” Tumor Targeting 4:1-4; Ness (1999) Nature Biotechnology 17:893-896; Chang (1999) “Evolution of a cytokine using DNA family shuffling” Nature Biotechnology 17:793-797; Minshull (1999) “Protein evolution by molecular breeding” Current Opinion in Chemical Biology 3:284-290; Christians (1999) “Directed evolution of thymidine kinase for AZT phosphorylation using DNA family shuffling” Nature Biotechnology 17:259-264; Crameri (1998) “DNA shuffling of a family of genes from diverse species accelerates directed evolution” Nature 391:288-291; Crameri (1997) “Molecular evolution of an arsenate detoxification pathway by DNA shuffling,” Nature Biotechnology 15:436-438; Zhang (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp. 447-457; Crameri and Stemmer (1995) “Combinatorial multiple cassette mutagenesis creates all the permutations of mutant and wildtype cassettes” BioTechniques 18:194-195; Stemmer et al. (1995) “Single-step assembly of a gene and entire plasmid form large numbers of oligodeoxyribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Mutational methods of generating variant sequences in practicing the methods of the invention include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional protocols for generating variant sequences in practicing the methods of the invention include point mismatch repair (Kramer (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh (1986) “Use of oligonucleotides to generate large deletions” Nucl. Acids Res. 14: 5115), restriction-selection and restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and Grundstrom et al. (1985) “Oligonucleotide-directed mutagenesis by microscale ‘shot-gun’ gene synthesis” Nucl. Acids Res. 13: 3305-3316), double-strand break repair (Mandecki (1986); Arnold (1993) “Protein engineering for unusual environments” Current Opinion in Biotechnology 4:450-455. “Oligonucleotide-directed double-strand break repair in plasmids of Escherichia coli: a method for site-specific mutagenesis” Proc. Natl. Acad. Sci. USA, 83:7177-7181). Additional details on many of the above methods can be found in Methods in Enzymology Volume 154, which also describes useful controls for trouble-shooting problems with various mutagenesis methods.

Additional protocols for generating variant sequences in practicing the methods of the invention include those discussed in U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;”” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination; “WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination.”

Additional protocols for generating variant sequences in practicing the methods of the invention are described in U.S. patent application Ser. No. 09/407,800, “SHUFFLING OF CODON ALTERED GENES” by Patten et al. filed Sep. 28, 1999; “EVOLUTION OF WHOLE CELLS AND ORGANISMS BY RECURSIVE SEQUENCE RECOMBINATION” by del Cardayre et al., U.S. Pat. No. 6,379,964; “OLIGONUCLEOTIDE MEDIATED NUCLEIC ACID RECOMBINATION” by Crameri et al., U.S. Pat. Nos. 6,319,714; 6,368,861; 6,376,246; 6,423,542; 6,426,224 and PCT/US00/01203; “USE OF CODON-VARIED OLIGONUCLEOTIDE SYNTHESIS FOR SYNTHETIC SHUFFLING” by Welch et al., U.S. Pat. No. 6,436,675; “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202) and, e.g. “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579); “METHODS OF POPULATING DATA STRUCTURES FOR USE IN EVOLUTIONARY SIMULATIONS” by Selifonov and Stemmer, filed Jan. 18, 2000 (PCT/US00/01138); and “SINGLE-STRANDED NUCLEIC ACID TEMPLATE-MEDIATED RECOMBINATION AND NUCLEIC ACID FRAGMENT ISOLATION” by Affholter, filed Sep. 6, 2000 (U.S. Ser. No. 09/656,549); and U.S. Pat. Nos. 6,177,263; 6,153,410.

### Non-Stochastic, or “Directed Evolution,” Methods

Exemplary protocols for generating variant sequences (e.g., modified sequences encoding chimeric polypeptides of the invention) in practicing the methods of the invention include non-stochastic, or “directed evolution,” methods, such as, e.g., saturation mutagenesis (GSSM™, synthetic ligation reassembly (SLR), or a combination thereof. These methods can be used to modify the nucleic acids to generate chimeric polypeptides with new or altered properties (e.g., chiral selection activity under high or low acidic or alkaline conditions, high or low temperatures, high or low salt conditions and the like; different substrate affinity; enantioselective activity; modified antibody binding activity, etc.). Polypeptides encoded by the modified nucleic acids can be screened for an activity before testing for proteolytic or other activity. Any testing modality or protocol can be used, e.g., using a capillary array platform. See, e.g., U.S. Pat. Nos. 6,361,974; 6,280,926; 5,939,250.

Saturation Mutagenesis, or, GSSM™

In one aspect of the invention, non-stochastic gene modification, a “directed evolution process,” is used to generate modified sequences encoding chimeric polypeptides of the invention with new or altered properties. Variations of this method have been termed “Gene Site-Saturation Mutagenesis™ (GSSM™, “site-saturation mutagenesis,” “saturation mutagenesis” or simply “GSSM™” It can be used in combination with other mutagenization processes. See, e.g., U.S. Pat. Nos. 6,171,820; 6,238,884. In one aspect, GSSM™ comprises providing a template polynucleotide and a plurality of oligonucleotides, wherein each oligonucleotide comprises a sequence homologous to the template polynucleotide, thereby targeting a specific sequence of the template polynucleotide, and a sequence that is a variant of the homologous gene; generating progeny polynucleotides comprising non-stochastic sequence variations by replicating the template polynucleotide with the oligonucleotides, thereby generating polynucleotides comprising homologous gene sequence variations.

In one aspect, codon primers containing a degenerate N,N,G/T sequence are used to introduce point mutations into a polynucleotide, so as to generate a set of progeny polypeptides in which a full range of single amino acid substitutions is represented at each amino acid position, e.g., an amino acid residue in an enzyme active site or ligand binding site targeted to be modified. These oligonucleotides can comprise a contiguous first homologous sequence, a degenerate N,N,G/T sequence, and, optionally, a second homologous sequence. The downstream progeny translational products from the use of such oligonucleotides include all possible amino acid changes at each amino acid site along the polypeptide, because the degeneracy of the N,N,G/T sequence includes codons for all 20 amino acids. In one aspect, one such degenerate oligonucleotide (comprised of, e.g., one degenerate N,N,G/T cassette) is used for subjecting each original codon in a parental polynucleotide template to a full range of codon substitutions. In another aspect, at least two degenerate cassettes are used—either in the same oligonucleotide or not, for subjecting at least two original codons in a parental polynucleotide template to a full range of codon substitutions. For example, more than one N,N,G/T sequence can be contained in one oligonucleotide to introduce amino acid mutations at more than one site. This plurality of N,N,G/T sequences can be directly contiguous, or separated by one or more additional nucleotide sequence(s). In another aspect, oligonucleotides serviceable for introducing additions and deletions can be used either alone or in combination with the codons containing an N,N,G/T sequence, to introduce any combination or permutation of amino acid additions, deletions, and/or substitutions.

In one aspect, simultaneous mutagenesis of two or more contiguous amino acid positions is done using an oligonucleotide that contains contiguous N,N,G/T triplets, i.e. a degenerate (N,N,G/T)n sequence. In another aspect, degenerate cassettes having less degeneracy than the N,N,G/T sequence are used. For example, it may be desirable in some instances to use (e.g. in an oligonucleotide) a degenerate triplet sequence comprised of only one N, where said N can be in the first second or third position of the triplet. Any other bases including any combinations and permutations thereof can be used in the remaining two positions of the triplet. Alternatively, it may be desirable in some instances to use (e.g. in an oligo) a degenerate N,N,N triplet sequence.

In one aspect, use of degenerate triplets (e.g., N,N,G/T triplets) allows for systematic and easy generation of a full range of possible natural amino acids (for a total of 20 amino acids) into each and every amino acid position in a polypeptide (in alternative aspects, the methods also include generation of less than all possible substitutions per amino acid residue, or codon, position). For example, for a 100 amino acid polypeptide, 2000 distinct species (i.e. 20 possible amino acids per position X 100 amino acid positions) can be generated. Through the use of an oligonucleotide or set of oligonucleotides containing a degenerate N,N,G/T triplet, 32 individual sequences can code for all 20 possible natural amino acids. Thus, in a reaction vessel in which a parental polynucleotide sequence is subjected to saturation mutagenesis using at least one such oligonucleotide, there are generated 32 distinct progeny polynucleotides encoding 20 distinct polypeptides. In contrast, the use of a non-degenerate oligonucleotide in site-directed mutagenesis leads to only one progeny polypeptide product per reaction vessel. Nondegenerate oligonucleotides can optionally be used in combination with degenerate primers disclosed; for example, nondegenerate oligonucleotides can be used to generate specific point mutations in a working polynucleotide. This provides one means to generate specific silent point mutations, point mutations leading to corresponding amino acid changes, and point mutations that cause the generation of stop codons and the corresponding expression of polypeptide fragments.

In one aspect, each saturation mutagenesis reaction vessel contains polynucleotides encoding at least 20 progeny polypeptide molecules such that all 20 natural amino acids are represented at the one specific amino acid position corresponding to the codon position mutagenized in the parental polynucleotide (other aspects use less than all 20 natural combinations). The 32-fold degenerate progeny polypeptides generated from each saturation mutagenesis reaction vessel can be subjected to clonal amplification (e.g. cloned into a suitable host, e.g., E. coli host, using, e.g., an expression vector) and subjected to expression screening. When an individual progeny polypeptide is identified by screening to display a favorable change in property (when compared to the parental polypeptide, such as increased proteolytic activity under alkaline or acidic conditions), it can be sequenced to identify the correspondingly favorable amino acid substitution contained therein.

In one aspect, upon mutagenizing each and every amino acid position in a parental polypeptide using saturation mutagenesis as disclosed herein, favorable amino acid changes may be identified at more than one amino acid position. One or more new progeny molecules can be generated that contain a combination of all or part of these favorable amino acid substitutions. For example, if 2 specific favorable amino acid changes are identified in each of 3 amino acid positions in a polypeptide, the permutations include 3 possibilities at each position (no change from the original amino acid, and each of two favorable changes) and 3 positions. Thus, there are 3×3×3 or 27 total possibilities, including 7 that were previously examined −6 single point mutations (i.e. 2 at each of three positions) and no change at any position.

In another aspect, site-saturation mutagenesis can be used together with another stochastic or non-stochastic means to vary sequence, e.g., synthetic ligation reassembly (see below), shuffling, chimerization, recombination and other mutagenizing processes and mutagenizing agents. This invention provides for the use of any mutagenizing process(es), including saturation mutagenesis, in an iterative manner.

Synthetic Ligation Reassembly (SLR)

In practicing the methods of the invention a non-stochastic gene modification system termed “synthetic ligation reassembly,” or simply “SLR,” a “directed evolution process,” can be used to generate modified sequences encoding chimeric polypeptides of the invention with new or altered properties. SLR is a method of ligating oligonucleotide fragments together non-stochastically. This method differs from stochastic oligonucleotide shuffling in that the nucleic acid building blocks are not shuffled, concatenated or chimerized randomly, but rather are assembled non-stochastically. See, e.g., U.S. patent application Ser. No. 09/332,835 entitled “Synthetic Ligation Reassembly in Directed Evolution” and filed on Jun. 14, 1999 (“U.S. Ser. No. 09/332,835”). In one aspect, SLR comprises the following steps: (a) providing a template polynucleotide, wherein the template polynucleotide comprises sequence encoding a homologous gene; (b) providing a plurality of building block polynucleotides, wherein the building block polynucleotides are designed to cross-over reassemble with the template polynucleotide at a predetermined sequence, and a building block polynucleotide comprises a sequence that is a variant of the homologous gene and a sequence homologous to the template polynucleotide flanking the variant sequence; (c) combining a building block polynucleotide with a template polynucleotide such that the building block polynucleotide cross-over reassembles with the template polynucleotide to generate polynucleotides comprising homologous gene sequence variations.

SLR does not depend on the presence of high levels of homology between polynucleotides to be rearranged. Thus, this method can be used to non-stochastically generate libraries (or sets) of progeny molecules comprised of over 10100 different chimeras. SLR can be used to generate libraries comprised of over 101000 different progeny chimeras. Thus, aspects of the present invention include non-stochastic methods of producing a set of finalized chimeric nucleic acid molecule shaving an overall assembly order that is chosen by design. This method includes the steps of generating by design a plurality of specific nucleic acid building blocks having serviceable mutually compatible ligatable ends, and assembling these nucleic acid building blocks, such that a designed overall assembly order is achieved.

The mutually compatible ligatable ends of the nucleic acid building blocks to be assembled are considered to be “serviceable” for this type of ordered assembly if they enable the building blocks to be coupled in predetermined orders. Thus, the overall assembly order in which the nucleic acid building blocks can be coupled is specified by the design of the ligatable ends. If more than one assembly step is to be used, then the overall assembly order in which the nucleic acid building blocks can be coupled is also specified by the sequential order of the assembly step(s). In one aspect, the annealed building pieces are treated with an enzyme, such as a ligase (e.g. T4 DNA ligase), to achieve covalent bonding of the building pieces.

In one aspect, the design of the oligonucleotide building blocks is obtained by analyzing a set of progenitor nucleic acid sequence templates that serve as a basis for producing a progeny set of finalized chimeric polynucleotides. These parental oligonucleotide templates thus serve as a source of sequence information that aids in the design of the nucleic acid building blocks that are to be mutagenized, e.g., chimerized or shuffled. In one aspect of this method, the sequences of a plurality of parental nucleic acid templates are aligned in order to select one or more demarcation points. The demarcation points can be located at an area of homology, and are comprised of one or more nucleotides. These demarcation points are preferably shared by at least two of the progenitor templates. The demarcation points can thereby be used to delineate the boundaries of oligonucleotide building blocks to be generated in order to rearrange the parental polynucleotides. The demarcation points identified and selected in the progenitor molecules serve as potential chimerization points in the assembly of the final chimeric progeny molecules. A demarcation point can be an area of homology (comprised of at least one homologous nucleotide base) shared by at least two parental polynucleotide sequences. Alternatively, a demarcation point can be an area of homology that is shared by at least half of the parental polynucleotide sequences, or, it can be an area of homology that is shared by at least two thirds of the parental polynucleotide sequences. Even more preferably a serviceable demarcation points is an area of homology that is shared by at least three fourths of the parental polynucleotide sequences, or, it can be shared by at almost all of the parental polynucleotide sequences. In one aspect, a demarcation point is an area of homology that is shared by all of the parental polynucleotide sequences.

In one aspect, a ligation reassembly process is performed exhaustively in order to generate an exhaustive library of progeny chimeric polynucleotides. In other words, all possible ordered combinations of the nucleic acid building blocks are represented in the set of finalized chimeric nucleic acid molecules. At the same time, in another aspect, the assembly order (i.e. the order of assembly of each building block in the 5′ to 3 sequence of each finalized chimeric nucleic acid) in each combination is by design (or non-stochastic) as described above. Because of the non-stochastic nature of this invention, the possibility of unwanted side products is greatly reduced.

In another aspect, the ligation reassembly method is performed systematically. For example, the method is performed in order to generate a systematically compartmentalized library of progeny molecules, with compartments that can be screened systematically, e.g. one by one. In other words this invention provides that, through the selective and judicious use of specific nucleic acid building blocks, coupled with the selective and judicious use of sequentially stepped assembly reactions, a design can be achieved where specific sets of progeny products are made in each of several reaction vessels. This allows a systematic examination and screening procedure to be performed. Thus, these methods allow a potentially very large number of progeny molecules to be examined systematically in smaller groups. Because of its ability to perform chimerizations in a manner that is highly flexible yet exhaustive and systematic as well, particularly when there is a low level of homology among the progenitor molecules, these methods provide for the generation of a library (or set) comprised of a large number of progeny molecules. Because of the non-stochastic nature of the instant ligation reassembly invention, the progeny molecules generated preferably comprise a library of finalized chimeric nucleic acid molecules having an overall assembly order that is chosen by design. The saturation mutagenesis and optimized directed evolution methods also can be used to generate different progeny molecular species. It is appreciated that the invention provides freedom of choice and control regarding the selection of demarcation points, the size and number of the nucleic acid building blocks, and the size and design of the couplings. It is appreciated, furthermore, that the requirement for intermolecular homology is highly relaxed for the operability of this invention. In fact, demarcation points can even be chosen in areas of little or no intermolecular homology. For example, because of codon wobble, i.e. the degeneracy of codons, nucleotide substitutions can be introduced into nucleic acid building blocks without altering the amino acid originally encoded in the corresponding progenitor template. Alternatively, a codon can be altered such that the coding for an originally amino acid is altered. This invention provides that such substitutions can be introduced into the nucleic acid building block in order to increase the incidence of intermolecular homologous demarcation points and thus to allow an increased number of couplings to be achieved among the building blocks, which in turn allows a greater number of progeny chimeric molecules to be generated.

In another aspect, the synthetic nature of the step in which the building blocks are generated allows the design and introduction of nucleotides (e.g., one or more nucleotides, which may be, for example, codons or introns or regulatory sequences) that can later be optionally removed in an in vitro process (e.g. by mutagenesis) or in an in vivo process (e.g. by utilizing the gene splicing ability of a host organism). It is appreciated that in many instances the introduction of these nucleotides may also be desirable for many other reasons in addition to the potential benefit of creating a serviceable demarcation point.

In one aspect, a nucleic acid building block is used to introduce an intron. Thus, functional introns are introduced into a man-made gene manufactured according to the methods described herein. The artificially introduced intron(s) can be functional in a host cells for gene splicing much in the way that naturally-occurring introns serve functionally in gene splicing.

Optimized Directed Evolution System

In practicing the methods of the invention a non-stochastic gene modification system termed “optimized directed evolution system” can be used to generate modified sequences encoding chimeric polypeptides of the invention with new or altered properties. Optimized directed evolution is directed to the use of repeated cycles of reductive reassortment, recombination and selection that allow for the directed molecular evolution of nucleic acids through recombination. Optimized directed evolution allows generation of a large population of evolved chimeric sequences, wherein the generated population is significantly enriched for sequences that have a predetermined number of crossover events.

A crossover event is a point in a chimeric sequence where a shift in sequence occurs from one parental variant to another parental variant. Such a point is normally at the juncture of where oligonucleotides from two parents are ligated together to form a single sequence. This method allows calculation of the correct concentrations of oligonucleotide sequences so that the final chimeric population of sequences is enriched for the chosen number of crossover events. This provides more control over choosing chimeric variants having a predetermined number of crossover events.

In addition, this method provides a convenient means for exploring a tremendous amount of the possible protein variant space in comparison to other systems. Previously, if one generated, for example, 10¹³ chimeric molecules during a reaction, it would be extremely difficult to test such a high number of chimeric variants for a particular activity. Moreover, a significant portion of the progeny population would have a very high number of crossover events which resulted in proteins that were less likely to have increased levels of a particular activity. By using these methods, the population of chimerics molecules can be enriched for those variants that have a particular number of crossover events. Thus, although one can still generate 10¹³ chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

One method for creating a chimeric progeny polynucleotide sequence is to create oligonucleotides corresponding to fragments or portions of each parental sequence. Each oligonucleotide preferably includes a unique region of overlap so that mixing the oligonucleotides together results in a new variant that has each oligonucleotide fragment assembled in the correct order. Additional information can also be found, e.g., in U.S. Ser. No. 09/332,835; U.S. Pat. No. 6,361,974. The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonucleotides from each parental variant in alternating order is very low. If each oligonucleotide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a ⅓ chance (assuming 3 parents) that an oligonucleotide from the same parental variant will ligate within the chimeric sequence and produce no crossover.

Accordingly, a probability density function (PDF) can be determined to predict the population of crossover events that are likely to occur during each step in a ligation reaction given a set number of parental variants, a number of oligonucleotides corresponding to each variant, and the concentrations of each variant during each step in the ligation reaction. The statistics and mathematics behind determining the PDF is described below. By utilizing these methods, one can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events. These methods are directed to the use of repeated cycles of reductive reassortment, recombination and selection that allow for the directed molecular evolution of a nucleic acid encoding a polypeptide through recombination. This system allows generation of a large population of evolved chimeric sequences, wherein the generated population is significantly enriched for sequences that have a predetermined number of crossover events. A crossover event is a point in a chimeric sequence where a shift in sequence occurs from one parental variant to another parental variant. Such a point is normally at the juncture of where oligonucleotides from two parents are ligated together to form a single sequence. The method allows calculation of the correct concentrations of oligonucleotide sequences so that the final chimeric population of sequences is enriched for the chosen number of crossover events. This provides more control over choosing chimeric variants having a predetermined number of crossover events.

In addition, these methods provide a convenient means for exploring a tremendous amount of the possible protein variant space in comparison to other systems. By using the methods described herein, the population of chimerics molecules can be enriched for those variants that have a particular number of crossover events. Thus, although one can still generate 10¹³ chimeric molecules during a reaction, each of the molecules chosen for further analysis most likely has, for example, only three crossover events. Because the resulting progeny population can be skewed to have a predetermined number of crossover events, the boundaries on the functional variety between the chimeric molecules is reduced. This provides a more manageable number of variables when calculating which oligonucleotide from the original parental polynucleotides might be responsible for affecting a particular trait.

In one aspect, the method creates a chimeric progeny polynucleotide sequence by creating oligonucleotides corresponding to fragments or portions of each parental sequence. Each oligonucleotide preferably includes a unique region of overlap so that mixing the oligonucleotides together results in a new variant that has each oligonucleotide fragment assembled in the correct order. See also U.S. Ser. No. 09/332,835.

The number of oligonucleotides generated for each parental variant bears a relationship to the total number of resulting crossovers in the chimeric molecule that is ultimately created. For example, three parental nucleotide sequence variants might be provided to undergo a ligation reaction in order to find a chimeric variant having, for example, greater activity at high temperature. As one example, a set of 50 oligonucleotide sequences can be generated corresponding to each portions of each parental variant. Accordingly, during the ligation reassembly process there could be up to 50 crossover events within each of the chimeric sequences. The probability that each of the generated chimeric polynucleotides will contain oligonucleotides from each parental variant in alternating order is very low. If each oligonucleotide fragment is present in the ligation reaction in the same molar quantity it is likely that in some positions oligonucleotides from the same parental polynucleotide will ligate next to one another and thus not result in a crossover event. If the concentration of each oligonucleotide from each parent is kept constant during any ligation step in this example, there is a ⅓ chance (assuming 3 parents) that an oligonucleotide from the same parental variant will ligate within the chimeric sequence and produce no crossover.

Accordingly, a probability density function (PDF) can be determined to predict the population of crossover events that are likely to occur during each step in a ligation reaction given a set number of parental variants, a number of oligonucleotides corresponding to each variant, and the concentrations of each variant during each step in the ligation reaction. The statistics and mathematics behind determining the PDF is described below. One can calculate such a probability density function, and thus enrich the chimeric progeny population for a predetermined number of crossover events resulting from a particular ligation reaction. Moreover, a target number of crossover events can be predetermined, and the system then programmed to calculate the starting quantities of each parental oligonucleotide during each step in the ligation reaction to result in a probability density function that centers on the predetermined number of crossover events.

Iterative Processes

In practicing the invention, these processes can be iteratively repeated. For example a nucleic acid (or, the nucleic acid) responsible for an altered phenotype of a chimeric polypeptide of the invention is identified, re-isolated, again modified, re-tested for activity using the methods of the invention. This process can be iteratively repeated until a desired phenotype is engineered. For example, an entire biochemical anabolic or catabolic pathway can be engineered into a cell, including proteolytic activity.

Similarly, if it is determined that a particular oligonucleotide has no affect at all on the desired trait, it can be removed as a variable by synthesizing larger parental oligonucleotides that include the sequence to be removed. Since incorporating the sequence within a larger sequence prevents any crossover events, there will no longer be any variation of this sequence in the progeny polynucleotides. This iterative practice of determining which oligonucleotides are most related to the desired trait, and which are unrelated, allows more efficient exploration all of the possible protein variants that might be provide a particular trait or activity.

Producing Sequence Variants

In practicing the methods of the invention nucleic acid variants can be generated using genetic engineering techniques such as site directed mutagenesis, random chemical mutagenesis, Exonuclease III deletion procedures, and standard cloning techniques. Alternatively, such variants, fragments, analogs, or derivatives may be created using chemical synthesis or modification procedures. Other methods of making variants are also familiar to those skilled in the art. These include procedures in which nucleic acid sequences obtained from natural isolates are modified to generate nucleic acids which encode polypeptides having characteristics which enhance their value in industrial or laboratory applications. In such procedures, a large number of variant sequences having one or more nucleotide differences with respect to the sequence obtained from the natural isolate are generated and characterized. These nucleotide differences can result in amino acid changes with respect to the polypeptides encoded by the nucleic acids from the natural isolates.

For example, variants may be created using error prone PCR. In error prone PCR, PCR is performed under conditions where the copying fidelity of the DNA polymerase is low, such that a high rate of point mutations is obtained along the entire length of the PCR product. Error prone PCR is described, e.g., in Leung, D. W., et al., Technique, 1:11-15, 1989) and Caldwell, R. C. & Joyce G. F., PCR Methods Applic., 2:28-33, 1992. Briefly, in such procedures, nucleic acids to be mutagenized are mixed with PCR primers, reaction buffer, MgCl₂, MnCl₂, Taq polymerase and an appropriate concentration of dNTPs for achieving a high rate of point mutation along the entire length of the PCR product. For example, the reaction may be performed using 20 fmoles of nucleic acid to be mutagenized, 30 pmole of each PCR primer, a reaction buffer comprising 50 mM KCl, 10 mM Tris HCl (pH 8.3) and 0.01% gelatin, 7 mM MgCl₂, 0.5 mM MnCl₂, 5 units of Taq polymerase, 0.2 mM dGTP, 0.2 mM dATP, 1 mM dCTP, and 1 mM dTTP. PCR may be performed for 30 cycles of 94° C. for 1 min, 45° C. for 1 min, and 72° C. for 1 min. However, it will be appreciated that these parameters may be varied as appropriate. The mutagenized nucleic acids are cloned into an appropriate vector and the activities of the polypeptides encoded by the mutagenized nucleic acids is evaluated.

Variants may also be created using oligonucleotide directed mutagenesis to generate site-specific mutations in any cloned DNA of interest. Oligonucleotide mutagenesis is described, e.g., in Reidhaar-Olson (1988) Science 241:53-57. Briefly, in such procedures a plurality of double stranded oligonucleotides bearing one or more mutations to be introduced into the cloned DNA are synthesized and inserted into the cloned DNA to be mutagenized. Clones containing the mutagenized DNA are recovered and the activities of the polypeptides they encode are assessed.

Another method for generating variants is assembly PCR. Assembly PCR involves the assembly of a PCR product from a mixture of small DNA fragments. A large number of different PCR reactions occur in parallel in the same vial, with the products of one reaction priming the products of another reaction. Assembly PCR is described in, e.g., U.S. Pat. No. 5,965,408.

Still another method of generating variants is sexual PCR mutagenesis. In sexual PCR mutagenesis, forced homologous recombination occurs between DNA molecules of different but highly related DNA sequence in vitro, as a result of random fragmentation of the DNA molecule based on sequence homology, followed by fixation of the crossover by primer extension in a PCR reaction. Sexual PCR mutagenesis is described, e.g., in Stemmer (1994) Proc. Natl. Acad. Sci. USA 91:10747-10751. Briefly, in such procedures a plurality of nucleic acids to be recombined are digested with DNase to generate fragments having an average size of 50-200 nucleotides. Fragments of the desired average size are purified and resuspended in a PCR mixture. PCR is conducted under conditions which facilitate recombination between the nucleic acid fragments. For example, PCR may be performed by resuspending the purified fragments at a concentration of 10-30 ng/:l in a solution of 0.2 mM of each dNTP, 2.2 mM MgCl₂, 50 mM KCL, 10 mM Tris HCl, pH 9.0, and 0.1% Triton X-100. 2.5 units of Taq polymerase per 100:1 of reaction mixture is added and PCR is performed using the following regime: 94° C. for 60 seconds, 94° C. for 30 seconds, 50-55° C. for 30 seconds, 72° C. for 30 seconds (30-45 times) and 72° C. for 5 minutes. However, it will be appreciated that these parameters may be varied as appropriate. In some aspects, oligonucleotides may be included in the PCR reactions. In other aspects, the Klenow fragment of DNA polymerase I may be used in a first set of PCR reactions and Taq polymerase may be used in a subsequent set of PCR reactions. Recombinant sequences are isolated and the activities of the polypeptides they encode are assessed.

Variants may also be created by in vivo mutagenesis. In some aspects, random mutations in a sequence of interest are generated by propagating the sequence of interest in a bacterial strain, such as an E. coli strain, which carries mutations in one or more of the DNA repair pathways. Such “mutator” strains have a higher random mutation rate than that of a wild-type parent. Propagating the DNA in one of these strains will eventually generate random mutations within the DNA. Mutator strains suitable for use for in vivo mutagenesis are described, e.g., in PCT Publication No. WO 91/16427.

Variants may also be generated using cassette mutagenesis. In cassette mutagenesis a small region of a double stranded DNA molecule is replaced with a synthetic oligonucleotide “cassette” that differs from the native sequence. The oligonucleotide often contains completely and/or partially randomized native sequence.

Recursive ensemble mutagenesis may also be used to generate variants. Recursive ensemble mutagenesis is an algorithm for protein engineering (protein mutagenesis) developed to produce diverse populations of phenotypically related mutants whose members differ in amino acid sequence. This method uses a feedback mechanism to control successive rounds of combinatorial cassette mutagenesis. Recursive ensemble mutagenesis is described, e.g., in Arkin (1992) Proc. Natl. Acad. Sci. USA 89:7811-7815.

In some aspects, variants are created using exponential ensemble mutagenesis. Exponential ensemble mutagenesis is a process for generating combinatorial libraries with a high percentage of unique and functional mutants, wherein small groups of residues are randomized in parallel to identify, at each altered position, amino acids which lead to functional proteins. Exponential ensemble mutagenesis is described, e.g., in Delegrave (1993) Biotechnology Res. 11:1548-1552. Random and site-directed mutagenesis are described, e.g., in Arnold (1993) Current Opinion in Biotechnology 4:450-455.

In some aspects, the variants are created using shuffling procedures wherein portions of a plurality of nucleic acids which encode distinct polypeptides are fused together to create chimeric nucleic acid sequences which encode chimeric polypeptides as described in, e.g., U.S. Pat. Nos. 5,965,408; 5,939,250.

Optimizing Codons to Achieve High Levels of Protein Expression in Host Cells

In one aspect of the invention, nucleic acids are mutated to modify codon usage. In one aspect, methods of the invention comprise modifying codons in a nucleic acid encoding a modified sequence encoding a chimeric polypeptide of the invention to increase or decrease its expression in a host cell, e.g., a bacterial, insect, mammalian, yeast or plant cell. The method can comprise identifying a “non-preferred” or a “less preferred” codon in protein-encoding nucleic acid and replacing one or more of these non-preferred or less preferred codons with a “preferred codon” encoding the same amino acid as the replaced codon and at least one non-preferred or less preferred codon in the nucleic acid has been replaced by a preferred codon encoding the same amino acid. A preferred codon is a codon over-represented in coding sequences in genes in the host cell and a non-preferred or less preferred codon is a codon under-represented in coding sequences in genes in the host cell.

Transgenic Non-Human Animals

The invention provides transgenic non-human animals comprising a nucleic acid, a polypeptide (e.g., Can A, NANODEX™ polypeptide), an expression cassette or vector or a transfected or transformed cell of the invention. The invention also provides methods of making and using these transgenic non-human animals.

The transgenic non-human animals can be, e.g., goats, rabbits, sheep, pigs, cows, rats and mice, comprising the nucleic acids of the invention. These animals can be used, e.g., to produce polypeptides of the invention in monomer, or polymer, form, or, as in vivo models to study Can A properties or activity, or, as models to screen for agents that change the Can A activity in vivo. The coding sequences for the polypeptides to be expressed in the transgenic non-human animals can be designed to be constitutive, or, under the control of tissue-specific, developmental-specific or inducible transcriptional regulatory factors. Transgenic non-human animals can be designed and generated using any method known in the art; see, e.g., U.S. Pat. Nos. 6,211,428; 6,187,992; 6,156,952; 6,118,044; 6,111,166; 6,107,541; 5,959,171; 5,922,854; 5,892,070; 5,880,327; 5,891,698; 5,639,940; 5,573,933; 5,387,742; 5,087,571, describing making and using transformed cells and eggs and transgenic mice, rats, rabbits, sheep, pigs and cows. See also, e.g., Pollock (1999) J. Immunol. Methods 231:147-157, describing the production of recombinant proteins in the milk of transgenic dairy animals; Baguisi (1999) Nat. Biotechnol. 17:456-461, demonstrating the production of transgenic goats. U.S. Pat. No. 6,211,428, describes making and using transgenic non-human mammals which express in their brains a nucleic acid construct comprising a DNA sequence. U.S. Pat. No. 5,387,742, describes injecting cloned recombinant or synthetic DNA sequences into fertilized mouse eggs, implanting the injected eggs in pseudo-pregnant females, and growing to term transgenic mice whose cells express proteins related to the pathology of Alzheimer's disease.

Transgenic Plants and Seeds

The invention provides transgenic plants and seeds comprising a nucleic acid, a polypeptide (e.g., Can A, NANODEX™ polypeptide), an expression cassette or vector or a transfected or transformed cell of the invention. The invention also provides plant products, e.g., oils, seeds, leaves, extracts and the like, comprising a nucleic acid and/or a polypeptide (e.g., Can A, NANODEX™ polypeptide) of the invention. The transgenic plant can be dicotyledonous (a dicot) or monocotyledonous (a monocot). The invention also provides methods of making and using these transgenic plants and seeds. The transgenic plant or plant cell expressing a polypeptide of the present invention may be constructed in accordance with any method known in the art. See, for example, U.S. Pat. No. 6,309,872.

Nucleic acids and expression constructs of the invention can be introduced into a plant cell by any means. For example, nucleic acids or expression constructs can be introduced into the genome of a desired plant host, or, the nucleic acids or expression constructs can be episomes. Introduction into the genome of a desired plant can be such that the host's Can A production is regulated by endogenous transcriptional or translational control elements. The invention also provides “knockout plants” where insertion of gene sequence by, e.g., homologous recombination, has disrupted the expression of the endogenous gene. Means to generate “knockout” plants are well-known in the art, see, e.g., Strepp (1998) Proc Natl. Acad. Sci. USA 95:4368-4373; Miao (1995) Plant J 7:359-365. See discussion on transgenic plants, below.

The nucleic acids of the invention can be used to confer desired traits on essentially any plant, e.g., on starch-producing plants, such as potato, wheat, rice, barley, and the like. Nucleic acids of the invention can be used to manipulate metabolic pathways of a plant in order to optimize or alter host's expression of Can A. The can change Can A properties, or activity, in a plant. Alternatively, Can A of the invention can be used in production of a transgenic plant to produce a compound not naturally produced by that plant. This can lower production costs or create a novel product.

In one aspect, the first step in production of a transgenic plant involves making an expression construct for expression in a plant cell. These techniques are well known in the art. They can include selecting and cloning a promoter, a coding sequence for facilitating efficient binding of ribosomes to mRNA and selecting the appropriate gene terminator sequences. One exemplary constitutive promoter is CaMV35S, from the cauliflower mosaic virus, which generally results in a high degree of expression in plants. Other promoters are more specific and respond to cues in the plant's internal or external environment. An exemplary light-inducible promoter is the promoter from the cab gene, encoding the major chlorophyll a/b binding protein.

In one aspect, the nucleic acid is modified to achieve greater expression in a plant cell. For example, a sequence of the invention is likely to have a higher percentage of A-T nucleotide pairs compared to that seen in a plant, some of which prefer G-C nucleotide pairs. Therefore, A-T nucleotides in the coding sequence can be substituted with G-C nucleotides without significantly changing the amino acid sequence to enhance production of the gene product in plant cells.

Selectable marker gene can be added to the gene construct in order to identify plant cells or tissues that have successfully integrated the transgene. This may be necessary because achieving incorporation and expression of genes in plant cells is a rare event, occurring in just a few percent of the targeted tissues or cells. Selectable marker genes encode proteins that provide resistance to agents that are normally toxic to plants, such as antibiotics or herbicides. Only plant cells that have integrated the selectable marker gene will survive when grown on a medium containing the appropriate antibiotic or herbicide. As for other inserted genes, marker genes also require promoter and termination sequences for proper function.

In one aspect, making transgenic plants or seeds comprises incorporating sequences of the invention and, optionally, marker genes into a target expression construct (e.g., a plasmid), along with positioning of the promoter and the terminator sequences. This can involve transferring the modified gene into the plant through a suitable method. For example, a construct may be introduced directly into the genomic DNA of the plant cell using techniques such as electroporation and microinjection of plant cell protoplasts, or the constructs can be introduced directly to plant tissue using ballistic methods, such as DNA particle bombardment. For example, see, e.g., Christou (1997) Plant Mol. Biol. 35:197-203; Pawlowski (1996) Mol. Biotechnol. 6:17-30; Klein (1987) Nature 327:70-73; Takumi (1997) Genes Genet. Syst. 72:63-69, discussing use of particle bombardment to introduce transgenes into wheat; and Adam (1997) supra, for use of particle bombardment to introduce YACs into plant cells. For example, Rinehart (1997) supra, used particle bombardment to generate transgenic cotton plants. Apparatus for accelerating particles is described U.S. Pat. No. 5,015,580; and, the commercially available BioRad (Biolistics) PDS-2000 particle acceleration instrument; see also, John, U.S. Pat. No. 5,608,148; and Ellis, U.S. Pat. No. 5,681,730, describing particle-mediated transformation of gymnosperms.

In one aspect, protoplasts can be immobilized and injected with a nucleic acids, e.g., an expression construct. Although plant regeneration from protoplasts is not easy with cereals, plant regeneration is possible in legumes using somatic embryogenesis from protoplast derived callus. Organized tissues can be transformed with naked DNA using gene gun technique, where DNA is coated on tungsten microprojectiles, shot 1/100th the size of cells, which carry the DNA deep into cells and organelles. Transformed tissue is then induced to regenerate, usually by somatic embryogenesis. This technique has been successful in several cereal species including maize and rice.

Nucleic acids, e.g., expression constructs, can also be introduced in to plant cells using recombinant viruses. Plant cells can be transformed using viral vectors, such as, e.g., tobacco mosaic virus derived vectors (Rouwendal (1997) Plant Mol. Biol. 33:989-999), see Porta (1996) “Use of viral replicons for the expression of genes in plants,” Mol. Biotechnol. 5:209-221.

Alternatively, nucleic acids, e.g., an expression construct, can be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. Agrobacterium tumefaciens-mediated transformation techniques, including disarming and use of binary vectors, are well described in the scientific literature. See, e.g., Horsch (1984) Science 233:496-498; Fraley (1983) Proc. Natl. Acad. Sci. USA 80:4803 (1983); Gene Transfer to Plants, Potrykus, ed. (Springer-Verlag, Berlin 1995). The DNA in an A. tumefaciens cell is contained in the bacterial chromosome as well as in another structure known as a Ti (tumor-inducing) plasmid. The Ti plasmid contains a stretch of DNA termed T-DNA (˜20 kb long) that is transferred to the plant cell in the infection process and a series of vir (virulence) genes that direct the infection process. A. tumefaciens can only infect a plant through wounds: when a plant root or stem is wounded it gives off certain chemical signals, in response to which, the vir genes of A. tumefaciens become activated and direct a series of events necessary for the transfer of the T-DNA from the Ti plasmid to the plant's chromosome. The T-DNA then enters the plant cell through the wound. One speculation is that the T-DNA waits until the plant DNA is being replicated or transcribed, then inserts itself into the exposed plant DNA. In order to use A. tumefaciens as a transgene vector, the tumor-inducing section of T-DNA have to be removed, while retaining the T-DNA border regions and the vir genes. The transgene is then inserted between the T-DNA border regions, where it is transferred to the plant cell and becomes integrated into the plant's chromosomes.

The invention provides for the transformation of monocotyledonous plants using the nucleic acids of the invention, including important cereals, see Hiei (1997) Plant Mol. Biol. 35:205-218. See also, e.g., Horsch, Science (1984) 233:496; Fraley (1983)

Proc. Natl. Acad. Sci. USA 80:4803; Thykjaer (1997) supra; Park (1996) Plant Mol. Biol. 32:1135-1148, discussing T-DNA integration into genomic DNA. See also D'Halluin, U.S. Pat. No. 5,712,135, describing a process for the stable integration of a DNA comprising a gene that is functional in a cell of a cereal, or other monocotyledonous plant.

In one aspect, the third step can involve selection and regeneration of whole plants capable of transmitting the incorporated target gene to the next generation. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee (1987) Ann. Rev. of Plant Phys. 38:467-486. To obtain whole plants from transgenic tissues such as immature embryos, they can be grown under controlled environmental conditions in a series of media containing nutrients and hormones, a process known as tissue culture. Once whole plants are generated and produce seed, evaluation of the progeny begins.

After the expression cassette is stably incorporated in transgenic plants, it can be introduced into other plants by sexual crossing. Any of a number of standard breeding techniques can be used, depending upon the species to be crossed. Since transgenic expression of the nucleic acids of the invention leads to phenotypic changes, plants comprising the recombinant nucleic acids of the invention can be sexually crossed with a second plant to obtain a final product. Thus, the seed of the invention can be derived from a cross between two transgenic plants of the invention, or a cross between a plant of the invention and another plant. The desired effects (e.g., expression of the polypeptides of the invention to produce a plant in which flowering behavior is altered) can be enhanced when both parental plants express the polypeptides (e.g., Can A) of the invention. The desired effects can be passed to future plant generations by standard propagation means.

The nucleic acids and polypeptides of the invention are expressed in or inserted in any plant or seed. Transgenic plants of the invention can be dicotyledonous or monocotyledonous. Examples of monocot transgenic plants of the invention are grasses, such as meadow grass (blue grass, Poa), forage grass such as festuca, lolium, temperate grass, such as Agrostis, and cereals, e.g., wheat, oats, rye, barley, rice, sorghum, and maize (corn). Examples of dicot transgenic plants of the invention are tobacco, legumes, such as lupins, potato, sugar beet, pea, bean and soybean, and cruciferous plants (family Brassicaceae), such as cauliflower, rape seed, and the closely related model organism Arabidopsis thaliana. Thus, the transgenic plants and seeds of the invention include a broad range of plants, including, but not limited to, species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannisetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, and Zea.

In alternative embodiments, the nucleic acids of the invention are expressed in plants which contain fiber cells, including, e.g., cotton, silk cotton tree (Kapok, Ceiba pentandra), desert willow, creosote bush, winterfat, balsa, ramie, kenaf, hemp, roselle, jute, sisal abaca and flax. In alternative embodiments, the transgenic plants of the invention can be members of the genus Gossypium, including members of any Gossypium species, such as G. arboreum; G. herbaceum, G. barbadense, and G. hirsutum.

The invention also provides transgenic plants, and methods for using them, for producing large amounts of the polypeptides (e.g., Can A, NANODEX™ polypeptide) of the invention. For example, see Palmgren (1997) Trends Genet. 13:348; Chong (1997) Transgenic Res. 6:289-296 (producing human milk protein beta-casein in transgenic potato plants using an auxin-inducible, bidirectional mannopine synthase (mas1′,2′) promoter with Agrobacterium tumefaciens-mediated leaf disc transformation methods).

Using known procedures, one of skill can screen for plants of the invention by detecting the increase or decrease of transgene mRNA or protein in transgenic plants. Means for detecting and quantitation of mRNAs or proteins are well known in the art.

### Methodologies and Devices

In practicing the invention, a variety of apparatus and methodologies can be used, e.g., using the chimeric monomers and polymers for chiral selection, to determine the efficiency of the chiral separation from a racemic mixture, as biosynthetic pathways, as selection scaffoldings, to screen for variant chimeric polypeptides, to determine the extent of nanotubule formation, and the like.

Capillary Arrays

Capillary arrays, such as the GIGAMATRIX™, Diversa Corporation, San Diego, Calif., can be used to practice the invention. Nucleic acids or polypeptides (the chimeric monomers and polymers of the invention) or other compositions (e.g., substrates or co-factors for using the nanotubule biosynthetic pathways of the invention, antibodies or other compounds for binding to chimeric monomers of the invention) can be immobilized to or applied to an array, including capillary arrays. Arrays can be used in the chiral selection methods of the invention. Capillary arrays can provide a system for holding and screening samples, monomers of the invention, chiral products selected by the methods of the invention, substrates and co-factors and products used in the biosynthetic pathway methods of the invention, and the like.

A sample apparatus can include a plurality of capillaries formed into an array of adjacent capillaries, wherein each capillary comprises at least one wall defining a lumen for retaining a sample. The apparatus can further include interstitial material disposed between adjacent capillaries in the array, and one or more reference indicia formed within of the interstitial material. A capillary for screening a sample, wherein the capillary is adapted for being bound in an array of capillaries, can include a first wall defining a lumen for retaining the sample, and a second wall formed of a filtering material, for filtering excitation energy provided to the lumen to excite the sample.

A polypeptide or other composition can be introduced into a first component into at least a portion of a capillary of a capillary array. Each capillary of the capillary array can comprise at least one wall defining a lumen for retaining the first component. An air bubble can be introduced into the capillary behind the first component. A second component can be introduced into the capillary, wherein the second component is separated from the first component by the air bubble. A sample of interest can be introduced as a first liquid labeled with a detectable particle into a capillary of a capillary array, wherein each capillary of the capillary array comprises at least one wall defining a lumen for retaining the first liquid and the detectable particle, and wherein the at least one wall is coated with a binding material for binding the detectable particle to the at least one wall. The method can further include removing the first liquid from the capillary tube, wherein the bound detectable particle is maintained within the capillary, and introducing a second liquid into the capillary tube.

The capillary array can include a plurality of individual capillaries comprising at least one outer wall defining a lumen. The outer wall of the capillary can be one or more walls fused together. Similarly, the wall can define a lumen that is cylindrical, square, hexagonal or any other geometric shape so long as the walls form a lumen for retention of a liquid or sample. The capillaries of the capillary array can be held together in close proximity to form a planar structure. The capillaries can be bound together, by being fused (e.g., where the capillaries are made of glass), glued, bonded, or clamped side-by-side. The capillary array can be formed of any number of individual capillaries, for example, a range from 100 to 4,000,000 capillaries. A capillary array can form a micro titer plate having about 100,000 or more individual capillaries bound together.

Arrays, or “Biochips”

In practicing the invention polypeptides of the invention can be immobilized to or applied to an array. Arrays can be used to practice the methods of the invention, e.g., chiral selection from a racemic mixture. Polypeptide arrays” can be used to simultaneously quantify or select for a plurality of proteins. The present invention can be practiced with any known “array,” also referred to as a “microarray” or “DNA array” or “nucleic acid array” or “polypeptide array” or “antibody array” or “biochip,” or variation thereof. Arrays are generically a plurality of “spots” or “target elements,” each target element comprising a defined amount of one or more biological molecules immobilized onto a defined area of a substrate surface for specific binding to a sample molecule. Any immobilization method can be used, e.g., immobilization upon an inert support such as diethylaminoethyl-cellulose, porous glass, chitin or cells. In practicing the methods of the invention, any known array and/or method of making and using arrays can be incorporated in whole or in part, or variations thereof, as described, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston (1998) Curr. Biol. 8:R171-R174; Schummer (1997) Biotechniques 23:1087-1092; Kern (1997) Biotechniques 23:120-124; Solinas-Toldo (1997) Genes, Chromosomes & Cancer 20:399-407; Bowtell (1999) Nature Genetics Supp. 2.1:25-32. See also published U.S. patent applications Nos. 20010018642; 20010019827; 20010016322; 20010014449; 20010014448; 20010012537; 20010008765.

### Antibodies and Manipulating the Immune Response

The cannulae chimeric proteins, e.g., as recombinant fusion proteins (chimeric monomers) of the invention can comprise cannulae polypeptides (e.g., CanA, CanB, CanC, CanD, CanE) to elicit an immune response, to initiate an immune response, to modulate an immune response, to suppress immune response, to monitor an immune response. Thus, the invention provides vaccines comprising monomers or polymers of the invention. Also provided are formulations and methods for administering compositions of the invention to elicit an immune response, to initiate an immune response, to modulate an immune response, to suppress immune response or to monitor an immune response.

The chimeric proteins of the invention can be used as the immunizing reagent alone or with other compositions, e.g., an adjuvant. The invention provides methods to make fusion monomers, which can be recombinant proteins, using cannulae polypeptides (e.g., CanA, CanB, CanC, CanD, CanE) that incorporate a one or a combination of antigenic epitopes and/or immunomodulatory domains (T-cell epitopes, B-cell epitopes, heat shock protein domains, immunogens, toleragens, enzymes, cytokines, carbohydrates, small molecules, etc.) to elicit an immune response, to initiate an immune response, to modulate an immune response, to suppress immune response or to monitor an immune response.

Chimeric monomers of the invention (comprising one or more of these heterologous domains, e.g., fusion-partner protein domains) can be assembled in varying ratios or amounts, e.g., on a polymer such as a nanotubule, where they can be arranged in a desired orientation, e.g., internally or externally. Chimeric monomers can be incubated under conditions that drive self-assembly of the monomers into polymer, e.g., nanotubule. In one aspect, a heteropolymer of the invention comprises one or more antigenic determinants and one or more immunomodulatory (e.g., immunostimulatory) domains. In alternative aspects, the heterologous domain is attached or fused to the N-terminus, a loop domain of CanA, and/or a C-terminus of a cannulae protein, e.g., CanA CanB etc. The heterologous domains (e.g., fusion partners) can be displayed on an interior surface, an external surface, or both, of an assembled tubular polymer. In one aspect, heterologous domains (e.g., fusion partners) are positioned on the exterior or interior of a nanotubule to manipulate the half-life of the heterologous domain. Surface-displayed heterologous domains are digested more rapidly by host proteases than interior-facing (less accessible, more shielded) domains.

The invention also provides polymers, e.g., nanotubes, presenting an arranged (including any engineered, pre-arranged display, e.g., a uniformly close-packed, or alternatively, loosely packed) array of chosen epitopes, including peptide, polypeptide or carbohydrate epitopes, for example, epitopes comprising immunogens, toleragens, etc. (the invention provides polymers of any desired three dimensional structure presenting a uniformly close-packed (or loose packed, if desired) array of chosen motifs, including a heterologous polypeptide or peptide, a carbohydrate, a small molecule, a nucleic acid or a lipid). This polymer of the invention can form a highly oriented three dimensional scaffold of amplified epitope density. Thus, in one aspect, this polymer can direct the proliferation and differentiation of progenitor cells. The polymer of the invention can be used in vitro, in vivo or in situ. These polymers of the invention can act as scaffold materials, e.g., as described herein, and in one aspect can provide a biodegradable and/or highly oriented, spirally symmetrical epitope display on an infinitely modifiable template structure. Alternative aspects provide customized modifications of the epitope sequence for a specific cell response.

The compositions and methods of the invention also can be practiced using antibodies. For example, the invention provides antibodies that can bind exclusively to polymers of the invention, or, chimeric proteins of the invention (e.g., and not to monomers). Antibodies also can be used in a biosynthetic pathway of the invention, or, an antibody that specifically binds to an enzyme, co-factor, substrate and the like for use in a biosynthetic pathway of the invention, or, an antibody that binds to a chiral selection protein or peptide used in the methods of the invention.

As discussed below, the chimeric polypeptides and/or the nanotubes of the invention can be used to generate “functionalized” filaments, fibers, threads and the like, which in turn, are used to generate novel “functionalized” textiles, fabrics, sheets, filters, coatings, pharmaceuticals, implants, “bio-adhesives”, and the like, of the invention. In one aspect, the filaments, fibers, threads and the like is “functionalized” with an antibody. Antibodies also can be used in immunoprecipitation, staining, immunoaffinity columns, and the like, to, e.g., purify chiral selection products or products of the biosynthetic pathways of the invention.

Methods of doing assays, e.g., ELISAs, with polyclonal and monoclonal antibodies are known to those of skill in the art and described in the scientific and patent literature, see, e.g., Coligan, CURRENT PROTOCOLS IN IMMUNOLOGY, Wiley/Greene, N.Y. (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler (1975) Nature 256:495; Harlow (1988) ANTIBODIES, A LABORATORY MANUAL, Cold Spring Harbor Publications, New York. Antibodies also can be generated in vitro, e.g., using recombinant antibody binding site expressing phage display libraries, in addition to the traditional in vivo methods using animals. See, e.g., Hoogenboom (1997) Trends Biotechnol. 15:62-70; Katz (1997) Annu. Rev. Biophys. Biomol. Struct. 26:27-45.

The ability of proteins in a biological sample to bind to the antibody may be determined using any of a variety of procedures familiar to those skilled in the art. For example, binding may be determined by labeling the antibody with a detectable label such as a fluorescent agent, an enzymatic label, or a radioisotope. Alternatively, binding of the antibody to the sample may be detected using a secondary antibody having such a detectable label thereon. Particular assays include ELISA assays, sandwich assays, radioimmunoassays, and Western Blots.

### Products of Manufacture

The invention provides products of manufacture comprising chimeric polypeptides of the invention, underivatized cannulae proteins, or a combination thereof. The monomers or polymers (nanotubules, bundles, filaments or sheets) of the invention, or cannulae proteins, can be incorporated into any material to make any product of manufacture, including fabrics, textiles, fibers, filters, detoxifying agent, coatings, sheets, adhesives, liquids, sprays, powders, aerosols, pharmaceuticals, tablets, pills, lotions and the like, in any manner using any process. For example, monomers or polymers (nanotubules, bundles, filaments or sheets) of the invention, or cannulae proteins, can be incorporated into a product of manufacture in an initial manufacturing process, adding on (e.g., application by liquid, powder, lotion or spray) after manufacture, or, by recombinant expression, e.g., in vitro or in vivo, such as expression of recombinant protein in a cell (e.g., a plant cell, such as a cotton plant fiber), as with transgenic plants, e.g., cotton, flax, corn, hemp, tobacco, and the like (see also, discussion regarding transgenic plants of the invention, above).

Textiles, Fabrics, and Flame Retardant, or Heat or Water Resistant Materials

Polypeptides, including the monomers and polymers, of the invention can also be used as flame (fire) retardant materials or to make a material heat resistant (or, more heat resistant). In one aspect, polypeptide polymers of the invention are used to imbue a heat resistance characteristic to a material, e.g., to make a material heat resistant, or, relatively more heat resistant. In one aspect, polypeptide polymers of the invention are used to make a material water resistant. In one aspect, polypeptide polymers of the invention (including products of manufacture of the invention) are fire (flame) retardant fabrics or textiles, electronic or medical devices, and the like, wherein the polymers are arrayed polymers units, which, in one aspect, are hollow, and the air-filled space act as an insulator or thermal retention mechanism.

The invention provides flame (fire) retardant, heat resistant and melt resistant compositions, e.g., products of manufacture (e.g., textiles, fabrics, fibers, medical devices, electronics), comprising a cannulae protein polymer (for example, nanotubes, bundles, filaments or sheets), e.g., comprising a cannulae protein polymer of the invention. In one aspect, the product of manufacture of the invention comprises a cannulae protein polymer (e.g., a NANODEX™ polymer), which can comprise a polypeptide of the invention, where the polymer forms a nanotube and provides a natural product amenable to a host of modification and optimization strategies. In another aspect, the product of manufacture of the invention comprising a cannulae protein polymer (e.g., a NANODEX™ polymer), which can comprise a polypeptide of the invention, is very thermally stable, and like other natural protein-based materials such as silk and wool, can ignite at significantly higher temperatures and exhibit self-extinguishing properties. In addition to the inherent flame-retardant characteristics of the product of manufacture of the invention comprising a cannulae protein polymer (e.g., a NANODEX™ polymer), which can comprise a polypeptide of the invention, comprise new chemical or protein groups for enhanced flame retardancy or new functionalities. The new chemical or protein groups can be added or assembled in any way, e.g., wherein a protein group comprises a heterologous polypeptide or peptide, where the invention provides chimeric polypeptides comprising at least a first domain comprising a cannulae polypeptide and a second domain comprising this heterologous polypeptide or peptide (new chemical or protein group). In one aspect, the new chemical or protein group is chemically linked (e.g., by linking groups) to a cannulae monomer before polymerization. In one aspect, the new chemical or protein group is chemically linked (e.g., by linking groups) to a cannulae polymer. In one aspect, the products of manufacture of the invention comprising a cannulae protein polymer (e.g., a NANODEX™ polymer), which can comprise a polypeptide of the invention, deliver new solutions to achieving fire (flame) retardancy in an economic, more effective and environmentally-friendly manner.

In aspect, the invention provides products of manufacture (e.g., textiles, fabrics, fibers) comprising cotton and a cannulae protein polymer (e.g., a NANODEX™ polymer), which can comprise a polypeptide of the invention. In one aspect, fibers comprising cotton and a cannulae protein polymer (e.g., a NANODEX™ polymer), which can comprise a polypeptide of the invention, are spun or woven into fabrics, clothing, textiles or other products, e.g., for electronic, medical devices, etc. In one aspect, cannulae protein polymers (e.g., a NANODEX™ polymers), which can comprise polypeptides of the invention, are spun with standard cotton or other polymer fibers. Thus, the invention provides flame retardant products of manufacture comprising cotton or synthetic polymers. In one aspect, the products of manufacture of the invention comprising cotton and a polymer of the invention will not ignite at the cotton-igniting 400° C. temperature, and will not support continued combustion by producing an afterglow. Hence, the invention provides multiple biochemical approaches to achieving fire/flame retardancy, and heat and melt resistance.

CanA-comprising nanotubules, as well as CanB-, CanC-, CanD, or CanE-comprising nanotubules of the invention and polypeptides (monomers) of the invention (e.g., NANODEX™ polypeptides) can exhibit remarkable heat stability, e.g. temperatures up to about 140° C. or 150° C., or more. The assembled polymer of the invention can be extremely stable, for example, withstanding 140° C. in 2% SDS detergent. In one aspect, polypeptide polymers of the invention are used as flame retardants incorporated into any material, e.g., fabrics (which can be designated NANOAVID™ textiles), fibers, adhesives, filters and the like. As noted above, Cannulae A, or CanA, is a heat-resistant protein capable of forming nanotubules. CanA-, CanB-, CanC-, CanD-, and/or CanE-comprising nanotubules can be assembled from monomeric subunits that self-assemble in the presence of divalent cation. In one aspect, monomers of the invention assemble into hollow rods with an outer diameter of approximately 25 nm and an inner diameter of approximately 20 nm. In one aspect, these exhibit molecular dimensions and an overall morphology not dissimilar to eukaryotic microtubules. CanA-, CanB-, CanC-, CanD-, and/or CanE-comprising monomers of the invention can be rapidly purified from bacterial extracts following heat treatment to remove the majority of the heat-labile host proteins. Following purification, CanA-, CanB-, CanC-, CanD-, and/or CanE-comprising monomers of the invention can self-assemble into nanotubules in the presence of e.g., calcium and magnesium at elevated temperature. The assembled nanotubule structure can turn arranged with a helical pitch. The CanA-, CanB-, CanC-, CanD-, and/or CanE-comprising nanotubules of the invention can be heat stable (up to 128° C.) and remain assembled in the presence of SDS or high concentrations of urea.

In one aspect, polypeptide polymers of the invention (e.g., nanotubules, bundles, filaments or sheets) comprise hydrophobic peptides or ionic moieties (including charged peptides), and/or incorporate alternate surface or interior side chains. These properties can be incorporated into a polypeptide polymers of the invention to make a material (e.g., a product of manufacture of the invention) water resistant (or relatively more water resistant), to make a material heat resistant (or relatively more heat resistant), to give a material insulating properties (or make it a relatively better insulator), or give the material better tensile or ductile strength.

Textiles and fibers comprising polypeptides of the invention can be advantageous over other materials, because, e.g., in some aspects, textiles or fibers of the invention can be made into longer fibers which spin more easily, can be relatively thin (to increase garment quality), and/or have increased tensile strength, which can be important during processing and wear for durability. Standardized tests which rate these qualities are easily performed and can be incorporated into any screening program involving assessment of a textile or fiber comprising polypeptides of the invention.

The invention provides processes that comprise, in addition to incorporation of a cannulae polymer (e.g., a polymer comprising a polypeptide of the invention) into a textile or fiber (e.g., cotton), additional treatments to make the textile or fiber more flame retardant. For example, a process of the invention can also comprise modification of the fibers, either in vivo or during processing to alter the intrinsic cellulose chemistry, addition of binding agents, or co-expression, in vivo, or co-processing with other ignition-resistant polymers. Thus, the invention provides processes for making flame (fire) retardant, heat resistant and melt resistant compositions (e.g., products of manufacture), including clothing, textiles, fibers, electronics and medical devices. In one aspect, products of manufacture of the invention (e.g., fabrics) made from these fibers ignite only at high temperature, burn slowly and rapidly self extinguish.

A process of the invention can also comprise transgenic expression of modifying enzymes which decorate the glucose substituents of the cellulose fibers. In one aspect, these modifying enzymes are attached to a polymer (e.g., a nanotube, bundle, filament or sheet) of the invention. This approach could use esterification, methylation, glycosylation or phosphorylation to modify cellulose as the fibers are being made in the cotton boll. Expression of the appropriate enzyme activity, specific for the desired reaction on the developing cellulose polymer would take advantage of existing cofactors to realize the desired modification.

A process of the invention can also comprise co-expression or transgenic expression of cellulose binding proteins. In one aspect, the second domain of a chimeric polypeptide of the invention comprises a cellulose binding protein. In one aspect, the cellulose binding proteins (e.g., cellulose binding domains) are attached to a polymer (e.g., nanotubes, bundles, filaments or sheets) of the invention. For example, the invention can incorporate use of polypeptide domains associated with endoglucanase enzymes which bind with very high affinity to cellulose chains. Expressing these at the time of fiber maturation can result in cotton fibers decorated with a proteinaceous coating. The affinity of the cellulose binding domains can help retain this quality throughout the garment lifetime. In one aspect, a chimeric polypeptide of the invention comprising a cellulose binding protein in a fabric or other material can inhibit flaming and glow by promoting microscopic charring. For cotton and other cellulose-based fibers, compositions comprising a chimeric polypeptide of the invention comprising a cellulose binding protein can provide a mechanism for binding. This approach can add high affinity cellulose-binding polypeptides, at chosen stoichiometries, at an amenable stage of fiber, tubule, filament, sheet or bundle processing. This can provide more control and can obviate any effect the cellulose binding proteins may have on fiber, filament, tubule, sheet or bundle development and early processing. Other approaches and manufacturing protocols may be incorporated in these processes of the invention for synthetic and other non-cellulose materials. In one aspect, cellulose binding domain (CBD) encoding nucleic acid sequences are be fused to cannulae (e.g., canA, canB, can C, canD, or cane) protein-encoding sequences to result in nanotubes, fibers, filaments, sheets or bundles with hundreds of high affinity binding sites for cellulose. In one aspect, the CBDs are displayed over the surface of the nanotube, and the linear tubes will orient in parallel to, and intercalate with, cellulose resulting in a proteinaceous component tightly associated within the cellulose fibril.

The density of CBDs on the surface of a fiber, tubule, filament, sheet or bundle can be controlled by adjusting the proportions of cannulae protein-CBD (which can be a chimeric protein of the invention), e.g., CanA-CBD, fusion partners in the pre-polymerization mixture. The high affinity of the CBDs for cellulose, the large number of interactions between CBD-comprising protein and cellulose, and the orientation of the fibers, tubules (e.g., nanotubules), filaments, sheets or bundles with cellulose fibrils will promote retention of the interaction throughout the lifetime of the product of manufacture (e.g., a garment, fabric, electronic or medical device).

In one aspect, the invention comprises use of well-described short polypeptide domains, e.g., about 20 to about 120 residues, or more, in length, associated with endoglucanase and xylanase enzymes which bind specifically to cellulose polymer. Exposure to high temperature (100° C.) in the presence of strongly denaturing reagents is required to remove these peptides from their cellulosic ligands, illustrating the high affinities between the cellulose binding domains (CBDs) and cellulose (Kₐ˜10⁻⁶M⁻¹ to amorphous and crystalline cellulose).

A process of the invention can also comprise transgenic expression of the Can A monomer (NANODEX™ monomer), 21 kDa in MW, a protein that self-assembles into a regular, tube-like, helically structured polymer with 28 monomeric subunits per turn, 25 nm outside diameter, 20 nm inside diameter, and 4-10 micron average length. The assembled polymer is very stable, withstanding 140° C. in 2% SDS. In one aspect, to leverage, adjust or engineer, a desired level of flame (heat) resistance for a protein, fiber, textile, etc., expression of the Can A gene product and/or a monomer or polymer of the invention is regulated. CanB, CanC, CanD and/or CanE monomers may also be used in the processes of the invention.

In one aspect, the tubular nature of a polymer of the invention, and the ability to control the interior and/or exterior chemistry of a polymer of the invention (e.g., a nanotube, filament, bundle or sheet) can impart additional characteristics to the material or product of manufacture of the invention (e.g., fabric, cloth, medical or electronic device), including thermal retention, water repellency, dyeability, antistatic and/or antibacterial properties, and/or sunlight resistance. In one aspect, the tube interior is filled with additional material, e.g., of a hydrophobic, hydrophilic, charged, liquid, solid, gas or metallic nature, either physically or by co-expression (e.g., of hydrophobic or similarly coexpressed and arrayed metal-binding proteins). In one aspect, this provides electrically or optically conductive, i.e., bioactive, heated or cooled, biosensing, or fluid repellant properties, to the material or product of manufacture of the invention (e.g., fabric, textile, cloth, medical or electronic device). The conducting fabric can be used as an “active” material for many applications, including environmental monitoring, bio- or chemical warfare detection, real-time physiological function sensing or other sensing applications.

In one aspect, biofunctional fibers, bundles, sheets, filaments, fabrics or textiles comprising polymers of the invention incorporating various protein chemistries are used in textiles, garments, cloth, medical or electronic devices, to provide functionalities and strength surpassing currently available natural fabrics or synthetic polymers. In one aspect, the inherent catalytic, electron transfer, light absorption and transmission properties of proteins can be utilized in stable, fused, oriented fibers. In one aspect, garment functionalities include enzyme-based detection and neutralization of chemical warfare or TIC agents or protein-based sensor monitoring of changes in immediate environment or in body physiology. In one aspect, protein chemistries are used to send and receive information by using light to activate protein-based signaling cascades, or to conduct current via redox biochemistry to transmit individual physiological or positional data. In one aspect, induced fluorescence or luminescence from molecularly oriented functional fabrics is used for “friend-or-foe” detection. In one aspect, functionalities are added to improve comfort or individual safety.

Functionalized Proteins and Products of Manufacture

The invention provides compositions, products of manufacture, and the like (e.g., NANOAVID™ electronics, textiles, fabrics, fibers, pharmaceuticals, liquids, powders, sprays, lotions, etc.), comprising monomers or polymers of the invention, or CanA CanB, CanC, CanD, CanE, or a combination thereof, comprising mixed populations of proteins (e.g., co-expressed proteins or co-binding on a protein polymer or nanotubular matrix), or, populations of small molecules, lipids, carbohydrates, nucleic acids and the like. In one aspect, these compositions of the invention provide a (complete or partial) natural product amenable to a host of modification and optimization strategies. In one aspect, the protein polymers comprising compositions and products of manufacture of the invention comprise densely packed amino acid polymers, which present an external surface of acidic and basic amino acid side chains, rendering them amenable to a range of chemical modifications.

In one aspect, the invention provides mixed populations of co-expressed proteins or co-bound proteins on nanotubular matrices or fibers comprising orientable, thermostable, functionalized, lightweight, insulating and/or conducting, fluid-repellant biomaterial, e.g., a biofabric. Compositions of the invention, e.g., NANOAVID™ textiles of the invention, can comprise mixed populations of coexpressed proteins co-binding on a protein copolymer nanotubular matrix. Compositions of the invention, e.g., NANOAVID™ textiles of the invention can be biofunctional materials, e.g., biofunctional fabrics. Compositions of the invention, e.g., NANOAVID™ textiles of the invention, can present protein chemistries that detect and neutralize toxic agents, respond to changes in the environment, and/or changes in body physiology. In addition, compositions, e.g., NANOAVID™ textiles, comprising monomers or polymers of the invention can comprise protein chemistries for sending and/or receiving information (for example, using light to activate a signaling cascade), or for conducting current. Functionalities may also be added to the compositions of the invention to improve comfort or individual safety. In alternative aspects, any attribute of a protein or enzyme may be expressed on a polymer surface. In one aspect, a fabric or textile of the invention can surpass conventional fabrics in both functional and mechanical characteristics based upon the advantages of its microscopic nanotubular structure, its ordered crystallinity, and its potential for protein-based functionalization and chemical modification.

The invention provides compositions, e.g., CanA, CanB, CanC, CanD, and/or CanE (NANOAVID™) biofibers or textiles, comprising monomers or polymers of the invention, that in some aspects can be conceptualized as a matrix formed by combining populations of fused polymer subunit monomers of the invention, or CanA, CanB, CanC, CanD or CanE (NANODEX™ monomers), or a combination thereof, to form functionalized nanotubular protein polymers. For example, fusion of biotin to some (or all) monomers results in presentation of biotin on the polymer surface (or, alternatively, in the polymer interior, e.g., the lumen of a nanotube). In one aspect, addition of avidin or streptavidin to the polymer population results in very strong inter-polymer associations and formation of arrayed filaments or fibers. In one aspect, a biofiber of the invention or other polymer of the invention can be used as a vehicle for catalytic, binding, emitting/absorbing, or other desired chemical functionalities. The desired chemical functionality can be added to a monomer population (e.g. CanA, CanB, CanC, CanD or CanE (NANOAVID™ monomers), or, monomers of the invention) before or after polymerization (including, e.g., self-assembly of monomers). The monomers and chemical functionalities can then be co-polymerized to create an active fiber. This “active fiber” of the invention can be woven (to itself, or, in conjunction with other fibers, such as cotton or synthetic fibers) to create electronics, medical devices, textiles, fabrics, clothing and the like, with varying capabilities, e.g., heat resistance, personal monitoring (e.g., with fabrics), signaling, decontamination or other desired characteristics (e.g., by filament or fiber spinning to create textiles or fabrics). The chemistry underlying the basic unit of fiber construction enables reassembly, or self-repair of the polymer or fabric.

Compositions and processes of the invention can also comprise co-expression and/or co-assembly of a monomer or polymer of the invention with a CanA, CanB, CanC, CanD or CanE monomer, or a combination thereof, resulting in the formation of NANODEX™-comprising tubular polymer, e.g., inside a cell or a fiber, e.g., a cotton fiber. For example, in one aspect, four to ten, or more, micron tubes are functionalized. In one aspect, they can bind to cellulose polymers at a chosen stoichiometry.

Processes of the invention can also comprise transgenic expression of genes or pathways expressing non-cellulosic biopolymers. In one aspect, non-cellulosic biopolymers (e.g., polyhydroxyalkanoates) are attached to a polymer (e.g., a nanotube) of the invention. Genes expressing polyhydroxyalkanoates, and other simple biopolymers have been described. In one aspect, the processes of the invention incorporate these pathways into a cell, a cotton cell, to result in the expression of a copolymer in the boll, which would then be co-processed with the cellulose fibers.

Processes of the invention can also comprise co-processing fibers with cellulose binding proteins. This aspect can add the high affinity cellulose-binding polypeptides, at chosen stoichiometries, at an amenable stage of fiber processing. This provides more control and obviates any effect the cellulose-binding polypeptide may have on fiber development and early processing.

Processes of the invention can also comprise esterification during processing. Controlled cellulose esterification with a chosen chemical agent can be performed biocatalytically at an optimal time during fiber processing. Appropriate choice of chemical alcohols and catalytic amounts of an esterase could render the fiber flame retardant. This approach can provide precise chemical control over cellulose decoration.

Processes of the invention can also comprise cellulose decoration during processing. Phosphorylation, methylation, glycosylation can be performed using catalytic amounts of appropriate kinase, methylase or glycosidase or glycosyl transferase. Performing these reactions during processing can provide control over decorating stoichiometry.

Processes of the invention can also comprise co-processing a plant fiber, e.g., a cotton fiber, with other biopolymers. In one aspect, the plant fiber comprises a monomer or a polymer of the invention. Polyalkanoates, or other microbially synthesized fibers can be co-spun with plant fibers (e.g., cotton fibers) at a predetermined fractional contribution.

The invention provides microarrays, filaments, sheets or bundles that can be used for bonding (e.g., “micro Velcro”) comprising monomers or polymers of the invention, or CanA, CanB, CanC, CanD or CanE (NANODEX™ monomers or polymers) or both. In one aspect, arrayed avid protein partners orient the polymers of the invention (e.g., tubes) into regular bundled or sheet-like structures and form a very stable, glue-like bond. The invention can be likened to a “micro Velcro” composition or product of manufacture which creates microarrays, filaments, sheets or bundles which can be spun or woven. In one aspect, the arrayed polymers units are hollow, and the air-filled space may act as an insulator.

The invention provides microarrays, filaments, sheets or bundles that can be used as bioactive, heated or cooled biosensing materials, or “conducting materials”, (compositions of manufacture) comprising monomers or polymers of the invention, or CanA, CanB, CanC, CanD or CanE (NANODEX™ monomers or polymers) or a combination thereof. The compositions of manufacture of the invention can have “spaced” interiors, e.g., nanotube or tube interiors, which may be filled with additional material, e.g., of a hydrophobic, liquid, gaseous or metallic nature, either physically or by coexpression (of hydrophobic or similarly coexpressed and arrayed metal-binding proteins) to provide electrically or optically conductive, i.e., bioactive, heated or cooled, biosensing, or fluid repellant properties, to the fabric. This “conducting material” or “conducting fabric” of the invention can be used as an “active” material for environmental monitoring, bio- or chemical warfare detection or detoxification, filters, real-time physiological function sensing or other sensing applications.

The functionalized nanotubes of the invention (comprising monomers or polymers of the invention, or CanA, CanB, CanC, CanD, or CanE (NANODEX™ monomers or polymers) or a combination thereof), combined with their simple structure and durability under extreme conditions renders them an excellent platform for use as robust, active materials. Because there are thousands of spirally-arrayed protein monomers (e.g., monomers of the invention, or CanA, CanB, CanC, CanD or CanE NANODEX™ monomers or polymers) or a combination thereof) in a single nanotube, and, since the polymerization proceeds stochastically and exothermically after mixing of monomers and addition of metal ions, by modulating the number of functionalized monomers of the invention in a mixture, or the identities of the functionalized monomers, fine control can be exerted over the ultimate functional display on the polymer surface or the tube interior. Coupling of any protein product and subsequent display of any catalytic, redox, light-absorbing, fluorescent, light or electron transmitting function can be envisioned in this aspect of the invention.

In one aspect, the invention provides chimeric polypeptides comprising at least a first domain comprising a cannulae polypeptide and a second domain comprising a heterologous polypeptide or peptide. Thus, the nanotubes of the invention serve as a foundation for generation of a “functionalized” filament or fiber, which in turn, are used to generate the novel “functionalized” textiles, fabrics, coatings, pharmaceuticals, “bio-adhesives”, and the like, of the invention. In one aspect, fibers of the invention are formed by separate coexpression of two populations of monomer, e.g., a multidomain (hetero-domained) monomer of the invention (for example, a monomer of the invention fused with one protein partner from a binding pair, e.g., an ultra-high affinity protein binding pair, such as biotin and avidin) and another monomer of the invention or a CanA, CanB, CanC, CanD and/or CanE monomer. In one aspect, biotin and avidin/streptavidin, or equivalent, are the ultra-high affinity protein binding pair (“avid pair”). The other monomer population can also be functionalized. Additional unfunctionalized subunits may be added to adjust the stoichiometries of subunits in the resultant polymer. In one aspect, each isolated population of fusion proteins (e.g., a multidomain (hetero-domained) monomer of the invention) is mixed and self-assembled into a polymer (e.g., a NANOAVID™ textile or fiber). In one aspect, the functionalized group, e.g., the fused biotin or enzyme moiety, is expressed on the surface of the polymer, or, in the interior of the polymer, or both, depending on their fusion position on the nucleic acid encoding the monomer of the invention (a multidomained, or hetero-domained, monomer of the invention).

In one aspect, the invention provides a process comprising mixing the resulting polymers with the ligand of the high affinity protein binder (e.g., mixing with a biotin binding protein, e.g., avidin). This should result in the binding of the high affinity protein binder ligand (e.g., biotin) to the binding protein (e.g., biotin binding protein, e.g., avidin) and result in an oriented array or filament of polymer. The resulting macrostructure will be based upon the high binding affinity of biotin to the biotin binding protein (e.g., biotin to avidin) and the spatial relationships of the biotin on the polymer surface. The arrayed superstructure might be likened to a “micro Velcro” with properties of self-repair. In one aspect, the filaments are spun into fibers, and/or subsequently woven into sheets or bundles, forming a robust fabric. This fabric will display characteristics imparted by the functionalization of the polymer of the invention.

The invention comprises use of any desired functionality. If desired, enzymes and genes can be screened for target chemistries and incorporated into the “functionalized” polymers of the invention. Optimization of enzyme phenotypes, i.e., productivity, selectivity and stability can be achieved by modifying nucleic acids encoding enzymes, or other desired functional groups (e.g., a binding protein) with standard methodologies, e.g., error-prone PCR, shuffling, oligonucleotide-directed mutagenesis, assembly PCR, sexual PCR mutagenesis, in vivo mutagenesis, cassette mutagenesis, recursive ensemble mutagenesis, exponential ensemble mutagenesis, site-specific mutagenesis, gene reassembly, Gene Site Saturation Mutagenesis™ (GSSM™), synthetic ligation reassembly (SLR) technologies (or a combination thereof).

The invention provides “biofunctional” products of manufacture, including “biofunctionalized” fabrics, textiles, sheeting, coverings, coatings, adhesives, filters and the like. In one aspect, a “biofunctional” products of manufacture of the invention is designed to detect and/or neutralize toxic agents (e.g., microorganisms (e.g., bacteria, viruses), spores (e.g., anthrax spores), chemical or biological toxins, poisons, poison gases, allergens, irritating particles, and the like). In one aspect, a “biofunctional” products of manufacture of the invention is designed to respond to changes in the environment or changes in body physiology. In one aspect, a “biofunctional” products of manufacture of the invention is designed to send and receive information; for example, using light to activate a signaling cascade or conduct current, e.g., in one aspect the invention provides a computer, data storage or related device, comprising a “biofunctional” product of manufacture of the invention. In one aspect, a “biofunctional” products of manufacture of the invention is designed with the appropriate functional groups to improve comfort or individual safety.

In one aspect, the invention provides products of manufacture for electronics and methods for making and using them; for example, the invention comprises coupling of target protein products with monomers or polymers of the invention and subsequent display of any hydrolytic protein, redox protein, light-absorbing composition, fluorescent composition, light or electron transmitting composition; semiconductors, and/or liquid crystals.

Self-Repairing, Reassembling “Active” Fabrics and Textiles

The invention also provides products of manufacture, e.g., fibers, fabrics, textiles, sheets, filters, adhesives, and the like, that are capable of “self-repair” through the ability of cannulae protein's ability to self-assemble. In one aspect, the invention provides multifunctional, self-repairing, “active” compositions, e.g., clothing, textiles or fabrics. The “biofibers” of the invention can form a matrix by combining mixed populations of protein subunit monomers (chimeric polypeptides of the invention, cannulae proteins, or a combination thereof) to form functionalized polymers of the invention (e.g., nanotubular protein polymers, bundles, filaments or sheets). Using a biofiber of the invention as a vehicle, catalytic, binding, emitting/absorbing, or other desired chemical functionalities can create an active fabric enabling personal monitoring, signaling, decontamination or other desired characteristics. The chemistry underlying the basic unit of fiber construction (cannulae protein ability to self-assemble) will enable reassembly, or self-repair of the fabric.

The products of manufacture of the invention can be used to increase the health, ability and potential of their users, e.g., individual warfighters in the military. For example, in one aspect, products of manufacture of the invention comprise wearable “smart” garments. In one aspect, the invention provides products of manufacture, e.g., fabrics or textiles, comprising “functionalized” polypeptides of the invention (including nanotubules) that comprise capabilities and properties enabling individual physical monitoring, external environmental sensing and transmitting, active decontamination (e.g., toxins, gases, poisons), self-repair, comfort and durability into a lightweight, wearable “smart” garments or devices (e.g., body armor). When such “smart” garments or devices are worn by individual warfighters, they will significantly improve survivability, morale and effectiveness of soldiers in combat.

In alternative aspects, the “biofunctional” textiles, fabrics, filters or devices of the invention can be multi-functional, for example, detect and neutralize toxic agents, respond to changes in the environment or changes in body physiology. In addition, protein chemistries can be incorporated into the compositions of the invention and used to send and receive information (for example, using light to activate a signaling cascade) or conduct current. Functionalities may also be added to improve comfort or individual safety.

In one aspect, the “biofunctional” product of manufacture of the invention (e.g., textiles, fabrics, fibers, filters, devices) of the invention are “functionalized” with detecting or detoxifying enzymes, e.g., a G-agent hydrolyzing enzyme sequence, such as orthophosphohydrolase, OPH, or paraoxonase (PON), or, esterases, acetylcholinesterases, butyrylcholinesterases, triesterases, cholinesterases, pseudocholinesterases, phosphor-triesterases, hydrolases, phosphohydrolases, organophosphate hydrases, and other organo-phosphorus and organosulfur hydrolyzing enzymes, peroxidases, chloroperoxidases, laccases, or a mixture thereof (e.g., a combination of organophosphate hydrolase and acetylcholinesterase or butyrylcholinesterase). These products of manufacture of the invention can be decontamination or detoxification devices. In one aspect, the detecting or detoxifying enzyme is the heterologous polypeptide or peptide of a chimeric polypeptide of the invention. Any enzyme capable of detoxifying organophosphorus compounds can be used. The G-agent hydrolyzing enzyme sequence (e.g., OPH) can be tested against G-agent surrogates paraoxon and diisopropyl-fluorophosphate. In this embodiment, the detoxifying/decontaminating product of manufacture of the invention, being functionalized with a hydrolyzing enzyme sequence, e.g., PON or OPH, can hydrolyze toxic metabolites of organophosphorus (OP) insecticides, pesticides and nerve agents. In one aspect, the detoxifying/decontaminating product of manufacture of the invention comprises metal oxide or hydroxide nanocrystals, e.g., MgO, SrO, BaO, CaO, TiO₂, ZrO₂, FeO, V₂O₃, V₂O₅, Mn₂O₃, Fe₂O₃, NiO, CuO, Al₂O₃, SiO₂, ZnO, Ag₂O, Mg(OH)₂, Ca(OH)₂, Al(OH)₃, Sr(OH)₂, Ba(OH)₂, Fe(OH)₃, Cu(OH)₃, Ni(OH)₂, Co(OH)₂, Zn(OH)₂, AgOH, and mixtures thereof, and the like; see, e.g., U.S. Pat. No. 6,653,519, which can be conjugated or attached to the product of manufacture via binding to a “functionalized” group on or part of (e.g., as a heterologous polypeptide or peptide of a chimeric polypeptide of the invention) a polymer or monomer of the invention.

In one aspect, the detoxifying/decontaminating product of manufacture of the invention comprises active filaments comprising nanotubes with fused detoxifying/decontaminating (e.g., toxin-degrading) enzymes, e.g., an organophosphohydrolase enzyme, and, in one aspect, biotinylated peptide sequences. In one aspect, the enzyme and peptide sequences are fused to a cannulae protein monomer at the gene level. In one aspect, the OPH gene is cloned from Flavobacterium.

Polymerization of chimeric monomers of the invention, or cannulae proteins, or mixtures thereof, to nanotubes can be initiated by divalent cation addition. When chimeric monomers of the invention, or cannulae proteins, or both, are “functionalized” with biotin, or equivalent, inter-nanotube associations in filaments can be initiated by avidin (or equivalent) addition. The time course and extent of polymerization of nanotubes from monomers and the formation of filaments from nanotubes can be followed by, e.g., light scattering spectrophotometry, e.g., using a Nepheloskan Ascent Type 750 nephelometer, or, by fluorescence polarization using, e.g., a Tecan Ultra spectrophotometer. Visual observation of nanotubes and nanofilaments can be made, e.g., using an Olympus microscope model AX70 with 100× optics. Temperature stability can be measured, e.g., by heat challenge and observed spectrophotometrically. Similar measurements can be used to observe pH and detergent stability.

In one aspect, the invention comprises fusing biotin affinity sequence to a cannulae protein, e.g., CanA, CanB, CanC, CanD and/or CanE, including fusions of a cannulae gene (e.g., CanA) with a biotin tag system (e.g., by Avidity Corp). The sequence can be fused to the N- and C-termini of the gene, or, can be inserted into the center of the cannulae gene (e.g., CanA, CanB, CanC, CanD and/or CanE) sequence. Constructs can be expressed in an in vivo biotinylation system, e.g., a system using a biotin ligase-containing E. coli host strain (AVB101 E. coli B). In one aspect, CanA, CanB, CanC, CanD and/or CanE monomers are polymerized and tested with added avidin for competence in fusion to filaments. CanA-, CanB-, CanC-, CanD- and/or CanE-biotin fusions can be admixed with non-fused CanA, CanB, CanC, CanD and/or CanE monomers in various stoichiometries to establish optimal biotin display densities. Nanotube polymerization can be followed by light scattering measured by nephelometry and/or by fluorescence polarization. Nanotubes will be characterized visually, i.e., length, branching, nanotubes/filament, etc., and for stability to temperature, detergent, pH, and the like.

In one aspect, the invention comprises fusing an OPH gene to a cannulae gene (e.g., CanA, CanB, CanC, CanD and/or CanE), and N- and C-terminal fusions of the Flavobacterium OPH gene are constructed to create a population of CanA-OPH monomers. In one aspect, this population is mixed with unfused cannulae (e.g., CanA, CanB, CanC, CanD and/or CanE) monomers to create nanotubes. These nanotubes can be characterized for fused OPH ability to hydrolyze the P—F bond of the G-agent surrogate substrates, paraoxon and diisopropylfluorophosphate, or equivalents using, e.g., LC-MS based assays.

In one aspect, the invention comprises co-polymerization of CanA-, CanB-, CanC-, CanD- and/or CanE-biotin, CanA-, CanB-, CanC-, CanD- and/or CanE-OPH and CanA, CanB, CanC, CanD and/or CanE subunits into nanotubes, bundles, filaments or sheets. Various conditions for the stoichiometric addition of CanA-, CanB-, CanC-, CanD- and/or CanE-biotin, CanA-, CanB-, CanC-, CanD- and/or CanE-OPH and CanA, CanB, CanC, CanD and/or CanE subunits are optimized for production of robust nanotubes, bundles, filaments or sheets with displayed biotin and OPH. Optimal stoichiometry is defined as the cannulae-biotin/cannulae-OPH/cannulae ratio giving best nanotube, bundle, filament or sheet length/stability characteristics, where the cannulae protein is, for example, CanA, CanB, CanC, CanD and/or CanE. Morphological as well as temperature and detergent stability characteristics is also monitored. Control over the number of biotin-avidin interactions via the control over stoichiometric mixing of biotinylated cannulae protein (e.g., CanA, CanB, CanC, CanD and/or CanE) subunits provides the ability to enhance or potentiate the number of inter-polymer interactions. In one aspect, the number of interactions provides a high degree of polymerization and low fiber density, making the fibers difficult to pull apart, either laterally or longitudinally.

In one aspect, the invention comprises forming active, bundles, filaments or sheets from nanotubes comprising monomers of the invention, cannulae (e.g., CanA, CanB, CanC, CanD and/or CanE) monomers, or a mixture thereof. Various conditions are established for the formation of active, bundles, filaments or sheets from the optimized cannulae-biotin/cannulae-OPH/cannulae nanotubes, where the cannulae is, for example, CanA, CanB, CanC, CanD and/or CanE. Filament formation can be followed spectrophotometrically and visually. Variables such as avidin/nanotube stoichiometries, biotin and/or enzyme densities, ionic strength and ambient pH can be assessed to optimize filament formation.

In one aspect, the invention comprises characterization of nanotubules, bundles, filaments or sheets of the invention (comprising monomers of the invention, cannulae (e.g., CanA, CanB, CanC, CanD and/or CanE) monomers, or a mixture thereof). In alternative embodiments, the invention comprises various populations of nanotubules, bundles, filaments or sheets of the invention differentiated by subunit stoichiometries and polymerization conditions. These can be characterized for morphology and stability as well as for other characteristics which render them fit for fabrication (e.g., into a product of manufacture) or spinning (e.g., into filaments or fibers), for example, hydrophilicity/hydrophobicity, linear versus branched orientation, inter-fiber interaction strength, filament tensile strength, etc. Catalytic competence for enzyme activity, e.g., paraoxon and/or diisopropylfluorophosphate hydrolysis, can be analyzed and optimized.

In one aspect, the invention comprises self-assembling, active filaments comprising fused nanotubular polymers (comprising monomers of the invention, cannulae (e.g., CanA CanB, CanC, CanD and/or CanE) monomers, or a mixture thereof) displaying OPH enzyme functionality. The filaments can be manufactured in a form (e.g., a kit) and amount ready for testing against G-agent surrogates, paraoxon and diisopropyl-fluorophosphate.

In one aspect, “biofunctional” products of manufacture of the invention comprise sponges or foams, including, in one aspect, products comprising monomers or polymers of the invention comprising functional groups comprising enzymatically active polypeptides. The sponge or foam may additionally contain activated carbon and an enzyme reactivation compound. See, e.g., U.S. Pat. Nos. 6,642,037; 6,541,230.

In one aspect, nanotubules, bundles, filaments or sheets of the invention of the invention are spun or woven into “biofibers”, which are then spun with standard cotton or other polymer fibers. In one aspect, the resulting thread or fiber comprises both nanotubules of the invention and cotton or other fiber, e.g., a synthetic polymer fiber (e.g., silk, polyester, nylon, rayon, KEVLAR®, NOMEX®, spider silk fiber, and the like). For example, in one aspect, the nanotubules, bundles, filaments or sheets of the invention of the invention are functionalized with enzymes, e.g., threads or fibers spun using these nanotubules are an “enzyme-enhanced” blended fiber or thread. An industrial spinner or a “spinerette” can be used to weave, or “co-spin”, the blended fiber.

In one aspect, cannulae proteins, including chimeric proteins of the invention (including polymers) are joined (e.g., co-spun) with amorphous fibers like wool (which alone are weak, easily elongated and poorly elastic while oriented), or with crystalline fibers like nylon, KEVLAR®, NOMEX® fibers (which are strong and rapidly recover from stretch). The supermolecular configuration of a polymer of the invention within a fabric or other material can be parallel and oriented with regard to the fiber's lengthwise axis. In this aspect, the resultant fiber can be crystalline and, relative to more amorphous structures, long and compact. At the molecular level, this translates to reduced tendency to tear and to increased tensile strength. At the macroscopic level, this translates into increased fabric or material flexibility, fatigue and damage resistance, strength and tenacity and the ability to self repair. This characteristic also obviates the need for stretching or drawing in textile processing. In one aspect, polymers of the invention, including fibers, threads, bundles, nanotubules comprising cannulae proteins, including chimeric proteins of the invention (e.g., NANODEX™ or NANOAVID™ polymers), are processed (e.g., co-spun or co-woven) with traditional or synthetic fibers (e.g., cotton, silk, polyester, nylon, rayon, KEVLAR®, NOMEX®, spider silk fiber, and the like) to provide new blends with desired characteristics, such as reduced flaming, heat resistance, insulating properties, etc. Additionally, in one aspect, new functionalities are added to the fabric blend, e.g., as enzyme-permeated or biotin-conjugated fibers for targeted applications, and other applications discussed herein.

In one aspect, the invention comprises methods for the attachment of monomers and/or polymers of the invention, cannulae proteins, or a mixture thereof, to cellulose in cotton or other natural or synthetic fibers. In one aspect, monomers and/or polymers of the invention, cannulae proteins, or both, are incorporated into cotton or other natural or synthetic fibers (e.g., linked or bound to cellulose in cotton) to generate fibers, fabrics, textiles, and the like, results in heat or flame resistant materials. For example, incorporation of polypeptides of the invention, cannulae proteins, or both, will prevent or inhibit ignition, glowing, melting or charring of the fabric or textile (e.g., cotton). In one aspect, nucleic acids encoding monomers and/or polymers of the invention, cannulae proteins, or both are expressed transgenically in a cell, e.g., a plant cell, such that they are expressed in the cell or resultant fiber, e.g., a cotton fiber (see discussion above on transgenic plants and non-human animals). This can result in a polymerized product of the invention co-expressed with the cellulose polymer. The expression and amount of the heterologous nucleic acid in the host cell or transgenic plant can be controlled by incorporation of appropriate transcriptional control elements, e.g., promoter elements. Alternatively, monomers and/or polymers of the invention, cannulae proteins, or both can be added the fiber or fabric (e.g., cotton) during processing or weaving. In one aspect, a nucleic acid expressing monomers and/or polymers of the invention, cannulae proteins, or both, can be altered to allow presentation of certain amino acid side-chains for functionalization, or to promote covalent or ionic association of the polymer with another moiety, e.g., a cellulose polymer. In one aspect, the invention is a replacement, or supplementation, for current chemical applications (e.g., halogenated hydrocarbons, such as polybrominated diphenyl ethers, or PBDE) to fabrics, textiles or fibers which can limit burning of cotton fabrics.

Voltage-Induced Processes for Making Polymers of the Invention

The invention provides processes for making polymers of the invention, including nanotubes, fibers, sheets, filaments and bundles, and products of manufacture (e.g., machines, medical devices), comprising cannulae proteins and/or chimeric polypeptides of the invention. In one aspect, the invention provides voltage-induced processes for making polymers of the invention. In one aspect, an applied voltage is used in the polymerization process to affect the intrinsic dipole moment of the polymer (e.g., a nanotubule), resulting in orientation of solutions of functionalized tubes, sheets, bundles, filaments, fibers and the like. The effect would be similar to that of a liquid crystal. The macroscopic dipole moment of the nanotubule will result from the summed contributions from spirally arrayed microscopic dipoles of monomers, e.g., chimeric polypeptides of the invention, or, CanA, CanB, CanC, CanD or CanE monomers, or, functionalized monomer subunits. In one aspect, the invention provides a means to display protein, catalytic or chemical functionalities on a nanoscale level in a spatially correlated fashion induced by applied voltage. In alternative aspects, the invention provides polymers of the invention made by this process, including polymers with optical, microelectric, photochemical and/or catalytic functionalities. These polymers can be used in sensors, computing applications, photo-bioelectronics, surface catalysts, multi-catalytic or other material or chemical applications.

Thus, the invention also provides products of manufacture such as sensors, computing or electronic devices, photo-bioelectronic devices, surface catalysts, multi-catalytic, medical devices or other materials comprising monomers or polymers of the invention and/or cannulae proteins, which, in one aspect, were made by use of an applied voltage in the polymerization process to affect the intrinsic dipole moment of the polymer (e.g., a nanotubule), resulting in orientation of solutions of functionalized tubes, sheets, bundles, filaments, fibers and the like.

Nanoarrays of the Invention

The invention provides spatially correlated, functional polymers of the invention, including nanotubes, fibers, sheets, filaments and bundles, comprising cannulae proteins and/or chimeric polypeptides of the invention, in the form of nanosheets or nanoarrays, or equivalent two-dimensional or three-dimensional structures. In one aspect, nanosheets or nanoarrays, or equivalent two-dimensional or three-dimensional structures of the invention have chemically patterned surfaces; see above discussions on functionalizing monomers and polymers of the invention, and, using applied voltage in the polymerization of monomers. In one aspect, the invention provides ordered arrays of cannulae proteins and/or chimeric polypeptides of the invention, wherein ordering is achieved by, e.g., attachment of polymers (e.g., nanotubules, fibers, filaments, etc.) to template surfaces using a protein-specific or a protein-directed chemical attachment moiety arranged on the surface, which results in binding of a subunit or many subunits (e.g., monomers) of the polymer, thus resulting in ordered arraying of the polymer. In one aspect, the nanosheets or nanoarrays, or equivalent two-dimensional or three-dimensional structures are horizontally or vertically arrayed on 2D or 3D surface templates. In one aspect, there is a high special correlation to yield functional arrays, sheets or films.

As discussed above, the polymers of the invention can be composed of regular spirally-arrayed protein monomers (e.g., chimeric proteins of the invention and/or cannulae protein monomers). The amino acid sidechains of the monomers can be chemically functionalized, or the sidechains substituted by other amino acids, to provide chemical functionality. Alternatively, nucleic acids encoding protein monomers can comprise coding sequence for enzymes, binding proteins and the like. In one aspect, this fusion protein is a chimeric protein of the invention, e.g., the second domain of a chimeric protein of the invention comprises an enzyme, binding protein and the like. In alternative aspects, the heterologous polypeptide(s) or peptide(s) of a chimeric protein of the invention is fused to the N-terminus, the C-terminus or both, and the heterologous polypeptide(s) or peptide(s) (the “functionality”) are displayed on the interior or the exterior of the polymer (e.g., polymerized tube).

In one aspect, the heterologous polypeptide(s) or peptide(s) (the “fused functionality”) are used as a means to attach monomers to a surface (e.g., a 2D or 3D surface template). Alternatively, amino acid chemistry or side chain chemistry exposed on the surface of the polymer is used as a means to attach monomers to a surface (e.g., a 2D or 3D surface template). Depending on the position of the chemical attachment or depending on the stoichiometry of the introduced attachment moieties, the polymer (e.g., a nanotube) can be oriented either vertically, or horizontally, or both (for a 3D template), with respect to the arraying substrate surface. In one aspect, the spiral symmetry of nanotubes and the regular display of cannulae monomers within the nanotube structure can direct the regular ordering of attached arrays.

Any number of chemistries can be used to attach protein subunits or amino acid sidechains displayed on subunits to a prepared surface. For example, using a flat surface, gold ion can be arrayed by sputtering or by atomic force microscopy. Any cysteine side chain can bind to the gold with high affinity. In one aspect, cysteine residues are engineered to be displayed on a cannulae protein surface, and this surface is designed to be on the outer surface of a polymer of the invention, e.g., a nanotube. Cysteine binding to the gold results in attachment of the polymer to the surface. In one aspect, cysteine-presenting monomers can be mixed in a controlled stoichiometry with non-cysteine presenting monomers. The gold-binding moieties can be presented in a way which can result in binding of polymers (e.g., nanotubes) in an ordered array with respect to a surface. In this aspect, if a single cannulae monomer comprising a “presented” cysteine were first bound to a surface (e.g., an array), and subsequently a second (additional) non-cysteine presenting monomer(s) were added, spontaneous polymerization would result in a vertically oriented-array (or, horizontally-oriented array, or both, depending on the design of the surface and the placing of the initial cysteine-comprising monomers). The amino acid sidechain chemistry of the non-cysteine presenting monomer(s) (subunits) can be adjusted to enhance packing of the arrayed polymers.

### Pharmaceutical, Medical Devices and Medical Uses

The invention provides pharmaceuticals, medical devices, biomimetic systems, surgical devices, artificial organs, prostheses, implants, and the like, comprising chimeric proteins and polymers (e.g., nanotubules, bundles, filaments or sheets) of the invention. Compositions of the invention can be used in any pharmaceutical, medical device, surgical device, dental device, artificial organ, prosthesis, implant, stent, catheter and the like, for example, as structural elements, coating, delivery vehicle (e.g., for antigens). Medical devices comprising a polypeptide of the invention, a cannulae protein, or both, include dental and orthopedic pins, screws, fixtures, implants and the like, plates, stents, stent sheaths, bypass grafts, catheters, cannulae, tissue scaffolds, wound care devices, dressings or implants, dental devices or implants, orthopedic or dental prostheses, and the like. The pharmaceuticals, medical devices, surgical devices, artificial organs, prostheses, implants of the invention can comprise “functionalized” filaments or fibers, e.g., the “functionalized” textiles, fabrics, sheets, filters, coatings, pharmaceuticals, “bio-adhesives” of the invention.

The invention also provides implants, cell transplant devices, tissue scaffolds, artificial joints, and the like, comprising a monomer or polymer of the invention. An exemplary medical device comprising a monomer or polymer of the invention comprises an implant or a tissue scaffold that can comprises a particular cell, e.g., a stem cell or other tissue rejuvenating cell, or a compound that can attract and/or bind a desired cell or a cell matrix compound or material. In one aspect an exemplary medical device comprising a monomer or polymer of the invention comprises a multicomponent polymer scaffold seeded with a tissue cell or a stem cell, e.g., a neural stem cell, vascular graft or skin graft material, a muscle stem cell, a tooth bud and the like. See, e.g., Teng (2002) Proc. Natl. Acad. Sci. USA 99:3024-3029 (Epub 2002 Feb. 26), describing a multicomponent polymer scaffold with neural stem cells used following traumatic spinal cord injury. Another exemplary device comprising a monomer or polymer of the invention is an ocular implant, e.g., a post-enucleation orbital implant, as described by Heimann (2005) Ophthal. Plast. Reconstr. Surg. 21:123-128. Another exemplary device comprising a monomer or polymer of the invention comprises dental implant, e.g., an alveolar ridge augmentation material, such as those prior to implant placement, e.g., as described by Busenlechner (2005) Clin. Oral Implants Res. 16:220-227; or tooth implant, e.g., as reviewed by Lemons (2004) J. Oral Implantol. 30:318-324. Another exemplary device comprising a monomer or polymer of the invention comprises prostheses or prostheses coatings, e.g., prostheses coatings used for primary total hip replacement, e.g., as reviewed by Lappalainen (2005) “Potential of coatings in total hip replacement” Clin. Orthop. Relat. Res. Jan; (430):72-79; Hilva (2005) Clin. Orthop. Relat. Res. Jan; (430):53-61.

Another exemplary device comprising a monomer or polymer of the invention comprises cell transplant device, e.g., as described by Langer and Vacanti (1993) Science 260:920-926; cell implant devices as described, e.g., by Wium (2005) “Managing chronic pain with encapsulated cell implants releasing catecholamines and endogenous opiods,” Front. Biosci. 10:367-378. Cell transplant and cell implant devices of the invention can be used to encapsulate and/or deliver cells, tissues and/or organs; for example: nerve cells (e.g., to treat dopamine deficiencies); skin (epidermal, dermal) cells (e.g., in wound or burn skin grafts); liver cells or tissues; kidney cells or tissue; pancreatic cells (e.g., to treat diabetes); to reconstruct tubular structures such as vascular elements (arteries, arterioles, veins as grafts or to repair), ureters, bladders, urethras, ducts and the like; bone, cartilage and/or muscle cells, including implants for bone or cartilage designed into shapes in whole or part configured by a polymer of the invention.

The polymers of the invention of the invention can be used in conjunction with other materials for tissue scaffold or medical devices, such as porous poly(DL-lactic-co-glycolic acid) (PLGA) or poly(L-lactic acid) (PLLA) foams as described by Lu (2000) Biomaterials 21:1837-1845; Lu (2000) Biomaterials 21:1595-605; Widmer (1998) Biomaterials 19:1945-1955.

In one aspect, where the chimeric proteins or polymers (e.g., nanotubules, bundles, filaments or sheets) of the invention are used as a delivery vehicle, e.g., for an antigen, epitope, toleragen, active biological agent (e.g., cytokine), cell matrix binding domain, carbohydrate, drug (e.g., small molecule), and the like, the active moiety (e.g., antigen, toleragen, active biological agent, drug) can be attached to a binding agent on a monomer (e.g., where the binding agent is the heterologous polypeptide or peptide of a chimeric polypeptide of the invention), or, where the heterologous polypeptide or peptide of a chimeric polypeptide of the invention comprises the antigen, toleragen, active biological agent or drug, e.g., as a recombinant protein. However, the “active moiety” can be attached in any way, including chemical linking, attraction by compositions having opposite charges, hydrophobic interactions, and the like.

In one aspect, the invention provides pharmaceutical compositions, e.g., liquids, solids (e.g., tablets, pills, implants), suspensions, lotions, aerosols or sprays, comprising chimeric proteins and/or nanotubules of the invention. The pharmaceutical composition can be a drug delivery device, where the drug delivery, or “targeting” moiety can be attached to a binding agent on a monomer (e.g., where the binding agent is the heterologous polypeptide or peptide of a chimeric polypeptide of the invention), or, where the heterologous polypeptide or peptide of a chimeric polypeptide of the invention comprises the drug delivery, or “targeting” moiety, e.g., as a recombinant protein. Alternatively, the invention provides pharmaceutical compositions for targeted drug or other substance (e.g., a nutrient, an active biological agent) delivery, where the delivery mechanism comprising chimeric proteins and/or nanotubules of the invention act by encapsulating the drug, nutrient, active biological agent, and the like.

In one aspect, “biofunctional” products of manufacture of the invention, e.g., as filters or biocatalytic devices, are designed to detoxify biological fluids, e.g., blood, for example, acting as filters or detoxifying agents in artificial livers or kidney (e.g., kidney dialysis filters) or other medical devices.

In one aspect, “biofunctional” products of manufacture of the invention comprise devices to contain or deliver or maintain whole cells, e.g., cultured cells, skin, tissues or artificial organs for implantation into an individual, e.g., implantation of pancreatic cells to treat diabetes (e.g., insulin deficient Type I diabetes), implantation of liver cells or kidney cells, or nerve cells, e.g., to treat Parkinson's disease. The whole cells containment device can be in any configuration, e.g., can be made using “functionalized” biofibers of the invention.

The invention provides tissue scaffolds or implant materials comprising monomers and polymers of the invention (e.g., a tubule or nanotubule, bundle, ball, fiber, filament, thread, or sheet). The tissue scaffold can comprise cells or tissues, e.g., graft material, stem cells, tissue culture cells, cadaver cells and the like. In one aspect, the invention provides a polymer scaffold with neural stem cells for repairing a spinal cord injury. In another aspect, the invention provides tissue scaffolds, e.g., an exemplary scaffold comprising a vascular graft. This exemplary tissue scaffold can have graft material comprising tissue or cells from smooth muscle, endothelial muscle and/or stem cells. Artery, vein or endothelial cells can be attached/bound to the tissue scaffold by binding to a chimeric polypeptides of the invention, which form as polymers comprising the tissue scaffold.

The invention also provides tissue engineering devices, such as biomimetic systems, comprising monomers and polymers of the invention for culturing or making tissues, e.g., growing or sustaining liver tissue or growing autologous blood vessels, e.g., small-caliber arteries, for example, as described by Niklason (1999) Science 284:489-493. For example, in one aspect, collagen fibers are bound to chimeric proteins of the invention comprising extracellular matrix or cellular matrix and/or collagen-binding domains. The chimeric proteins of the invention can be formed as a biomimetic tubule tissue scaffold for the growth of the blood vessel, e.g., small-caliber arteries.

Exemplary tissue engineering devices include devices for growing or sustaining liver tissue, e.g., artificial liver devices for purifying biological fluids, e.g., as described by U.S. Pat. Nos. 6,858,146; 6,379,710; 6,294,380; 5,976,870. Method for culturing liver cells is known, see, e.g., U.S. Pat. Nos. 6,727,066; 5,942,436. The chimeric proteins of the invention can comprise liver cell binding domains, or, domains that can bind to liver extracellular matrix proteins.

Exemplary tissue engineering devices also include devices for bone healing or bone or cartilage re-growth and/or regeneration, see, e.g., U.S. Pat. No. 6,743,232

The monomers and polymers of the invention (e.g., a tubule or nanotubule, bundle, ball, fiber, filament, thread, or sheet) can be used in the fabrication of any implant material, e.g., a biodegradable implant materials, such as the degradable thermoplastic polymers that can change shape after an increase in temperature (“shape-memory” polymers), as described e.g., by Lendlein (2002) Science 296:1673). In one aspect, the biodegradable implant material of the invention comprises covalently cross-linked polymer networks. In one aspect, macrodiols having different thermal characteristics are synthesized through ring-opening polymerization of cyclic diesters or lactones.

Pharmaceutical Formulations

The invention provides pharmaceutical compositions, e.g., as drug delivery devices or as vaccines or immunomodulatory compositions, comprising a chimeric protein of the invention and a pharmaceutically acceptable excipient. The invention provides parenteral formulations comprising a chimeric protein of the invention. The invention provides enteral formulations comprising a chimeric protein of the invention. The invention provides methods for administering a composition of the invention, e.g., as drug delivery device, vaccine or an immunomodulatory composition, comprising providing a pharmaceutical composition comprising a chimeric protein of the invention; and administering an effective amount of the pharmaceutical composition to a subject in need thereof. The pharmaceutical compositions used in the methods of the invention can be administered by any means known in the art, e.g., parenterally, topically, orally, or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration, the immune responsiveness on the part of a patient population (e.g., when used as a vaccine or an immunomodulatory composition) and the like. Details on techniques for formulation and administration are well described in the scientific and patent literature, see, e.g., the latest edition of Remington's Pharmaceutical Sciences, Maack Publishing Co, Easton Pa. (“Remington's”).

In alternative embodiments of the compositions and methods of the invention, pharmaceutical compounds can be formulated for and delivered transdermally, by a topical route, formulated as applicator sticks, solutions, suspensions, emulsions, gels, creams, ointments, pastes, jellies, paints, powders, and aerosols.

In the methods of the invention, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:857-863 (1995); or, as microspheres for oral administration, see, e.g., Eyles (1997) J. Pharm. Pharmacol. 49:669-674.

The compositions and formulations of the invention can be delivered by the use of liposomes. By using liposomes, particularly where the liposome surface carries ligands specific for target cells, or are otherwise preferentially directed to a specific organ, one can focus the delivery of the active agent into target cells in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708.

### Silicatein—(Silica Protein) Comprising Monomers or Polymers

In one aspect, products of manufacture of the invention are “biomimetics”, for example, in one aspect, a polymer of the invention is used as a “biomimetic”. In one aspect, silicatein (silica protein) enzyme is fused to a cannulae protein, e.g., as a recombinant protein, for polycondensation of silicon alkoxides, dioxane, oligo-oxane or polyoxane products. In one aspect, a silicatein-comprising monomers or polymers of the invention are used in material sciences, electronic or optical applications. In one aspect, the invention provides polymers (e.g., nanotubes) comprising chimeric polypeptides comprising at least a first domain comprising a cannulae polypeptide and at least a second domain comprising a heterologous polypeptide or peptide having silicatein activity. In one aspect, the nanotubes are spirally arrayed, e.g., the silicatein is spirally displayed on the exterior (or interior, or both) of a nanotube, fibril or the like comprising a silicatein-comprising chimeric polypeptide of the invention.

In one aspect, silicatein-comprising monomers or polymers of the invention are used to catalyze the formation of C—Si bonds. In alternative aspects, silicatein-comprising monomers or polymers of the invention are used to catalyze the formation of C—Te (tellurium) bonds, C—Se (selenium) bonds, and/or C—Ge (germanium) bonds. These silicatein-comprising monomers or polymers of the invention are used to catalyze and spatially direct the polycondensation of silicon alkoxides, metal alkoxides, and their organic conjugates to make silica, polysiloxanes, polymetallo-oxanes, and mixed poly(silicon/metalklo)oxane materials under environmentally benign conditions, see, e.g., U.S. Pat. No. 6,670,438. In one aspect, these polymers of the invention are used for the formation of geometrically arrayed silicate scaffolds, e.g., in material sciences, electronic or optical applications, or medical device or prosthetic applications. In one aspect, the invention provides a process using spatially arrayed silicatein-comprising monomers or polymers of the invention wherein silicon alkoxides are added to generate by polycondensation the formation of spatially oriented silicates. The final organization of these spatially oriented silicates can be determined by the original orientation of the catalytic moieties on the polymer, e.g., nanopolymer, such as a nanotube or nanofiber. The resulting structures have material properties of strength, conductivity and/or optical transmission imparted by the silicate chemistry and the ordered spiral symmetry. Accordingly, in alternative aspects, the invention provides electronic devices (e.g., computers, CD-ROMs, transistors, circuits, semiconductors, liquid crystals), optical devices (e.g., optical transmission devices), any device with light or electron transmitting function, light-absorbing devices, fluorescent devices, medical devices and the like comprising spatially oriented silicates made by a polymer of the invention, or, comprising a polypeptide of the invention.

In one aspect, the heterologous polypeptide or peptide having silicatein activity comprises all or parts or, or is derived from a diatom or a marine sponge, e.g., a Porifera, e.g., Suberites domuncula or Tethya aurantia, see, e.g., Krasko (2000) Eur. J. Biochem. 267(15):4878-4887, U.S. Pat. No. 6,670,438.

### Other Uses

In one aspect, “biofunctional” products of manufacture of the invention, e.g., as filters, are designed to detoxify soil, air or water, e.g., to degrade organic chemicals, e.g., pesticides, or other pollutants, in soil or water, or, to detoxify (e.g., remove) bacterial or spores (e.g., anthrax spores) from water, air or soil. In one aspect, “biofunctional” products of manufacture of the invention, e.g., as filters, are designed to filter toxins, poisons, spores or allergens from air or water.

In one aspect, the polypeptides of the invention are used in plant growth or plant tissue or cell implant devices, e.g., as grafts, in cell cultures, such as with plant protoplasts, cell growth matrices (e.g., as described by U.S. Pat. No. 6,385,903; 6,779,300) and the like.

### Kits

The invention provides kits comprising materials for practicing the invention, including monomers and polymers (e.g., nanotubules, bundles, filaments or sheets), of the invention. The kits can comprise solutions for assembling the nanotubules of the invention. For example, the solutions can comprise various salts, as described herein. The kits can comprise “biofunctional” products of manufacture of the invention, including “biofunctionalized” fabrics, textiles, sheeting, coverings, coatings, adhesives, filters, pharmaceuticals, and the like. The kits also can contain instructional material teaching the methodologies and uses of the invention, as described herein.

### EXAMPLES

### Example 1

### Isolating Recombinant Proteins from E. coli

The following example describes an exemplary assay to isolate recombinant “cannulae” or “can” proteins from E. coli.

All exemplary assays in this example used:

Low salt buffer: 80 mM NaCl, 50 mM Tris/HCl (pH 7.5), 9% glycerol

High salt buffer: 1.2 M NaCl, 50 mM Tris/HCl (pH 7.5), 9% glycerol

Bicinchonic Acid Test (BCA): The test was conducted according to the manufacturer's guide (Sigma, Deisenhofen). To this end, aliquots of protein samples (CanA, B, C) and of known BSA dilutions were mixed with 50 times the volume of a fresh BCA/CuSO₄ (50:1) solution, incubated at 60° C. for 30 min. and measured in the spectrometer at 562 nm after cooling to RT. The protein concentrations were measured with the BSA calibration line.

a) CanA and CanB

One gram of recombinant E. coli with a particular sequence such as CanA or CanB expressed was absorbed in 4 ml low salt buffer. Cell lysis was conducted with a French press (2× at 20,000 psi, American Instrument Co., Silver Spring, USA). After pelletizing the cell fragments (Eppendorf centrifuge, 13,000 rpm, 5 min., RT), the protein solution was incubated at 80° C. for 20 min. Then the denatured proteins were removed by centrifugation (as above). The supernatant was passed at 1 ml/min through a Q Sepharose column (1×12 cm=9.4 ml, Pharmacia, Freiburg). The eluent containing CanA or CanB was collected. The collected eluant was treated with leupeptin (1 μg/μl) and concentrated by a factor of 3-4 (based on the volume) in 4-8 hours in the MACROSEP™ centrifuge concentrators (Pall Filtron, Dreieich) with an exclusion limit of 5 kDA. After determining the protein concentration with the BCA test, the purified protein was shock frozen in liquid nitrogen in 100-200 μl aliquots and stored at −80° C. In each working step, a sample was taken and analyzed on an SDS polyacrylamide gel.

b) CanC

The first step of isolating CanC is same as that of CanA and CanB (see example 21.a). However, during the second step, CanC was retained on the Q sepharose. After flushing the column with low salt buffer, CanC was eluted from the column with a salt gradient (80-750 mM, in 60 ml) and collected by fractionation (1 ml each). Following analysis of the individual fractions on an SDS polyacrylamide gel, the CanC-containing fractions were combined and dialyzed against the low salt buffer at 4° C. overnight. Finally the protein solution was eluded at 1 ml/min through a 1 ml RESOURCEQ™ column (Pharmacia, Freiburg). Then a salt gradient (80-750 mM, in 60 ml) was applied and 0.5 ml fractions were collected. After analysis of the same on an SDS polyacrylamide gel, the CanC-containing fractions were combined again and dialyzed against low salt buffer overnight. Following addition of leupeptin (1 μg/μl), the solution was concentrated by a factor of 7 (based on the volume) in 6 hours in the MICROSEP™ centrifuge concentrators (Pall Filtron, Dreieich) with an exclusion limit of 5 kDa.

### Example 2

### Production of a CanA Polymer

The following example describes an exemplary protocol to produce a CanA polymer, including a chimeric polypeptide or a nanotubule of the invention.

a) 300L Fermentor Culture of Recombinant E. Coli.

A 300 L culture of recombinant E. coli BL21 (DE3) harboring expression plasmid pEX-CAN-A (produced by attaching sequence substantially identical to SEQ ID NO. 1 to a vector pET17b using a procedure described in Example 20) was grown in a HTE-Fermentor (Bioengineering, Wald, Switzerland) at 37° C. under aeration (165 L air/min.) and stirring (400 rpm) with a doubling time of about 40 min. At an O.D. (600 nm) of 0.80, production of Can A protein was induced by addition of 30 grams of IPTG. Cells were harvested 3 hours after the induction and after being cooled down to 4° C. Cell yield: 1,610 grams (wet weight).

b) Production of the Polymer.

i. French Press: 250 g frozen cell mass of recombinant E. coli (stored at −60° C.) were suspended in 600 ml buffer (Tris-HCL 50 mM, pH 7.5, containing 80 mM NaCl and 9% (v/v) glycerol). Final volume: 900 ml. Cells were broken down by a French Press (Aminco; 1×20,000 PSI). The viscosity of the solution was lowered by shearing the DNA using an Ultraturrax blender and by adding additional 400 ml buffer.

ii. Centrifugation: Particles were removed by centrifugation (Sorvall SS34 rotor; 19,000 rpm, 15 min.) and a clear supernatant (called “crude extract”) was obtained.

iii. Heat Precipitation: To precipitate the heat-sensitive protein, the crude extract was heated to 100° C. for 1 min. For example, the crude extract (1,200 ml) was pumped through a 75 cm long plastic hose (inner diameter, 5 mm; 4.75 ml/min) immersed in a 100° C. hot water-glycerol-bath (water: glycerol=1:1). The outlet end of the plastic hose was passed through an ice bath to cool down the solution in the hose before solution was finally collected using an Erlenmeyer flask.

iv. Centrifugation: The heat-treated crude extract was centrifuged for 25 min. at 9,000 rpm in Sorvall rotor GSA. The clear supernatant was collected.

v. Ammonium sulfate Precipitation: To the clear supernatant (840 ml), a 100% saturated ammonium sulfate solution (452 ml) was added at 4° C. (final ammonium sulfate concentration: 35% saturation). After 2 hours at 4° C., the precipitate was collected by centrifugation (1 hour; 13,000 rpm; Sowall rotor GSA). The precipitate was then solubilized in a buffer solution (final volume 171 ml; 12,35 mg protein/ml; 2,112 mg total protein) to form a protein solution. Finally, the protein solution was dialyzed by Rapid Dialysis against another buffer solution until its conductivity was the same as that of the buffer (3 hours).

vi. Polymerization: The dialyzed protein solution was diluted by addition of buffer to a final protein concentration of 6.5 mg/ml (final volume 325 ml). Then, under shaking in a 1L Erlenmeyer flask at 100° C. (in a water bath), the diluted protein solution was rapidly heated to 80° C. and then immediately transferred into a 500 ml screw-capped storage bottle. The storage bottle contained 3.32 ml (21.58 mg protein) of “Polymer Primers” (the “Polymer Primers” had been prepared before by 4 times French Press-shearing of a prefabricated Polymer suspension). Then, CaCl and MgCl (each at 20 mM final concentration) were added to the mixture and the closed bottle was stored in an 60° C. water bath. After addition of these salts, the solution became immediately turbid, indicating rapid polymerization of the protein units. After 10 min polymerization, the formed Polymer fibers were sheared by ultraturraxing the solution for 20 seconds in order to create additional polymer primers to speed up polymerization. Traces of silicone antifoam may be added before the ultraturraxing to reduce foaming. Typically, after 10 min. polymerization at 80° C., Polymer or polymer fibers could be observed under an electron microscope. After 1 to 2 hours of polymerization, protein polymers could be completely removed from the solution by centrifugation (15 min., 20,000 rpm, Sorvall rotor SS34), indicating complete polymerization.

Yield of polymer: 2.1 grams (protein) from 250 grams (wet weight) of E. coli (about 1 g Polymer (dry weight)/119 g E. coli).

vii. Storage: Wet: At 4° C. in a buffer containing 10 mM Na-Azide. Dry: Freeze-drying the polymer after the polymer being washed with an 1/10 diluted buffer followed by centrifugation.

### Example 3

### Preparation of Lipid Coated Drug Delivery Complexes

The following example describes an exemplary protocol to prepare lipid coated drug delivery complexes of the invention, e.g., pharmaceutical compositions comprising CanA, e.g., the chimeric polypeptides or nanotubules of the invention.

To a solution containing 3 mg/ml monomeric protein units (e.g. Can A: 182 amino acids: MW=19,830 daltons, having a sequence of SEQ ID NO. 2), a desired amount of drug molecules, and a sufficient amount of electrically neutral lipids, millimolar calcium and magnesium cations are added to form a mixture. The mixture is kept at ambient condition for a sufficient amount time until liposomes form. Thereafter, gel filtration chromatography is carried out on the mixture and the liposomes contained in the mixture are size fractionated. The desired fractions of the liposomes are then heated to 50° C. in the presence of millimolar amounts of calcium and magnesium cations to initiate the polymerization of the monomeric polypeptide units within each liposome. The polymerization results in the extreme deformation of the liposomes and produces sealed lipid tubules containing the drug molecules.

### Example 3

### Preparation of Nanotubules

The following example describes an exemplary protocol to prepare nanotubules of the invention.

Through genetic isolation techniques the inventors isolated cannulae-producing genes and cloned them using E. coli. canA, canB, and canC genes were isolated and artificially grown, and reproduced. These cannulae protein subunits are the monomers which undergo polymerization. In order to see the monomer under a microscope, Green Fluorescent Protein (GFP) were added to the A, B, and C terminal ends. Under some conditions the GFP proteins which were added acted to stabilize the polymers (e.g., the nanotubules) and allowed visualization of the polymerization reaction of cannulae forming monomer.

The fluorescent protein also was fused to the monomer to generate a fluorescent nanotube. These canA-GFP and GFP-canA fusion proteins could stabilize the protein for assembly to form a polymer, without the use of canB or canC. The effects of varying salt conditions for polymerization of these new proteins were unknown prior to these experiments.

The GFP protein was initially isolated from the Aequorea victoriaisa reporter molecule and fluoresces green when exposed to ultraviolet light. The stability of the GFP protein is species independent and its frequent ability to fuse to other proteins without inhibiting any original function allowed it to be freely attached to the canA protein. Recent research in GFP proteins has been able to produce mutant GFP proteins that are viewable under the whole visible spectrum, resulting in live action footage between living cells, and viewable fluorescence in light other than ultraviolet light. The GFP protein is also highly chemical and thermal stable, allowing experiments to be executed at temperatures at 80° C. and higher, due to its very compact structure. Due to the chemical stability and species independency of the GFP protein, the protein canA with the GFP on the carboxy-terminal end was able to be clearly viewed under the microscope without any interference with the original properties of the canA protein. However, without the addition of the GFP protein, the isolated canA protein did not efficiently polymerize due to instability of the protein, as it did in its native environment directly from the organism. Despite this finding, the properties and applications of the canA protein were unchanged from its native behavior by the addition of the GFP protein. This allowed the collection of decipherable data by use of the confocal microscope and viewing the green fluorescence from the fused GFP protein. In the GFP protein, the aromatic system of the chromophore determines the wavelength of fluorescence, i.e., the color of the fluorescence.

Due to the natural ability of the Pyrodictium abyssi organism to polymerize in the hydrothermal vents, a similar environment was generated to reproduce or enhance this reaction in the laboratory (the invention comprises methods for polymerizing chimeric proteins and nanotubules using such similar environments). In order to investigate this possibility, it was necessary to test the effects of different salts found in the deep sea vent environment on polymerization. One of a variety of choices of chemical salt catalysts, including copper sulfate, manganese sulfate, zinc sulfate, iron sulfate, magnesium sulfate, and calcium sulfate, lithium sulfate, and cobalt sulfate were also added as catalysts. Independent of the salt used, the polymerization reaction was almost instantaneous upon the addition of the appropriate chemical catalyst, although there were varying effects on the structure and length of the polymer being formed.

The initial monomer generated was a bright fluorescent green prior to the inclusion of chemical additives. After the polymerization reaction of the cannulae proteins, a cloudy precipitate formed that signified polymerization between the cannulae protein monomers. The reaction material was centrifuged to separate the polymerization product from excess reactants and was then examined under an Olympus Confocal microscope. Through the use of an argon (Ar) laser and reverse objective lenses, the fluorescent proteins were examined and photographed for study and comparison.

Capable of using over ten different laser types, the FluoView™500™ Confocal Microscope blocks all other light from entering the viewed specimen and gives a clear image of both fluorescence and nonfluorescent forms of the sample. In the case of the experiments described herein, the argon (Ar) laser was used to excite the GFP molecules of a fluorescent polymer of the invention on the slide in order to emit green light photons. The photons were viewed and captured on film to record the length of the individual strands of the polymers and also measure the dimensions of the strands. In a confocal microscope the scanning and image capture are both acquired through the objective lens which is under the specimen. The confocal microscope provides both a clear and detailed image of the polymers. However, due to the depth of the sample there can be difficulty in deciphering the exact individual strands of polymer. Since it is not possible to three dimensionally rotate the photograph once taken, there can be difficulty confirming whether what was examined is a single strand of polymerized protein, or several polymers stacked on top each other, forming the appearance of a single multi-folded chain. However, adjusting the PMT, Offset, and Gain on the microscope settings, accurate and readable data on the newly polymerized monomer was obtained.

The results of these experiments demonstrated that the methods of the invention comprising use of “deep sea salts” or equivalents are very effective in synthesizing nanotubules. Before the “deep sea salt initiation” processes of the invention, previous experiments had used magnesium chloride and calcium chloride. With the use of these salts with the wild type monomer polymerization would sometimes take days to occur. After examination with the use of the confocal microscope with argon (Ar) laser scanning, minimal sized polymer chains were observed far from the desired longer interweaving protein chains. Similarly, these salts were poor for forming polymers with the GFP canA protein. However, the processes of the invention (i.e., adding seawater salts, seawater solutions as described herein, or equivalent) were effective in generating protein chains and nanotubules. With salts such as manganese sulfate, the polymer chains were more then triple their original length. Thus, the processes of the invention are used to generate bionanotubes and compositions comprising them.

In some experiments the addition of copper sulfate not only did the monomer not polymerize, the addition of copper sulfate salt also stopped the GFP fluorescence. This was particularly unexpected. The GFP protein is generally considered very chemically stable and unaffected by salts. This result suggested that it was not necessarily just the fluorescence of the GFP protein that was inhibited, but the entire protein was disassembled. Supporting evidence for this was that not only were the biological nanotubes not viewable with green fluorescence, they were nonexistent upon translucent light inspection when the experiment was performed with a copper sulfate salt initiator. The entire reaction was stopped seeming to indicate a degradation of unpolymerized GFP fusion protein. The slide under the confocal microscope appeared to be blank, however, when the light was switched to translucent light, the monomer was viewable, but no polymer strands existed.

With this observation, a new hypothesis was developed. It was theorized, that since the organism was found in an environment where all these salts existed together, that better polymerization would occur if the positive salts were mixed together without the inhibitory salts, such as copper sulfate that is found in these deep ocean sites. After examining these results under the same conditions, and under a confocal microscope, using both Argon (Ar) and translucent light, it was found that with the addition of this mixture, these salts had varying results in different parts of the viewed microscope slide. This suggests that the mixture of the salts was not evenly distributed throughout the sample. In the mixed salt experiment slide, the results of the polymerization yielded at best only slightly larger polymers than their corresponding single salt experiments. Interestingly, copper sulfate was not inhibitory in these mixed salt experiments. Due to these results it was theorized that if all of the salts, including those that have negative gain, were mixed together to mimic the condition that these organisms thrive in then favorable results might be found due to reproducing the concentration balance found with the organism under original conditions. Thus, in one aspect, the invention provides processes comprising use of a solution comprising salts mixed together which are, in one aspect, the same or similar to the growth microenvironment of the organisms that naturally synthesize nanotubules comprising CanA, such as Pyrodictium abyssi.

A number of embodiments of the invention have been described. Nevertheless, it will be understood that various modifications may be made without departing from the spirit and scope of the invention. Accordingly, other embodiments are within the scope of the following claims.

1: A composition comprising (a) at least a first domain comprising a cannulae polypeptide and at least one additional domain comprising a non-cannulae polypeptide or peptide, a carbohydrate, a small molecule, a nucleic acid or a lipid;
(b) the composition of (a), wherein the non-cannulae polypeptide or peptide is inserted at the amino terminal end, the carboxy terminal end or internal to the cannulae polypeptide;
(c) the composition of (a) or (b), wherein the cannulae polypeptide comprises a protein having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity to SEQ ID NO:2, SEQ ID NO:4, SEQ ID NO:6, SEQ ID NO:8, SEQ ID NO:10 or SEQ ID NO:12;
(d) the composition of any of (a) to (c), wherein the cannulae polypeptide is capable of assembling into a polymer;
(e) the composition of any of (a) to (d), wherein the cannulae polypeptide is a recombinant or synthetic polypeptide, or the at least one additional domain comprises a polypeptide or peptide and the cannulae polypeptide and the polypeptide or peptide of the additional domain is a recombinant or synthetic polypeptide;
(f) the composition of any of (a) to (e), wherein the polymer acts as a biosynthetic pathway or a selection scaffolding;
(g) the composition of any of (a) to (f), wherein the composition is capable of acting as a chiral selector;
(h) the composition of any of (a) to (g), wherein the cannulae polypeptide comprises a protein having sequence as set forth in SEQ ID NO:2 SEQ ID NO:4, SEQ ID NO:6, SEQ ID NO:8, SEQ ID NO:10 or SEQ ID NO:12;
(i) the composition of any of (a) to (h), wherein the cannulae polypeptide comprises a FtsZ domain;
(j) the composition of any of (d) to (i), wherein the cannulae polypeptide is capable of assembling into a structure having an interior space;
(k) the composition of (j), wherein the structure having an interior space comprises a tubule or a nanotubule;
(l) the composition of (j), wherein the at least one additional domain is exposed into the inner lumen of the tubule or nanotubule;
(m) the composition of any of (d) to (i), wherein the at least one additional domain is expressed on the exterior of the tubule or nanotubule;
(n) the composition of any of (a) to (f), wherein the at least one additional domain comprises a chiral selection motif;
(o) the composition of any of (a) to (f), wherein the at least one additional domain comprises a receptor, a binding protein or a ligand;
(p) the composition of (o), wherein the binding protein comprises biotin;
(q) the composition of any of (a) to (p), wherein the non-cannulae polypeptide or peptide, or the at least one additional domain, comprises an enzyme;
(r) the composition of any of (a) to (f), wherein the non-cannulae polypeptide or Peptide, or the at least one additional domain, comprises an enzyme active site;
(s) the composition of any of (a) to (f), wherein the non-cannulae polypeptide or peptide, or the at least one additional domain, comprises an antigen or an antigen binding site;
(t) the composition of any of (a) to (f), wherein the non-cannulae polypeptide or peptide, or the at least one additional domain, comprises a green fluorescent protein, an alpha-galactosidase or a chloramphenicol acetyltransferase;
(u) the composition of any of (a) to (f), wherein the non-cannulae polypeptide or peptide, or the at least one additional domain, comprises a recombinant or synthetic protein;
(v) the composition of any of (a) to (u), wherein at least one subsequence of the cannulae polypeptide has been removed;
(w) the composition of (v), wherein the non-cannulae polypeptide is inserted into the cannulae polypeptide at the site the subsequence was removed;
(x) the composition of (w), wherein the cannulae polypeptide is a CanA polypeptide and the removed subsequence is a 14 residue motif consisting of residue 123 to residue 136 of SEQ ID NO:2 (PDKTGYTNTSIWVP), or, a 17 residue motif located at amino acid residue 123 to residue 139 of SEQ ID NO:2 (PDKTGYTNTSIWVPGEP);
(y) the composition of (v), wherein the non-cannulae polypeptide is inserted into the CanA polypeptide at the site a subsequence is removed; or
(z) the composition of (v), wherein the non-cannulae polypeptide is a 14 or a 17 residue motif inserted into the CanA polypeptide to replace a removed 14 or a 17 residue motif.

2-25. (canceled)

26: An immobilized composition comprising the composition of claim 1.

27: A tubule or nanotubule, bundle, ball, fiber, filament or sheet comprising
(a) a plurality of the compositions of claim 1;
(b) the tubule or nanotubule, bundle, ball, fiber, filament or sheet of (a), wherein the non-cannulae polypeptide comprises an enzyme or an enzyme co-factor;
(c) the tubule or nanotubule, bundle, ball, fiber, filament or sheet of (b), wherein the tubule or nanotubule, bundle, ball, fiber, filament or sheet comprises a plurality of different enzymes;
(d) the tubule or nanotubule, bundle, ball, fiber, filament or sheet of (c), wherein the plurality of enzymes comprises a biosynthetic pathway;
(e) the tubule or nanotubule, bundle, ball, fiber, filament or sheet of (c), wherein the plurality of enzymes are arranged along the length of the tubule or nanotubule, bundle, ball, fiber, filament or sheet in the same order as they act in the biosynthetic pathway;
(f) the tubule or nanotubule, bundle, ball, fiber, filament or sheet of any of (a) to (e), wherein the non-cannulae polypeptide comprises a chiral selection motif;
(g) the tubule or nanotubule, bundle, ball, fiber, filament or sheet of any of (a) to (f), wherein the non-cannulae polypeptide comprises a protein binding domain or small molecule binding domain; or,
(h) the tubule or nanotubule, bundle, ball, fiber, filament or sheet of (g), wherein the protein binding domain comprises a biotin.

28-34. (canceled)

35: A nucleic acid comprising a sequence encoding the composition of claim 1, wherein the at least one additional domain comprises a polypeptide or peptide.

36: An expression cassette or vector comprising the nucleic acid of claim 35.

37: A cell comprising
(a) the nucleic acid of claim 35, or the expression cassette or vector of claim 36; or
(b) the cell of (a), wherein the cell is a bacterial cell, a plant cell, a yeast cell, a fungal cell, an insect cell or a mammalian cell.

38. (canceled)

39: A transgenic non-human animal comprising the nucleic acid of claim 35, or the expression cassette or vector of claim 36.

40: A plant or a seed comprising the nucleic acid of claim 35 or the composition chimeric polypeptide of claim 1, or the expression cassette or vector of claim 36.

41: A method for the chiral selection of a specie of a racemic mixture, comprising:
(a) providing a composition of claim 6;
(b) providing a racemic mixture; and,
(c) contacting the racemic mixture with the composition under conditions wherein only one enantiomer of the composition binds to the composition; thereby selecting a single chiral specie of the racemic mixture.

42: A method for the chiral selection of a specie of a racemic mixture, comprising:
(a) providing the tubule or nanotubule, bundle, ball, fiber, filament or sheet of claim 27;
(b) providing the racemic mixture; and,
(c) contacting the racemic mixture with the tubule or nanotubule, bundle, ball, fiber, filament or sheet under conditions wherein only one enantiomer of the composition binds to the tubule or nanotubule, bundle, ball, fiber, filament or sheet; thereby selecting a single chiral specie of the racemic mixture.

43: A method for enzymatic biosynthesis of a composition, comprising:
(A) (a) providing the tubule or nanotubule, bundle, ball, fiber, filament or sheet comprising a plurality of enzymes comprising of biosynthetic pathway of claim 27;
(b) providing a substrate for at least one enzyme; and,
(c) contacting the tubule or nanotubule, bundle, ball, fiber, filament or sheet with the substrate under conditions wherein the enzymes of the biosynthetic pathway catalyze the synthesis of the compositions
(B) the method of claim (A), wherein the enzymes are expressed in the inner lumen of the tubule or nanotubule, bundle, ball, fiber, filament or sheet; or
(C) the method of claim (A), wherein the enzymes are expressed on the exterior of the tubule or nanotubule, bundle, ball, fiber, filament or sheet.

44-45. (canceled)

46: A cell comprising
(a) the composition of claim 1 or the tubule or a tubule or nanotubule, bundle, ball, fiber, filament or sheet of claim 27; or
(b) the cell of (a), wherein the cell is a bacterial cell, a plant cell, a yeast cell, a fungal cell, an insect cell or a mammalian cell.

47. (canceled)

48: A transgenic non-human animal comprising the chimeric protein of claim 1 or the tubule or a nanotubule of claim 27.

49: A plant or a seed comprising the chimeric protein of claim 1 or the tubule or nanotubule, bundle, ball, fiber, filament or sheet of claim 27.

50: A fiber comprising the tubule or nanotubule, bundle, ball, fiber, filament or sheet of claim 27.

51: A fabric or textile comprising the tubule or nanotubule, bundle, ball, fiber, filament or sheet of claim 27.

52: A fabric, textile, sheet or covering comprising a fiber or a thread comprising the tubule or nanotubule, bundle, ball, fiber, filament or sheet of claim 27, wherein the tubule or nanotubule, bundle, ball, fiber, filament or sheet is woven into a fabric, textile, sheet or covering.

53: A product of manufacture comprising
(a) the composition of claim 1 or the tubule or nanotubule, bundle, ball, fiber, filament or sheet of claim 27, a non-derivatized cannulae protein, or a combination thereof;
(b) the product of manufacture of (a), comprising a computer, a transistor or a circuit comprising the chimeric protein;
(c) the product of manufacture of (a) or (b), comprising a sheeting, a covering, a coating or an adhesive comprising the chimeric protein; or
(c) the product of manufacture of any of (a) to (c), comprising a flame retardant or heat resistant device comprising a sheeting, a covering, a coating or an adhesive comprising the chimeric protein.

54-56. (canceled)

57: A medical device or an implant comprising the chimeric protein of claim 1 or the tubule or a tubule or nanotubule, bundle, ball, fiber, filament or sheet of claim 27, a non-derivatized cannulae protein, or a combination thereof.

58: A method for polymerizing the nanotubule, bundle, filament or sheet comprising mixing a plurality the composition of claim 1 in a solution comprising an iron sulfate, a manganese sulfate, a lead sulfate, a lithium sulfate, a manganese chloride or a calcium chloride or an equivalent salt, under conditions wherein the chimeric protein polymerizes into a nanotubule.

59: A fluorescent chimeric polypeptide comprising
(a) at least a first domain comprising a cannulae polypeptide and a second domain comprising a heterologous polypeptide or peptide, wherein the heterologous polypeptide or peptide comprises a fluorescent moiety; or
(b) the fluorescent chimeric polypeptide (a), wherein the fluorescent moiety comprises a green fluorescent protein or equivalent.

60. (canceled)

61: A fluorescent nanotubule, bundle, filament or sheet comprising the fluorescent chimeric polypeptide of claim 59.

62: A bonding or adhesive composition comprising a microarray, filament, sheet, fabric or bundle comprising a plurality of chimeric proteins as set forth in claim 1.

63: A bonding or adhesive composition comprising a microarray, filament, sheet, fabric or bundle comprising a tubule or nanotubule, bundle, ball, fiber, filament or sheet of claim 27.

64: A filter comprising a microarray, filament, sheet, fabric or bundle comprising a tubule or nanotubule, bundle, ball, fiber, filament or sheet of claim 27.

65: A detecting device comprising a microarray, filament, sheet, fabric or bundle comprising a tubule or nanotubule, bundle, ball, fiber, filament or sheet of claim 27.

66: A detoxifying device comprising a microarray, filament, sheet, fabric or bundle comprising a tubule or nanotubule, bundle, ball, fiber, filament or sheet of claim 27.

67: A kit comprising a product of manufacture comprising the composition of claim 1 or a tubule or nanotubule, bundle, ball, fiber, filament or sheet of claim 27, a non-derivatized cannulae protein, or a combination thereof, and instructions for using the product of manufacture.

68: A pharmaceutical composition comprising
(A) (a) the composition of claim 1 or the tubule or nanotubule, bundle, ball, fiber, filament or sheet of claim 27;
(b) the pharmaceutical composition of (a), wherein the at least one additional domain is attached at the amino terminal end, the carboxy terminal end or internal to the cannulae polypeptide; or
(B) (a) a chimeric protein comprising at least a first domain comprising a cannulae polypeptide and at least a second domain comprising a heterologous domain;
(b) the pharmaceutical composition of (a), wherein the heterologous domain is attached at the amino terminal end, the carboxy terminal end or internal to the cannulae polypeptide;
(c) the pharmaceutical composition of (a) or (b), wherein the cannulae polypeptide comprises a protein having at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity to SEQ ID NO:2, SEQ ID NO:4, SEQ ID NO:6, SEQ ID NO:8, SEQ ID NO:10 or SEQ ID NO:12, or a FtsZ protein domain;
(d) the pharmaceutical composition any of (a) to (c), wherein the chimeric polypeptide comprises a recombinant fusion protein and the heterologous domain comprises polypeptide or a peptide; or
(e) the pharmaceutical composition of any of (a) to (d), wherein the heterologous domain of the chimeric polypeptide comprises an epitope, an immunogen, a toleragen, a carbohydrate binding domain, a cell matrix binding domain, a small molecule, a small molecule binding domain, a lipid, a carbohydrate, an enzyme, a cytokine or an antibody.

69-73. (canceled)

74: A vaccine comprising
(a) the composition of claim 1 or the tubule or nanotubule, bundle, ball, fiber, filament or sheet of claim 27, and a pharmaceutically acceptable excipient;
(b) the vaccine of (a), wherein the at least one additional domain of the composition comprises an epitope, an immunogen, a toleragen, an immunomodulatory agent, an immune suppression agent, an adjuvant, an antibody, a cell binding agent, a carbohydrate or a combination thereof; or
(c) the vaccine of (a) or (b), wherein the chimeric polypeptide is assembled or self-assembles into a tubule or nanotubule, bundle, ball, fiber, filament or sheet.

75-76. (canceled)

77: A method for modulating the immune system of an individual comprising
(a) administering a pharmaceutically effective amount of the composition of claim 1, the pharmaceutical composition of claim 68 or the vaccine of claim 74, to an individual in need thereof;
(b) the method of (a), wherein a humoral or a cell-based immune response is elicited in the individual; or
(c) the method of (a) or (b), wherein the individual is a human.

78-79. (canceled)

80: A carbohydrate-based therapeutic pharmaceutical composition comprising
(a) the composition of claim 1, or the tubule or nanotubule, bundle, ball, fiber, filament, thread, or sheet of claim 27, wherein the composition, tubule or nanotubule, bundle, ball, fiber, filament, thread, or sheet comprises at least one carbohydrate; or
(b) the carbohydrate-based therapeutic pharmaceutical composition of (a), wherein the composition, tubule or nanotubule, bundle, ball, fiber, filament, thread, or sheet comprises a polypeptide or peptide having a carbohydrate-binding motif;
(c) the carbohydrate-based therapeutic pharmaceutical composition of (a) or (b), wherein the carbohydrate-binding motif is an N-linked carbohydrate-binding motif or an O-linked carbohydrate-binding motif; or
(d) the carbohydrate-based therapeutic pharmaceutical composition of any of (a) to (c), wherein the carbohydrate is added chemically, by cellular biosynthetic mechanisms, by in vitro enzymatic reactions, or a combination thereof.

81-83. (canceled)

84: A method for ameliorating a disease or condition comprising
(a) administering a pharmaceutically effective amount of the carbohydrate-based therapeutic pharmaceutical composition of claim 80 to an individual; or
(b) the method of (a), wherein ameliorating the disease or condition comprises inhibition of carbohydrate-lectin interactions; immunization with carbohydrate antigens; inhibition of enzymes that synthesize disease-associated carbohydrates; inhibition of carbohydrate-processing enzymes; targeting of drugs to specific disease cells via carbohydrate-lectin interactions; administering carbohydrate based anti-thrombotic agents.

85. (canceled)

86: A cell matrix binding composition comprising
(a) the composition of claim 1, or the tubule or nanotubule, bundle, ball, fiber, filament, thread, or sheet of claim 27, wherein the composition, tubule or nanotubule, bundle, ball, fiber, filament, thread, or sheet comprises at least one a cell matrix binding motif;
(b) the cell matrix binding composition of (a), wherein the cell matrix binding motif comprises an RGD-binding motif or an RGD motif;
(c) the cell matrix binding composition of (a) or (b), comprising a medical device; or
(d) the cell matrix binding composition of (c), wherein the medical device comprises a dental or orthopedic prostheses, a dental device or implant, an orthopedic device, a pin, a screw, a fixture, a plate, a stent, a stent sheath, a shunt, a catheter, a valve, a cannulae, a tissue scaffold, a wound care device, a dressing or a lens.

87-89. (canceled)

90: A tissue scaffold or implant material comprising
(a) the composition of claim 1, or the tubule or nanotubule, bundle, ball, fiber, filament, thread, or sheet of claim 27;
(b) the tissue scaffold or implant material of (a), wherein the tissue scaffold comprises a polymer scaffold and neural stem cells for repairing a spinal cord injury;
(c) the tissue scaffold or implant material of (a) or (b), wherein the tissue scaffold comprises a vascular graft comprising graft material from smooth muscle, endothelial muscle and/or stem cells.

91-92. (canceled)

93: A cell or tissue transplant device or a cell or tissue implant device comprising
(a) the composition of claim 1, or the tubule or nanotubule, bundle, ball, fiber, filament, thread, or sheet of claim 27; or
(b) the cell or tissue transplant device or a cell or tissue implant device of (a), wherein the cells or tissues comprise nerve cells or tissues, skin cells or tissues, epidermal cells, dermal cells, liver cells or tissue; kidney cells or tissue; pancreatic cells or tissues; tubular structural cells, vascular elements, arteries, arterioles, veins, ureter cells or structure, bladder cells, urethral or structure, ductal tissue, bone cells or tissue, cartilage cells and/or muscle cells or tissue.

94-95. (canceled)

96: A bottle-brush polymeric protein structure comprising the composition of claim 1 and a FtsZ domain.

97: A chromatography resin comprising
(a) the composition of claim 1; or
(b) the composition of claim 1 and a FtsZ domain.

98. (canceled)
